(19) United States
(12) Patent Application Publication
(10) Pub. No.: US 2026/0327270 A1
(43) Pub. Date: Oct. 22, 2026

(54) AAV CAPSID VARIANTS FOR GENE THERAPY

(71) Applicant: University of Florida Research Foundation, Incorporated, Gainesville, FL (US)

(72) Inventor: Hugh Lee Sweeney, Gainesville, FL (US)

(73) Assignee: University of Florida Research Foundation, Incorporated, Gainesville, FL (US)

(21) Appl. No.: 19/631,086

(22) Filed: Mar. 27, 2026

Related U.S. Application Data
(63) Continuation of application No. 17/635,151, filed on Aug. 14, 2020, now Pat. No. 12,612,432.
(60) Provisional application No. 62/886,915, filed on Aug. 14, 2019.

(51) Int. Cl. C07K 14/005; C12N 15/86

(52) U.S. Cl. CPC C07K 14/005; C12N 15/86; C12N 2750/14122; C12N 2750/14143; C12N 2750/14145; C12N 2750/14171

(57) ABSTRACT

Adeno associated viral (AAV) particles are emerging as a useful vehicle for gene delivery to various organs and tissues. Provided here are variant AAV capsid proteins and variant capsid protein containing particles. Compositions of these variant AAV particles can be used to alter tissue tropism and transduction efficiency.

### RELATED APPLICATIONS

This application is a continuation of U.S. application Ser. No. 17/635,151, filed Feb. 14, 2022, entitled “AAV CAPSID VARIANTS FOR GENE THERAPY”, which is a national stage filing under 35 U.S.C. § 371 of International Patent Application Serial No. PCT/US2020/046543, filed Aug. 14, 2020, entitled “AAV CAPSID VARIANTS FOR GENE THERAPY”, which claims the benefit under 35 U.S.C. § 119(e) of the filing date of U.S. Provisional Application Ser. No. 62/886,915, filed Aug. 14, 2019, entitled “AAV CAPSID VARIANTS FOR GENE THERAPY”, the entire contents of each of which are incorporated herein by reference.

### REFERENCE TO AN ELECTRONIC SEQUENCE LISTING

The contents of the electronic sequence listing (U119770161US02-SEQ-COB.xml; Size: 81,815 bytes; and Date of Creation: Mar. 6, 2026) are herein incorporated by reference in their entirety.

### BACKGROUND

Recombinant adeno-associated viruses (rAAV) have moved into the clinic as gene transfer vectors for a number of different diseases. While in some cases the vectors are delivered directly into the tissue of interest, in a growing number of diseases the delivery is systemic. Thus, the development of new AAV variants of with differing tissue tropisms, as well as higher efficiencies of transduction, is highly desirable.

### SUMMARY

Disclosed herein are variant recombinant adeno-associated virus (rAAV) capsid proteins, nucleic acids encoding such proteins, rAAV particles comprising such proteins (e.g., encapsidating a rAAV genome encoding a gene of interest). In some embodiments, compositions described in this application are useful to produce rAAV particles, and/or to deliver one or more genes of interest to a target tissue (e.g., to a muscle tissue, for example to cardiac or skeletal muscle).

When used for the treatment of muscle diseases, large amounts of virus are needed to transduce the skeletal and/or cardiac muscles of the body. Unfortunately, large amounts of virus can also trigger a number of different types of undesirable immune responses that can be life threatening to patients. Accordingly, there is a need to optimize the tropism of the virus for the tissue of interest, while decreasing uptake of virus in non-targeted tissues.

In some embodiments, disclosed herein are variant recombinant adeno-associated virus (rAAV) serotype 8 (AAV8) capsid proteins having one or more amino acid substitutions that enhance tissue tropism (e.g., muscle tropism). In some embodiments, rAAV capsid proteins comprise one or more additional amino acid substitutions that reduce brain and/or liver tropism.

In some embodiments, the rAAV8 capsid proteins include one or more of any one or more of the changes as shown in FIG. 1. In some embodiments, the rAAV8 capsid protein comprises an amino acid sequence as set forth in SEQ ID NO: 11.

In some embodiments, the rAAV8 capsid proteins further comprise any one or more of the following changes: N500I; N263S; G264A; T265S; S266T; deletion of G268; T270S; and T274H. In some embodiments, the rAAV8 capsid protein comprises an amino acid sequence as set forth in SEQ ID NO: 15. In some embodiments, the rAAV8 capsid protein comprises an amino acid sequence as set forth in SEQ ID NO: 21. In some embodiments, the rAAV8 capsid protein comprises an amino acid sequence as set forth in SEQ ID NO: 27.

In some embodiments, the rAAV8 capsid proteins include one or more of the following substitutions: A24D, D41N, Q84K, R92K, T158A, K163S, R169K, L189I, A195G, V199L, P201S, S225A, G264S, A269S, R313K, S315N, E350D, Q362E, Q413E, T415S, T417Q, Y447F, T453S, N459G, T462Q, G464L, N471S, T472N, A474S, N475A, T494V, A507G, G508A, N517D, A520V, T528S, D531E, D532G, N540S, N549G, A551G, A555V, D559K, E578Q, I581Q, T591A, Q594I, 1595V, N665S, S667A, N670A, S712N, V722T, and Y733F. In some embodiments, the rAAV8 capsid protein comprises an amino acid sequence as set forth in SEQ ID NO: 12.

In some embodiments, the rAAV8 capsid proteins further comprise any one or more of the following changes: N500I; N263S; S264A; T265S; S266T; deletion of G268; S269A; T270S; and T274H. In some embodiments, the rAAV8 capsid protein comprises an amino acid sequence as set forth in SEQ ID NO: 17. In some embodiments, the rAAV8 capsid protein comprises an amino acid sequence as set forth in SEQ ID NO: 23. In some embodiments, the rAAV8 capsid protein comprises an amino acid sequence as set forth in SEQ ID NO: 29.

In some embodiments, the rAAV8 capsid protein includes one or more of the following substitutions: K31Q, D41N, G42A, Q84K, R92K, Q105K, T158A, K163S, S180T, P201S, S225A, G264S, A269S, R313K, S315N, E350D, Q362E, Q413E, T415S, T417Q, Y447F, T453S, N459G, T462Q, G464L, N471S, T472N, A474S, N475A, T494V, A507G, G508A, N517D, A520V, T528S, D531E, D532G, N540S, N549G, A551G, A555V, D559K, 1581Q, Q588S, Q589A, Q594I, 1595V, N665S, S667A, N670A, S712N, V722T, and Y733F. In some embodiments, the rAAV8 capsid protein comprises an amino acid sequence as set forth in SEQ ID NO: 13.

In some embodiments, the rAAV8 capsid proteins further comprise any one or more of the following changes: N500I; N263S; S264A; T265S; S266T; deletion of G268; S269A; T270S; and T274H. In some embodiments, the rAAV8 capsid protein comprises an amino acid sequence as set forth in SEQ ID NO: 19. In some embodiments, the rAAV8 capsid protein comprises an amino acid sequence as set forth in SEQ ID NO: 25. In some embodiments, the rAAV8 capsid protein comprises an amino acid sequence as set forth in SEQ ID NO: 31.

Further disclosed are variant rAAV capsid proteins of a serotype other than serotype 8 (e.g., AAV2, AAV3, AAV4, AAV5, AAV6, AAV7, AAV9, AAV10, AAVrh10, AAVrh74, AAVanc80L65, etc.) comprising any one or more of the disclosed amino acid changes. In some embodiments, the amino acid substitution(s) are at position(s) in the capsid protein of a serotype other than serotype 8 corresponding to the position(s) of the substitution(s) in AAV8. Accordingly, in some embodiments, one or more (e.g., 2, 3, 4, 5, 6, 7, 8, 9, 10, or more) of the amino acid substitutions illustrated in FIG. 1, FIG. 4, and or SEQ ID NOs: 2, 4, 6, 11-13, 15, 17, 19, 21, 23, 25, 27, 29, and/or 31 can be included in an rAAV8 based capsid protein, or in an rAAV capsid protein that is based on a different serotype (e.g., in any of the capsid proteins set forth in SEQ ID NOs: 7-10), and/or related rAAV particles (e.g., comprising a nucleic acid encoding a gene of interest). In some embodiments, a variant capsid protein can be a variant VP1, VP2, or VP3 capsid protein. In some embodiments, a variant capsid protein comprises a subset of the amino acid substitutions illustrated in FIG. 1 and FIG. 4, for example a subset that has 1, 2, 3, 4, 5, 6, 7, 8, 9, or 10 fewer amino acid substitutions than those illustrated in FIG. 1 and FIG. 4.

Also disclosed are variant recombinant AAV (e.g., rAAV8) particles comprising the recombinant AAV (e.g., rAAV8) capsid proteins disclosed. In some embodiments, the AAV (e.g., rAAV8) particles include a nucleic acid comprising a gene of interest. In some embodiments, the nucleic acid is single stranded. In some embodiments, the nucleic acid is double stranded.

Also disclosed are compositions comprising a plurality of the variant recombinant AAV (e.g., rAAV8) particles disclosed herein. These compositions may include a pharmaceutically acceptable carrier.

Also provided are methods of transducing a cell with a gene of interest, comprising providing to the cell a composition disclosed herein, wherein the AAV (e.g., rAAV8) particles in the composition comprise the gene of interest. Further provided are methods of transducing a cell with a gene of interest, comprising providing to the cell a composition comprising a plurality of recombinant AAV (e.g., rAAV8) particles comprising a variant recombinant AAV (e.g., rAAV8) capsid protein disclosed herein, and wherein the AAV (e.g., rAAV8) particles in the composition comprise the gene of interest. In some embodiments, the gene of interest encodes a therapeutic protein. In some embodiments, the therapeutic protein is an antibody or antibody fragment, a peptibody, a growth factor, a hormone, a membrane protein, a cytokine, a chemokine, an activating or inhibitory peptide acting on cell surface receptors or ion channels, a cell-permeant peptide targeting intracellular processes, an enzyme, a nuclease or other protein used for gene editing. In some embodiments, the subject is a mammal. In some embodiments, the subject is a human.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1E shows an alignment of AAV variants SL1.2, SL2, and SL3 with other commonly used AAV vectors AAV8, AAV9, AAVrh10, AAVrh74, and AAVanc80L65. Red indicates identical amino acids across all capsids compared; orange indicates that one of the sequences compared substitutes a different amino acid; green indicates that two of the sequences compared substitute a different amino acid; blue indicates that three or more of the sequences compared substitute a different amino acid.

FIG. 2 depicts a “phylogenetic tree” of AAV variants SL1.2, SL2, SL3, AAV8, AAV9, AAVrh10, AAVrh74, and AAVanc80L65 based on the sequence of their capsid protein.

FIG. 3 shows the percent sequence homology/divergence of AAV variants SL1.2, SL2, SL3, AAV8, AAV9, AAVrh10, AAVrh74, and AAVanc80L65.

FIGS. 4A-4J show an alignment of AAV variants SL1.2, SL1.2L, SL1.2B, SL1.2 LB, SL2, SL2L, SL2B, SL2LB, SL3, SL3L, SL3B, and SL3LB with other commonly used AAV vectors AAV8, AAV9, AAVrh10, AAVrh74, and AAVanc80L65. The “L” suffix refers to the single amino acid change that has been reported to result in less efficient liver targeting. The “B” suffix refers to an eight amino acid substitution/deletion that has been reported to impair crossing of the blood-brain barrier. The “LB” suffix refers to the single amino acid change that has been reported to result in less efficient liver targeting (L) combined with an eight amino acid substitution/deletion that has been reported to impair crossing of the blood-brain barrier. Red indicates identical amino acids across all capsids compared; orange indicates that one of the sequences compared substitutes a different amino acid; green indicates that two of the sequences compared substitute a different amino acid; blue indicates that three or more of the sequences compared substitute a different amino acid.

FIG. 5 depicts a “phylogenetic tree” of AAV variants SL1.2, SL1.2L, SL1.2B, SL1.2 LB, SL2, SL2L, SL2B, SL2LB, SL3, SL3L, SL3B, SL3LB, AAV8, AAV9, AAVrh10, AAVrh74, and AAVanc80L65 based on the sequence of their capsid protein.

FIG. 6 shows the percent sequence homology/divergence of AAV variants SL1.2, SL1.2L, SL1.2B, SL1.2 LB, SL2, SL2L, SL2B, SL2LB, SL3, SL3L, SL3B, SL3LB, AAV8, AAV9, AAVrh10, AAVrh74, and AAVanc80L65.

FIG. 7 shows the Western blot quantification of the blot shown in FIG. 8. The delivery of the three variants (SL1.2, SL2, and SL3) is compared to the uptake of the natural serotypes, AAV9 and AAVrh10, and the engineered variant, AAVanc80L65. The experiment was conducted using a known sub-saturating dose (1×10¹³ vg/kg) administered by tail vein systemic delivery into 6-month-old DBA/2J male mice. Green fluorescent protein (GFP) was used as the reporter protein under control of a beta-actin promoter for expression in all tissues. The variant SL2 has the improved uptake in cardiac tissue relative to the control serotypes, while SL3 has uptake more similar to AAV9. SL3 has highest uptake in skeletal muscle and brain of any of tested AAV serotype.

FIG. 8 shows the Western Blot data corresponding to the quantification shown in FIG. 7.

FIG. 9 shows the superiority of SL3 in facilitating uptake in both skeletal and cardiac muscles. A Western blot and quantification of heart and diaphragm are shown. The data was obtained by injecting 1×10¹³ vg/kg of AAV9, SL1.2, SL2, or SL3 by tail vein (systemic delivery) into 6-month-old DBA/2J male mice. Green fluorescent protein (GFP) was used as the reporter protein under control of a beta-actin promoter for expression in all tissues.

FIG. 10 shows the superiority of SL3 in facilitating uptake in both skeletal and cardiac muscles. A Western blot and quantification of quadriceps are shown. The data was obtained by injecting 1×10¹³ vg/kg of AAV9, SL1.2, SL2, or SL3 by tail vein (systemic delivery) into 6-month-old DBA/2J male mice. Green fluorescent protein (GFP) was used as the reporter protein under control of a beta-actin promoter for expression in all tissues.

FIG. 11 demonstrates the distribution of Green Fluorescent Protein (GFP; degree of brightness) within skeletal muscles and the heart following systemic injection of AAV9, SL1.2, SL2, or SL3 at 5×10¹³ vg/kg or 1× 10¹⁴ vg/kg. The pattern of expression supports the conclusions of the Western blot data, and indicate that SL3 is the superior vector for transduction of the heart and skeletal muscles.

### DETAILED DESCRIPTION

AAV-derived vectors are promising tools for human gene therapy applications because of reduced pathogenicity compared to other vectors, episomal localization, and stable transgene expression. AAV particles show huge promise for the delivery of therapeutic genes. The organ or tissue tropism of AAV particles depends highly, if not entirely, on the make-up of the particle surface, or the capsid. Disclosed herein are AAV variants that alter the tropisms and/or efficiencies of AAV transduction as compared to AAV8. These vectors can be used in the clinic for either veterinary or human use. The tissue tropism may differ depending on the mammalian species. The basis of the differences in tissue tropism and efficiencies is based primarily on sequence variations in surface loops.

AAV serotype 8 (AAV8) has a tropism for and may be used to deliver genes to skeletal muscle, retinal pigment epithelium, photoreceptors, cardiac tissue, and hepatocytes. As is typical for AAVs, the AAV8 capsid is made up of three proteins, VP1, VP2 and VP3, the product of three different but overlapping transcripts of the single AAV cap gene. Provided herein are compositions and methods for variant recombinant AAV8-like capsid proteins and particles that alter the tropisms and/or efficiencies of AAV transduction as compared to AAV8. This disclosure is based, at least in part, on the identification of recombinant AAV (e.g., rAAV8) variant proteins and particles that alter tissue tropism and alter transduction efficiency compared to wild-type rAAV (e.g., rAAV8) protein and particles.

As used herein, the term “variant” refers to a nucleic acid or protein having characteristics that deviate from what occurs in nature, e.g., a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type nucleic acid or protein. For instance, a transgene variant is a nucleic acid comprising one or more substitutions in the nucleotides of a transgene, as compared to the wild type sequence thereof. These substitutions may be silent, i.e. they do not modify the amino acid sequence of any encoded protein (or otherwise result in a variant amino acid sequence). Alternatively, these substitutions may result in modifications to the amino acid sequence of an encoded protein, resulting in an encoded protein having one or more amino acid substitutions (e.g., having 1, 2, 3, 4, 5, 6, 7, 8, 9, 10, 10-15, or 15-20 amino acid substitutions) relative to the wild type protein sequence. These substitutions include chemical modifications as well as truncations. This term further embraces functional fragments of a wild type nucleic acid or amino acid sequence. These modifications of the reference sequence may occur at the 5′ or 3′ ends of the reference sequence or anywhere between those positions, interspersed either individually among nucleotides or peptides in the reference sequence or in one or more contiguous groups within the reference sequence.

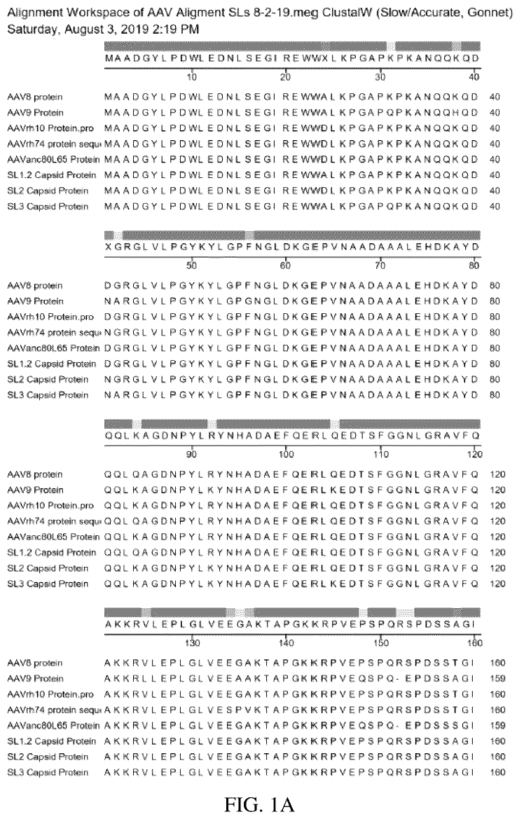

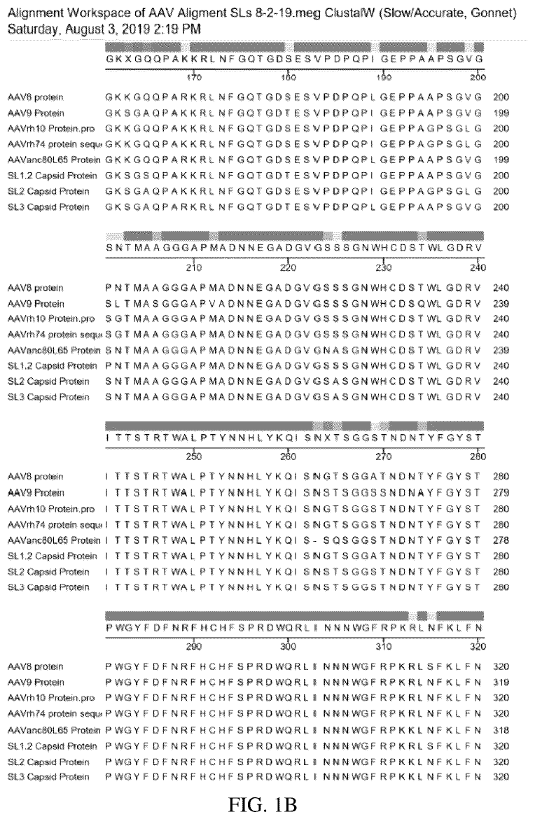

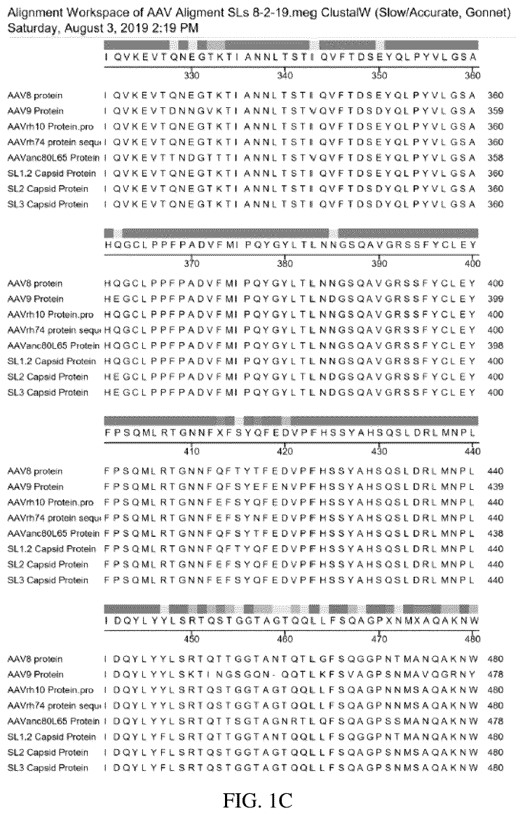

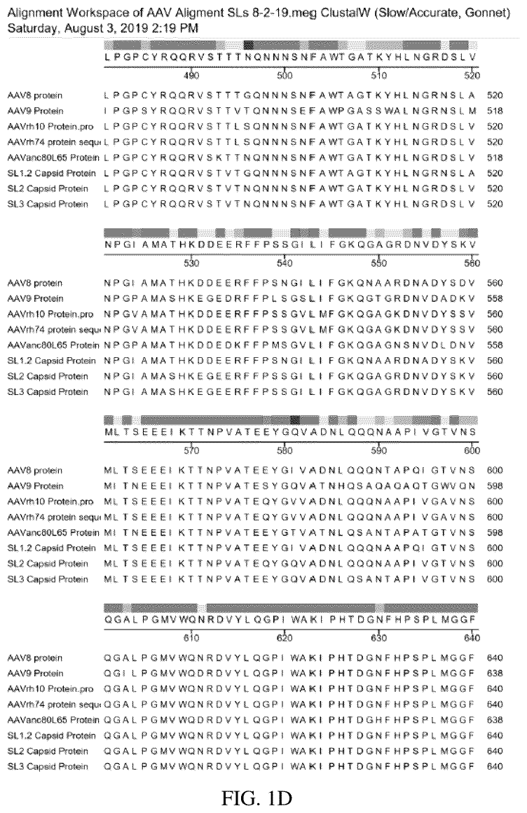

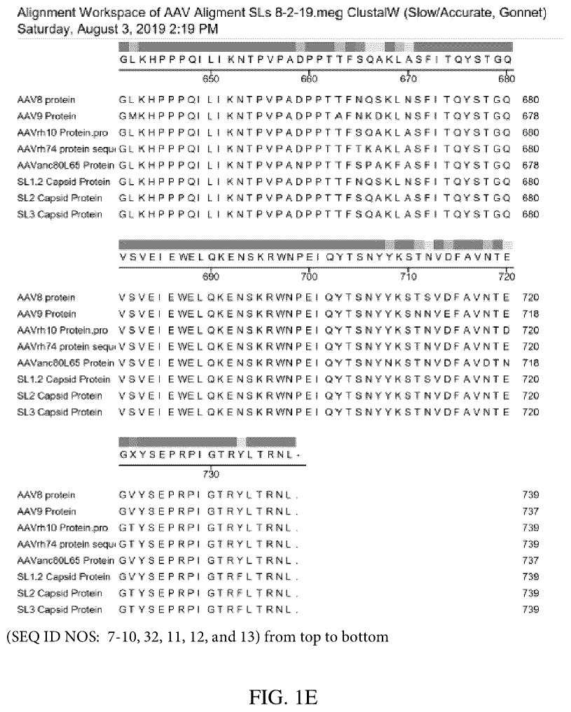

In some embodiments, the variant recombinant adeno-associated virus (rAAV) capsid proteins disclosed herein have a Y to F substitution at position 447 and a T to V substitution at position 494 made in a background comprising serotype 8 (AAV8) VP1 or VP2 or VP3. In some embodiments, the variant recombinant AAV (e.g., rAAV8) capsid protein comprises the amino acid sequence set forth in SEQ ID NO: 11. In some embodiments, the variant recombinant AAV (e.g., rAAV8) capsid protein has an amino acid sequence identified as SL1.2 Capsid Protein in FIGS. 1A-1E. In some embodiments, the variant recombinant AAV (e.g., rAAV8) capsid protein includes one or more of the following substitutions: K163S, R169K, S180T, L189I, T417Q, Y447F, T462Q, G464L, T494V, D559K, T591A, and Y733F. In one embodiment, the variant recombinant AAV (e.g., rAAV8) capsid protein includes substitutions K163S, R169K, S180T, L189I, T417Q, Y447F, T462Q, G464L, T494V, D559K, T591A, and Y733F.

In some embodiments, the variant recombinant AAV (e.g., rAAV8) capsid protein includes one or more of the following substitutions: A24D, D41N, Q84K, R92K, T158A, K163S, R169K, L189I, A195G, V199L, P201S, S225A, G264S, A269S, R313K, S315N, E350D, Q362E, Q413E, T415S, T417Q, Y447F, T453S, N459G, T462Q, G464L, N471S, T472N, A474S, N475A, T494V, A507G, G508A, N517D, A520V, T528S, D531E, D532G, N540S, N549G, A551G, A555V, D559K, E578Q, 1581Q, T591A, Q594I, 1595V, N665S, S667A, N670A, S712N, V722T, and Y733F. In one embodiment, the variant recombinant AAV (e.g., rAAV8) capsid protein includes substitutions A24D, D41N, Q84K, R92K, T158A, K163S, R169K, L189I, A195G, V199L, P201S, S225A, G264S, A269S, R313K, S315N, E350D, Q362E, Q413E, T415S, T417Q, Y447F, T453S, N459G, T462Q, G464L, N471S, T472N, A474S, N475A, T494V, A507G, G508A, N517D, A520V, T528S, D531E, D532G, N540S, N549G, A551G, A555V, D559K, E578Q, 1581Q, T591A, Q594I, 1595V, N665S, S667A, N670A, S712N, V722T, and Y733F. In some embodiments, the variant recombinant AAV (e.g., rAAV8) comprises the amino acid sequence set forth in SEQ ID NO: 12. In some embodiments, the variant recombinant AAV (e.g., rAAV8) capsid protein has an amino acid sequence identified as SL2 Capsid Protein in FIGS. 1A-1E.

In some embodiments, the variant rAAV capsid protein has a Y to F substitution at position 447 and a T to V substitution at position 494 made in a background comprising serotype 8 (AAV8) VP1, VP2 or VP3, and includes any one or more of the following substitutions: K31Q, D41N, G42A, Q84K, R92K, Q105K, T158A, K163S, S180T, P201S, S225A, G264S, A269S, R313K, S315N, E350D, Q362E, Q413E, T415S, T417Q, Y447F, T453S, N459G, T462Q, G464L, N471S, T472N, A474S, N475A, T494V, A507G, G508A, N517D, A520V, T528S, D531E, D532G, N540S, N549G, A551G, A555V, D559K, 1581Q, Q588S, Q589A, Q594I, 1595V, N665S, S667A, N670A, S712N, V722T, and Y733F. In one embodiment, the variant recombinant AAV (e.g., rAAV8) capsid protein includes substitutions K31Q, D41N, G42A, Q84K, R92K, Q105K, T158A, K163S, S180T, P201S, S225A, G264S, A269S, R313K, S315N, E350D, Q362E, Q413E, T415S, T417Q, Y447F, T453S, N459G, T462Q, G464L, N471S, T472N, A474S, N475A, T494V, A507G, G508A, N517D, A520V, T528S, D531E, D532G, N540S, N549G, A551G, A555V, D559K, 1581Q, Q588S, Q589A, Q594I, 1595V, N665S, S667A, N670A, S712N, V722T, and Y733. In some embodiments, the variant recombinant AAV (e.g., rAAV8) comprises the amino acid sequence set forth in SEQ ID NO: 13. In some embodiments, the variant recombinant AAV (e.g., rAAV8) capsid protein has an amino acid sequence identified as SL3 Capsid Protein in FIGS. 1A-1E.

### AAV8 Structure and Capsid Proteins

The wild-type AAV genome is built of single-stranded deoxyribonucleic acid (ssDNA), which is either positive- or negative-sensed. At each end of the DNA strand is an inverted terminal repeat (ITR). Between the ITRs are two open reading frames (ORFs): rep and cap. The rep ORF is composed of four overlapping genes encoding Rep proteins required for the AAV life cycle. The cap ORF contains overlapping nucleotide sequences of capsid proteins: VP1, VP2 and VP3, which interact together to form a capsid of an icosahedral symmetry.

Wild-type AAV8 amino acid sequence:
(SEQ ID NO: 7)
MAADGYLPDWLEDNLSEGIREWWALKPGAPKPKAN
QQKQDDGRGLVLPGYKYLGPFNGLDKGEPVNAADA
AALEHDKAYDQQLQAGDNPYLRYNHADAEFQERLQ
EDTSFGGNLGRAVFQAKKRVLEPLGLVEEGAKTAP
GKKRPVEPSPQRSPDSSTGIGKKGQQPARKRLNFG
QTGDSESVPDPQPLGEPPAAPSGVGPNTMAAGGGA
PMADNNEGADGVGSSSGNWHCDSTWLGDRVITTST
RTWALPTYNNHLYKQISNGTSGGATNDNTYFGYST
PWGYFDFNRFHCHFSPRDWQRLINNNWGFRPKRLS
FKLFNIQVKEVTQNEGTKTIANNLTSTIQVFTDSE
YQLPYVLGSAHQGCLPPFPADVFMIPQYGYLTLNN
GSQAVGRSSFYCLEYFPSQMLRTGNNFQFTYTFED
VPFHSSYAHSQSLDRLMNPLIDQYLYYLSRTQTTG
GTANTQTLGFSQGGPNTMANQAKNWLPGPCYRQQR
VSTTTGQNNNSNFAWTAGTKYHLNGRNSLANPGIA
MATHKDDEERFFPSNGILIFGKQNAARDNADYSDV
MLTSEEEIKTTNPVATEEYGIVADNLQQQNTAPQI
GTVNSQGALPGMVWQNRDVYLQGPIWAKIPHTDGN
FHPSPLMGGFGLKHPPPQILIKNTPVPADPPTTFN
QSKLNSFITQYSTGQVSVEIEWELQKENSKRWNPE
IQYTSNYYKSTSVDFAVNTEGVYSEPRPIGTRYLT
RNL
Wild-type AAV9 amino acid sequence:
(SEQ ID NO: 8)
MAADGYLPDWLEDNLSEGIREWWALKPGAPQPKAN
QQHQDNARGLVLPGYKYLGPGNGLDKGEPVNAADA
AALEHDKAYDQQLKAGDNPYLKYNHADAEFQERLK
EDTSFGGNLGRAVFQAKKRLLEPLGLVEEAAKTAP
GKKRPVEQSPQEPDSSAGIGKSGAQPAKKRLNFGQ
TGDTESVPDPQPIGEPPAAPSGVGSLTMASGGGAP
VADNNEGADGVGSSSGNWHCDSQWLGDRVITTSTR
TWALPTYNNHLYKQISNSTSGGSSNDNAYFGYSTP
WGYFDFNRFHCHFSPRDWQRLINNNWGFRPKRLNF
KLFNIQVKEVTDNNGVKTIANNLTSTVQVFTDSDY
QLPYVLGSAHEGCLPPFPADVFMIPQYGYLTLNDG
SQAVGRSSFYCLEYFPSQMLRTGNNFQFSYEFENV
PFHSSYAHSQSLDRLMNPLIDQYLYYLSKTINGSG
QNQQTLKFSVAGPSNMAVQGRNYIPGPSYRQQRVS
TTVTQNNNSEFAWPGASSWALNGRNSLMNPGPAMA
SHKEGEDRFFPLSGSLIFGKQGTGRDNVDADKVMI
TNEEEIKTTNPVATESYGQVATNHQSAQAQAQTGW
VQNQGILPGMVWQDRDVYLQGPIWAKIPHTDGNFH
PSPLMGGFGMKHPPPQILIKNTPVPADPPTAFNKD
KLNSFITQYSTGQVSVEIEWELQKENSKRWNPEIQ
YTSNYYKSNNVEFAVNTEGVYSEPRPIGTRYLTRN
L
AAVrh10 amino acid sequence:
(SEQ ID NO: 9)
MAADGYLPDWLEDNLSEGIREWWDLKPGAPKPKAN
QQKQDDGRGLVLPGYKYLGPFNGLDKGEPVNAADA
AALEHDKAYDQQLKAGDNPYLRYNHADAEFQERLQ
EDTSFGGNLGRAVFQAKKRVLEPLGLVEEGAKTAP
GKKRPVEPSPQRSPDSSTGIGKKGQQPAKKRLNFG
QTGDSESVPDPQPIGEPPAGPSGLGSGTMAAGGGA
PMADNNEGADGVGSSSGNWHCDSTWLGDRVITTST
RTWALPTYNNHLYKQISNGTSGGSTNDNTYFGYST
PWGYFDFNRFHCHFSPRDWQRLINNNWGFRPKRLN
FKLFNIQVKEVTQNEGTKTIANNLTSTIQVFTDSE
YQLPYVLGSAHQGCLPPFPADVFMIPQYGYLTLNN
GSQAVGRSSFYCLEYFPSQMLRTGNNFEFSYQFED
VPFHSSYAHSQSLDRLMNPLIDQYLYYLSRTQSTG
GTAGTQQLLFSQAGPNNMSAQAKNWLPGPCYRQQR
VSTTLSQNNNSNFAWTGATKYHLNGRDSLVNPGVA
MATHKDDEERFFPSSGVLMFGKQGAGKDNVDYSSV
MLTSEEEIKTTNPVATEQYGVVADNLQQQNAAPIV
GAVNSQGALPGMVWQNRDVYLQGPIWAKIPHTDGN
FHPSPLMGGFGLKHPPPQILIKNTPVPADPPTTFS
QAKLASFITQYSTGQVSVEIEWELQKENSKRWNPE
IQYTSNYYKSTNVDFAVNTDGTYSEPRPIGTRYLT
RNL
AAVrh74 amino acid sequence:
(SEQ ID NO: 10)
MAADGYLPDWLEDNLSEGIREWWDLKPGAPKPKAN
QQKQDNGRGLVLPGYKYLGPFNGLDKGEPVNAADA
AALEHDKAYDQQLQAGDNPYLRYNHADAEFQERLQ
EDTSFGGNLGRAVFQAKKRVLEPLGLVESPVKTAP
GKKRPVEPSPQRSPDSSTGIGKKGQQPAKKRLNFG
QTGDSESVPDPQPIGEPPAGPSGLGSGTMAAGGGA
PMADNNEGADGVGSSSGNWHCDSTWLGDRVITTST
RTWALPTYNNHLYKQISNGTSGGSTNDNTYFGYST
PWGYFDFNRFHCHFSPRDWQRLINNNWGFRPKRLN
FKLFNIQVKEVTQNEGTKTIANNLTSTIQVFTDSE
YQLPYVLGSAHQGCLPPFPADVFMIPQYGYLTLNN
GSQAVGRSSFYCLEYFPSQMLRTGNNFEFSYNFED
VPFHSSYAHSQSLDRLMNPLIDQYLYYLSRTQSTG
GTAGTQQLLFSQAGPNNMSAQAKNWLPGPCYRQQR
VSTTLSQNNNSNFAWTGATKYHLNGRDSLVNPGVA
MATHKDDEERFFPSSGVLMFGKQGAGKDNVDYSSV
MLTSEEEIKTTNPVATEQYGVVADNLQQQNAAPIV
GAVNSQGALPGMVWQNRDVYLQGPIWAKIPHTDGN
FHPSPLMGGFGLKHPPPQILIKNTPVPADPPTTFT
KAKLASFITQYSTGQVSVEIEWELQKENSKRWNPE
IQYTSNYYKSTNVDFAVNTEGTYSEPRPIGTRYLT
RNL
AAVanc80L65 amino acid sequence:
(SEQ ID NO: 32)
MAADGYLPDWLEDNLSEGIREWWDLKPGAPKPKAN
QQKQDDGRGLVLPGYKYLGPFNGLDKGEPVNAADA
AALEHDKAYDQQLKAGDNPYLRYNHADAEFQERLQ
EDTSFGGNLGRAVFQAKKRVLEPLGLVEEGAKTAP
GKKRPVEQSPQEPDSSSGIGKKGQQPARKRLNFGQ
TGDSESVPDPQPLGEPPAAPSGVGSNTMAAGGGAP
MADNNEGADGVGNASGNWHCDSTWLGDRVITTSTR
TWALPTYNNHLYKQISSQSGGSTNDNTYFGYSTPW
GYFDFNRFHCHFSPRDWQRLINNNWGFRPKKLNFK
LFNIQVKEVTTNDGTTTIANNLTSTVQVFTDSEYQ
LPYVLGSAHQGCLPPFPADVFMIPQYGYLTLNNGS
QAVGRSSFYCLEYFPSQMLRTGNNFQFSYTFEDVP
FHSSYAHSQSLDRLMNPLIDQYLYYLSRTQTTSGT
AGNRTLQFSQAGPSSMANQAKNWLPGPCYRQQRVS
KTTNQNNNSNFAWTGATKYHLNGRDSLVNPGPAMA
THKDDEDKFFPMSGVLIFGKQGAGNSNVDLDNVMI
TNEEEIKTTNPVATEEYGTVATNLQSANTAPATGT
VNSQGALPGMVWQDRDVYLQGPIWAKIPHTDGHFH
PSPLMGGFGLKHPPPQILIKNTPVPANPPTTFSPA
KFASFITQYSTGQVSVEIEWELQKENSKRWNPEIQ
YTSNYNKSTNVDFAVDTNGVYSEPRPIGTRYLTRN
L

### Variant Recombinant AAV8 Capsid Proteins

Provided herein are nucleic acids that encode any one of the variant rAAV capsid proteins disclosed herein. In some embodiments, a plasmid comprises a nucleic acid encoding a variant rAAV capsid protein.

Some non-limiting examples of nucleic acids encoding variant AAV capsid proteins are provided in Table 1. These variants are referred to as SL1.2 (encoded by SEQ ID NO: 1), SL1.2L (encoded by SEQ ID NO: 14), SL1.2B (encoded by SEQ ID NO: 20), SL1.2 LB (encoded by SEQ ID NO: 26), SL2 (encoded by SEQ ID NO: 3), SL2L (encoded by SEQ ID NO: 16), SL2B (encoded by SEQ ID NO: 22), SL2LB (encoded by SEQ ID NO: 28), SL3 (encoded by SEQ ID NO: 5), SL3L (encoded by SEQ ID NO: 18), SL3B (encoded by SEQ ID NO: 24), and SL2LB (encoded by SEQ ID NO: 28).

The tissue tropism and transduction efficiency of AAV particles is determined by the nature of amino acid residues exposed at the surface of the capsid (Wu et al., J Virol. 2006, 80 (22): 11393-7). Therefore, manipulating the amino acids of the capsid proteins provides an opportunity to fine tune the tissue tropism of the particle and also improve transduction efficiency. However, certain manipulations, e.g., substitutions of amino acids, of the capsid protein can cause it to misfold or not form a capsid at all.

Disclosed herein are rAAV variants with increased transduction efficiency compared to wild-type AAV. In some embodiments, transduction efficiency is increased by removing phosphorylation sites that decrease the efficiency of AAV8. SL1.2, disclosed herein, is an rAAV8 variant with sequence modifications to disrupt phosphorylation. Also disclosed herein are rAAV variants that alter tissue tropism compared to wild-type AAV.

In some embodiments, the rAAV variants disclosed herein have increased transduction efficiency in and/or tropism for skeletal muscle compared to wild-type AAV. In some embodiments, the rAAV variants disclosed herein have increased transduction efficiency in and/or tropism for cardiac tissue compared to wild-type AAV. In some embodiments, the rAAV variants disclosed herein have increased transduction efficiency in and/or tropism for tissues of the central nervous system compared to wild-type AAV. In some embodiments, the rAAV variants disclosed herein have increased transduction efficiency in and/or tropism to cardiac tissue but without enhanced transduction in or tropism to CNS tissue. In some embodiments, the rAAV variants disclosed herein have increased transduction efficiency in, and/or tropism to, CNS tissue but without enhanced transduction in, or tropism to, cardiac tissue.

It is therefore useful to use the rAAV variants disclosed herein to deliver a gene to CNS, cardiac and/or skeletal muscle tissue, for example, to treat someone who has a disease or condition of that tissue.

Accordingly, provided herein are rAAV capsid proteins comprising changes (e.g., substitutions), relative to the wild-type AAV8 sequence (e.g., as set forth in SEQ ID NO: 7). In some embodiments, an amino acid substitution in any one of the variant AAV capsid proteins disclosed herein lies in a variable region. In some embodiments, one or more amino acid substitutions fall within recognized variable regions or exposed loops within the AAV capsid sequence. In some embodiments, all amino acid substitutions fall within recognized variable regions or exposed loops within the AAV capsid sequence. It should be understood that any positioning of an amino acid as described herein is with respect to the sequence of the wild-type AAV8 sequence as set forth in SEQ ID NO: 7.

Some non-limiting examples of variant AAV capsid proteins are provided in Table 1. These variants are referred to as SL1.2 (SEQ ID NO: 11), SL1.2L (SEQ ID NO: 15), SL1.2B (SEQ ID NO: 21), SL1.2 LB (SEQ ID NO: 26), SL2 (SEQ ID NO: 12), SL2L (SEQ ID NO: 17), SL2B (SEQ ID NO: 23), SL2LB (SEQ ID NO: 28), SL3 (SEQ ID NO: 13), SL3L (SEQ ID NO: 19), SL3B (SEQ ID NO: 25), and SL2LB (SEQ ID NO: 31).

TABLE 1
Nucleic acid and amino acid sequences for AAV
variants SL1.2, SL1.2L, SL1.2B, SL1.2LB, SL2,
SL2L, SL2B, SL2LB, SL3, SL3L, SL3B, and SL3LB.
Codons shown in all caps in the nucleic acid
sequences are the codon differences from wild-
type AAV8 capsid protein. Underscores indicate
deletions.

| | SEQ | | SEQ | |
|---|---|---|---|---|
| Vari- | ID | Nucleic Acid | ID | Amino Acid |
| ant | NO | Sequence | NO | Sequence |
| SL1.2 | SEQ | atggctgccgatggt | SEQ | MAADGYLPDWLEDNL |
| | ID | tatcttccagattgg | ID | SEGIREWWALKPGAP |
| | NO: | ctcgaggacaacctc | NO: | KPKANQQKQDDGRGL |
| | 1 | tctgagggcattcgc | 11 | VLPGYKYLGPFNGLD |
| | | gagtggtgggcgctg | | KGEPVNAADAAALEH |
| | | aaacctggagccccg | | DKAYDQQLQAGDNPY |
| | | aagcccaaagccaac | | LRYNHADAEFQERLQ |
| | | cagcaaaagcaggac | | EDTSFGGNLGRAVFQ |
| | | gacggccggggtctg | | AKKRVLEPLGLVEEG |
| | | gtgcttcctggctac | | AKTAPGKKRPVEPSP |
| | | aagtacctcggaccc | | QRSPDSSAGIGKSGS |
| | | ttcaacggactcgac | | QPAKKRLNFGQTGDT |
| | | aagggggagcccgtc | | ESVPDPQPIGEPPAA |
| | | aacgcggcggacgca | | PSGVGPNTMAAGGGA |
| | | gcggccctcgagcac | | PMADNNEGADGVGSS |
| | | gacaaggcctacgac | | SGNWHCDSTWLGDRV |
| | | cagcagctgcaggcg | | ITTSTRTWALPTYNN |
| | | ggtgacaatccgtac | | HLYKQISNGTSGGAT |
| | | ctgcggtataaccac | | NDNTYFGYSTPWGYF |
| | | gccgacgccgagttt | | DFNRFHCHFSPRDWQ |
| | | caggagcgtctgcaa | | RLINNNWGFRPKRLS |
| | | gaagatacgtctttt | | FKLFNIQVKEVTQNE |
| | | gggggcaacctcggg | | GTKTIANNLTSTIQV |
| | | cgagcagtcttccag | | FTDSEYQLPYVLGSA |
| | | gccaagaagcgggtt | | HQGCLPPFPADVFMI |
| | | ctcgaacctctcggt | | PQYGYLTLNNGSQAV |
| | | ctggttgaggaaggc | | GRSSFYCLEYFPSQM |
| | | gctaagacggctcct | | LRTGNNFQFTYQFED |
| | | ggaaagaagagaccg | | VPFHSSYAHSQSLDR |
| | | gtagagccatcaccc | | LMNPLIDQYLYFLSR |
| | | cagcgttctccagac | | TQTTGGTANTQQLLF |
| | | tcctctGCGggcatc | | SQGGPNTMANQAKNW |
| | | ggcaagTCGggcTCA | | LPGPCYRQQRVSTVT |
| | | cagcccgccAAAaaa | | GQNNNSNFAWTAGTK |
| | | agactcaattttggt | | YHLNGRNSLANPGIA |
| | | cagactggcgacACA | | MATHKDDEERFFPSN |
| | | gagtcagttccagac | | GILIFGKQNAARDNA |
| | | cctcaacctATCgga | | DYSKVMLTSEEEIKT |
| | | gaacctccagcagcg | | TNPVATEEYGIVADN |
| | | ccctctggtgtggga | | LQQQNAAPQIGTVNS |
| | | cctaatacaatggct | | QGALPGMVWQNRDVY |
| | | gcaggcggtggcgca | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagttcc | | PTTFNQSKLNSFITQ |
| | | tcgggaaattggcat | | YSTGQVSVEIEWELQ |
| | | tgcgattccacatgg | | KENSKRWNPEIQYTS |
| | | ctgggcgacagagtc | | NYYKSTSVDFAVNTE |
| | | atcaccaccagcacc | | GVYSEPRPIGTRFLT |
| | | cgaacctgggccctg | | RNL |
| | | cccacctacaacaac | | |
| | | cacctctacaagcaa | | |
| | | atctccaacgggaca | | |
| | | tcgggaggagccacc | | |
| | | aacgacaacacctac | | |
| | | ttcggctacagcacc | | |
| | | ccctgggggtatttt | | |
| | | gactttaacagattc | | |
| | | cactgccacttttca | | |
| | | ccacgtgactggcag | | |
| | | cgactcatcaacaac | | |
| | | aactggggattccgg | | |
| | | cccaagagactcagc | | |
| | | ttcaagctcttcaac | | |
| | | atccaggtcaaggag | | |
| | | gtcacgcagaatgaa | | |
| | | ggcaccaagaccatc | | |
| | | gccaataacctcacc | | |
| | | agcaccatccaggtg | | |
| | | tttacggactcggag | | |
| | | taccagctgccgtac | | |
| | | gttctcggctctgcc | | |
| | | caccagggctgcctg | | |
| | | cctccgttcccggcg | | |
| | | gacgtgttcatgatt | | |
| | | ccccagtacggctac | | |
| | | ctaacactcaacaac | | |
| | | ggtagtcaggccgtg | | |
| | | ggacgctcctccttc | | |
| | | tactgcctggaatac | | |
| | | tttccttcgcagatg | | |
| | | ctgagaaccggcaac | | |
| | | aacttccagtttact | | |
| | | tacCAGttcgaggac | | |
| | | gtgcctttccacagc | | |
| | | agctacgcccacagc | | |
| | | cagagcttggaccgg | | |
| | | ctgatgaatcctctg | | |
| | | attgaccagtacctg | | |
| | | tacTTCttgtctcgg | | |
| | | actcaaacaacagga | | |
| | | ggcacggcaaatacg | | |
| | | cagCAGctgCTAttc | | |
| | | agccaaggtgggcct | | |
| | | aatacaatggccaat | | |
| | | caggcaaagaactgg | | |
| | | ctgccaggaccctgt | | |
| | | taccgccaacaacgc | | |
| | | gtctcaacgGTAacc | | |
| | | gggcaaaacaacaat | | |
| | | agcaactttgcctgg | | |
| | | actgctgggaccaaa | | |
| | | taccatctgaatgga | | |
| | | agaaattcattggct | | |
| | | aatcctggcatcgct | | |
| | | atggcaacacacaaa | | |
| | | gacgacgaggagcgt | | |
| | | ttttttcccagtaac | | |
| | | gggatcctgattttt | | |
| | | ggcaaacaaaatgct | | |
| | | gccagagacaatgcg | | |
| | | gattacagcAAAgtc | | |
| | | atgctcaccagcgag | | |
| | | gaagaaatcaaaacc | | |
| | | actaaccctgtggct | | |
| | | acagaggaatacggt | | |
| | | atcgtggcagataac | | |
| | | ttgcagcagcaaaac | | |
| | | GCCgctcctcaaatt | | |
| | | ggaactgtcaacagc | | |
| | | cagggggccttaccc | | |
| | | ggtatggtctggcag | | |
| | | aaccgggacgtgtac | | |
| | | ctgcagggtcccatc | | |
| | | tgggccaagattcct | | |
| | | cacacggacggcaac | | |
| | | ttccacccgtctccg | | |
| | | ctgatgggcggcttt | | |
| | | ggcctgaaacatcct | | |
| | | ccgcctcagatcctg | | |
| | | atcaagaacacgcct | | |
| | | gtacctgcggatcct | | |
| | | ccgaccaccttcaac | | |
| | | cagtcaaagctgaac | | |
| | | tctttcatcacgcaa | | |
| | | tacagcaccggacag | | |
| | | gtcagcgtggaaatt | | |
| | | gaatgggagctgcag | | |
| | | aaggaaaacagcaag | | |
| | | cgctggaaccccgag | | |
| | | atccagtacacctcc | | |
| | | aactactacaaatct | | |
| | | acaagtgtggacttt | | |
| | | gctgttaatacagaa | | |
| | | ggcgtgtactctgaa | | |
| | | ccccgccccattggc | | |
| | | acccgtTTCctcacc | | |
| | | cgtaatctgtaa | | |
| SL1.2L | SEQ | atggctgccgatggt | SEQ | MAADGYLPDWLEDNL |
| | ID | tatcttccagattgg | ID | SEGIREWWALKPGAP |
| | NO: | ctcgaggacaacctc | NO: | KPKANQQKQDDGRGL |
| | 14 | tctgagggcattcgc | 15 | VLPGYKYLGPFNGLD |
| | | gagtggtgggcgctg | | KGEPVNAADAAALEH |
| | | aaacctggagccccg | | DKAYDQQLQAGDNPY |
| | | aagcccaaagccaac | | LRYNHADAEFQERLQ |
| | | cagcaaaagcaggac | | EDTSFGGNLGRAVFQ |
| | | gacggccggggtctg | | AKKRVLEPLGLVEEG |
| | | gtgcttcctggctac | | AKTAPGKKRPVEPSP |
| | | aagtacctcggaccc | | QRSPDSSAGIGKSGS |
| | | ttcaacggactcgac | | QPAKKRLNFGQTGDT |
| | | aagggggagcccgtc | | ESVPDPQPIGEPPAA |
| | | aacgcggcggacgca | | PSGVGPNTMAAGGGA |
| | | gcggccctcgagcac | | PMADNNEGADGVGSS |
| | | gacaaggcctacgac | | SGNWHCDSTWLGDRV |
| | | cagcagctgcaggcg | | ITTSTRTWALPTYNN |
| | | ggtgacaatccgtac | | HLYKQISNGTSGGAT |
| | | ctgcggtataaccac | | NDNTYFGYSTPWGYF |
| | | gccgacgccgagttt | | DFNRFHCHFSPRDWQ |
| | | caggagcgtctgcaa | | RLINNNWGFRPKRLS |
| | | gaagatacgtctttt | | FKLFNIQVKEVTQNE |
| | | gggggcaacctcggg | | GTKTIANNLTSTIQV |
| | | cgagcagtcttccag | | FTDSEYQLPYVLGSA |
| | | gccaagaagcgggtt | | HQGCLPPFPADVFMI |
| | | ctcgaacctctcggt | | PQYGYLTLNNGSQAG |
| | | ctggttgaggaaggc | | RSSFYCLEYFPSQML |
| | | gctaagacggctcct | | RTGNNFQFTYQFEDV |
| | | ggaaagaagagaccg | | PFHSSYAHSQSLDRL |
| | | gtagagccatcaccc | | MNPLIDQYLYFLSRT |
| | | cagcgttctccagac | | QTTGGTANTQQLLFS |
| | | tcctctGCGggcatc | | QGGPNTMANQAKNWL |
| | | ggcaagTCGggcTCA | | PGPCYRQQRVSTVTG |
| | | cagcccgccAAAaaa | | QNNISNFAWTAGTKY |
| | | agactcaattttggt | | HLNGRNSLANPGIAM |
| | | cagactggcgacACA | | ATHKDDEERFFPSNG |
| | | gagtcagttccagac | | ILIFGKQNAARDNAD |
| | | cctcaacctATCgga | | YSKVMLTSEEEIKTT |
| | | gaacctccagcagcg | | NPVATEEYGIVADNL |
| | | ccctctggtgtggga | | QQQNAAPQIGTVNSQ |
| | | cctaatacaatggct | | GALPGMVWQNRDVYL |
| | | gcaggcggtggcgca | | QGPIWAKIPHTDGNF |
| | | ccaatggcagacaat | | HPSPLMGGFGLKHPP |
| | | aacgaaggcgccgac | | PQILIKNTPVPADPP |
| | | ggagtgggtagttcc | | TTFNQSKLNSFITQY |
| | | tcgggaaattggcat | | STGQVSVEIEWELQK |
| | | tgcgattccacatgg | | ENSKRWNPEIQYTSN |
| | | ctgggcgacagagtc | | YYKSTSVDFAVNTEG |
| | | atcaccaccagcacc | | VYSEPRPIGTRFLTR |
| | | cgaacctgggccctg | | NL |
| | | cccacctacaacaac | | |
| | | cacctctacaagcaa | | |
| | | atctccaacgggaca | | |
| | | tcgggaggagccacc | | |
| | | aacgacaacacctac | | |
| | | ttcggctacagcacc | | |
| | | ccctgggggtatttt | | |
| | | gactttaacagattc | | |
| | | cactgccacttttca | | |
| | | ccacgtgactggcag | | |
| | | cgactcatcaacaac | | |
| | | aactggggattccgg | | |
| | | cccaagagactcagc | | |
| | | ttcaagctcttcaac | | |
| | | atccaggtcaaggag | | |
| | | gtcacgcagaatgaa | | |
| | | ggcaccaagaccatc | | |
| | | gccaataacctcacc | | |
| | | agcaccatccaggtg | | |
| | | tttacggactcggag | | |
| | | taccagctgccgtac | | |
| | | gttctcggctctgcc | | |
| | | caccagggctgcctg | | |
| | | cctccgttcccggcg | | |
| | | gacgtgttcatgatt | | |
| | | ccccagtacggctac | | |
| | | ctaacactcaacaac | | |
| | | ggtagtcaggccgtg | | |
| | | ggacgctcctccttc | | |
| | | tactgcctggaatac | | |
| | | tttccttcgcagatg | | |
| | | ctgagaaccggcaac | | |
| | | aacttccagtttact | | |
| | | tacCAGttcgaggac | | |
| | | gtgcctttccacagc | | |
| | | agctacgcccacagc | | |
| | | cagagcttggaccgg | | |
| | | ctgatgaatcctctg | | |
| | | attgaccagtacctg | | |
| | | tacTTCttgtctcgg | | |
| | | actcaaacaacagga | | |
| | | ggcacggcaaatacg | | |
| | | cagCAGctgCTAttc | | |
| | | agccaaggtgggcct | | |
| | | aatacaatggccaat | | |
| | | caggcaaagaactgg | | |
| | | ctgccaggaccctgt | | |
| | | taccgccaacaacgc | | |
| | | gtctcaacgGTAacc | | |
| | | gggcaaaacaacATC | | |
| | | agcaactttgcctgg | | |
| | | actgctgggaccaaa | | |
| | | taccatctgaatgga | | |
| | | agaaattcattggct | | |
| | | aatcctggcatcgct | | |
| | | atggcaacacacaaa | | |
| | | gacgacgaggagcgt | | |
| | | ttttttcccagtaac | | |
| | | gggatcctgattttt | | |
| | | ggcaaacaaaatgct | | |
| | | gccagagacaatgcg | | |
| | | gattacagcAAAgtc | | |
| | | atgctcaccagcgag | | |
| | | gaagaaatcaaaacc | | |
| | | actaaccctgtggct | | |
| | | acagaggaatacggt | | |
| | | atcgtggcagataac | | |
| | | ttgcagcagcaaaac | | |
| | | GCCgctcctcaaatt | | |
| | | ggaactgtcaacagc | | |
| | | cagggggccttaccc | | |
| | | ggtatggtctggcag | | |
| | | aaccgggacgtgtac | | |
| | | ctgcagggtcccatc | | |
| | | tgggccaagattcct | | |
| | | cacacggacggcaac | | |
| | | ttccacccgtctccg | | |
| | | ctgatgggcggcttt | | |
| | | ggcctgaaacatcct | | |
| | | ccgcctcagatcctg | | |
| | | atcaagaacacgcct | | |
| | | gtacctgcggatcct | | |
| | | ccgaccaccttcaac | | |
| | | cagtcaaagctgaac | | |
| | | tctttcatcacgcaa | | |
| | | tacagcaccggacag | | |
| | | gtcagcgtggaaatt | | |
| | | gaatgggagctgcag | | |
| | | aaggaaaacagcaag | | |
| | | cgctggaaccccgag | | |
| | | atccagtacacctcc | | |
| | | aactactacaaatct | | |
| | | acaagtgtggacttt | | |
| | | gctgttaatacagaa | | |
| | | ggcgtgtactctgaa | | |
| | | ccccgccccattggc | | |
| | | acccgtTTCctcacc | | |
| | | cgtaatctgtaa | | |
| SL1.2B | SEQ | Atggctgccgatggt | SEQ | MAADGYLPDWLEDNL |
| | ID | tatcttccagattgg | ID | SEGIREWWALKPGAP |
| | NO: | ctcgaggacaacctc | NO: | KPKANQQKQDDGRGL |
| | 20 | tctgagggcattcgc | 21 | VLPGYKYLGPFNGLD |
| | | gagtggtgggcgctg | | KGEPVNAADAAALEH |
| | | aaacctggagccccg | | DKAYDQQLQAGDNPY |
| | | aagcccaaagccaac | | LRYNHADAEFQERLQ |
| | | cagcaaaagcaggac | | EDTSFGGNLGRAVFQ |
| | | gacggccggggtctg | | AKKRVLEPLGLVEEG |
| | | gtgcttcctggctac | | AKTAPGKKRPVEPSP |
| | | aagtacctcggaccc | | QRSPDSSAGIGKSGS |
| | | ttcaacggactcgac | | QPAKKRLNFGQTGDT |
| | | aagggggagcccgtc | | ESVPDPQPIGEPPAA |
| | | aacgcggcggacgca | | PSGVGPNTMAAGGGA |
| | | gcggccctcgagcac | | PMADNNEGADGVGSS |
| | | gacaaggcctacgac | | SGNWHCDSTWLGDRV |
| | | cagcagctgcaggcg | | ITTSTRTWALPTYNN |
| | | ggtgacaatccgtac | | HLYKQISSASTG_AS |
| | | ctgcggtataaccac | | NDNHYFGYSTPWGYF |
| | | gccgacgccgagttt | | DFNRFHCHFSPRDWQ |
| | | caggagcgtctgcaa | | RLINNNWGFRPKRLS |
| | | gaagatacgtctttt | | FKLFNIQVKEVTQNE |
| | | gggggcaacctcggg | | GTKTIANNLTSTIQV |
| | | cgagcagtcttccag | | FTDSEYQLPYVLGSA |
| | | gccaagaagcgggtt | | HQGCLPPFPADVFMI |
| | | ctcgaacctctcggt | | PQYGYLTLNNGSQAV |
| | | ctggttgaggaaggc | | GRSSFYCLEYFPSQM |
| | | gctaagacggctcct | | LRTGNNFQFTYQFED |
| | | ggaaagaagagaccg | | VPFHSSYAHSQSLDR |
| | | gtagagccatcaccc | | LMNPLIDQYLYFLSR |
| | | cagcgttctccagac | | TQTTGGTANTQQLLF |
| | | tcctctGCGggcatc | | SQGGPNTMANQAKNW |
| | | ggcaagTCGggcTCA | | LPGPCYRQQRVSTVT |
| | | cagcccgccAAAaaa | | GQNNNSNFAWTAGTK |
| | | agactcaattttggt | | YHLNGRNSLANPGIA |
| | | cagactggcgacACA | | MATHKDDEERFFPSN |
| | | gagtcagttccagac | | GILIFGKQNAARDNA |
| | | cctcaacctATCgga | | DYSKVMLTSEEEIKT |
| | | gaacctccagcagcg | | TNPVATEEYGIVADN |
| | | ccctctggtgtggga | | LQQQNAAPQIGTVNS |
| | | cctaatacaatggct | | QGALPGMVWQNRDVY |
| | | gcaggcggtggcgca | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagttcc | | PTTFNQSKLNSFITQ |
| | | tcgggaaattggcat | | YSTGQVSVEIEWELQ |
| | | tgcgattccacatgg | | KENSKRWNPEIQYTS |
| | | ctgggcgacagagtc | | NYYKSTSVDFAVNTE |
| | | atcaccaccagcacc | | GVYSEPRPIGTRFLT |
| | | cgaacctgggccctg | | RNL |
| | | cccacctacaacaac | | |
| | | cacctctacaagcaa | | |
| | | atctccTCAGCATCC | | |
| | | ACAgga_gccAGTaa | | |
| | | cgacaacCATtactt | | |
| | | cggctacagcacccc | | |
| | | ctgggggtattttga | | |
| | | ctttaacagattcca | | |
| | | ctgccacttttcacc | | |
| | | acgtgactggcagcg | | |
| | | actcatcaacaacaa | | |
| | | ctggggattccggcc | | |
| | | caagagactcagctt | | |
| | | caagctcttcaacat | | |
| | | ccaggtcaaggaggt | | |
| | | cacgcagaatgaagg | | |
| | | caccaagaccatcgc | | |
| | | caataacctcaccag | | |
| | | caccatccaggtgtt | | |
| | | tacggactcggagta | | |
| | | ccagctgccgtacgt | | |
| | | tctcggctctgccca | | |
| | | ccagggctgcctgcc | | |
| | | tccgttcccggcgga | | |
| | | cgtgttcatgattcc | | |
| | | ccagtacggctacct | | |
| | | aacactcaacaacgg | | |
| | | tagtcaggccgtggg | | |
| | | acgctcctccttcta | | |
| | | ctgcctggaatactt | | |
| | | tccttcgcagatgct | | |
| | | gagaaccggcaacaa | | |
| | | cttccagtttactta | | |
| | | cCAGttcgaggacgt | | |
| | | gcctttccacagcag | | |
| | | ctacgcccacagcca | | |
| | | gagcttggaccggct | | |
| | | gatgaatcctctgat | | |
| | | tgaccagtacctgta | | |
| | | cTTCttgtctcggac | | |
| | | tcaaacaacaggagg | | |
| | | cacggcaaatacgca | | |
| | | gCAGctgCTAttcag | | |
| | | ccaaggtgggcctaa | | |
| | | tacaatggccaatca | | |
| | | ggcaaagaactggct | | |
| | | gccaggaccctgtta | | |
| | | ccgccaacaacgcgt | | |
| | | ctcaacgGTAaccgg | | |
| | | gcaaaacaacaatag | | |
| | | caactttgcctggac | | |
| | | tgctgggaccaaata | | |
| | | ccatctgaatggaag | | |
| | | aaattcattggctaa | | |
| | | tcctggcatcgctat | | |
| | | ggcaacacacaaaga | | |
| | | cgacgaggagcgttt | | |
| | | ttttcccagtaacgg | | |
| | | gatcctgatttttgg | | |
| | | caaacaaaatgctgc | | |
| | | cagagacaatgcgga | | |
| | | ttacagcAAAgtcat | | |
| | | gctcaccagcgagga | | |
| | | agaaatcaaaaccac | | |
| | | taaccctgtggctac | | |
| | | agaggaatacggtat | | |
| | | cgtggcagataactt | | |
| | | gcagcagcaaaacGC | | |
| | | Cgctcctcaaattgg | | |
| | | aactgtcaacagcca | | |
| | | gggggccttacccgg | | |
| | | tatggtctggcagaa | | |
| | | ccgggacgtgtacct | | |
| | | gcagggtcccatctg | | |
| | | ggccaagattcctca | | |
| | | cacggacggcaactt | | |
| | | ccacccgtctccgct | | |
| | | gatgggcggctttgg | | |
| | | cctgaaacatcctcc | | |
| | | gcctcagatcctgat | | |
| | | caagaacacgcctgt | | |
| | | acctgcggatcctcc | | |
| | | gaccaccttcaacca | | |
| | | gtcaaagctgaactc | | |
| | | tttcatcacgcaata | | |
| | | cagcaccggacaggt | | |
| | | cagcgtggaaattga | | |
| | | atgggagctgcagaa | | |
| | | ggaaaacagcaagcg | | |
| | | ctggaaccccgagat | | |
| | | ccagtacacctccaa | | |
| | | ctactacaaatctac | | |
| | | aagtgtggactttgc | | |
| | | tgttaatacagaagg | | |
| | | cgtgtactctgaacc | | |
| | | ccgccccattggcac | | |
| | | ccgtTTCctcacccg | | |
| | | taatctgtaa | | |
| SL1.B2L | SEQ | Atggctgccgatggt | SEQ | MAADGYLPDWLEDNL |
| | ID | tatcttccagattgg | ID | SEGIREWWALKPGAP |
| | NO: | ctcgaggacaacctc | NO: | KPKANQQKQDDGRGL |
| | 26 | tctgagggcattctt | 27 | VLPGYKYLGPFNGLD |
| | | gcgagtggtgggcgc | | KGEPVNAADAAALEH |
| | | tgaaacctggagccc | | DKAYDQQLQAGDNPY |
| | | cgaagcccaaagcca | | LRYNHADAEFQERLQ |
| | | accagcaaaattgca | | EDTSFGGNLGRAVFQ |
| | | ggacgacggccgggg | | AKKRVLEPLGLVEEG |
| | | tctggtgcttcctgg | | AKTAPGKKRPVEPSP |
| | | ctacaagtacctcgg | | QRSPDSSAGIGKSGS |
| | | acccttcaattcgga | | QPAKKRLNFGQTGDT |
| | | ctcgacaagggggag | | ESVPDPQPIGEPPAA |
| | | cccgtcaacgcggcg | | PSGVGPNTMAAGGGA |
| | | gacgcagcggccctc | | PMADNNEGADGVGSS |
| | | gagcattcgacaagg | | SGNWHCDSTWLGDRV |
| | | cctacgaccagcagc | | ITTSTRTWALPTYNN |
| | | tgcaggcgggtgaca | | HLYKQISSASTG_AS |
| | | atccgtacctgcggt | | NDNHYFGYSTPWGYF |
| | | atattaccacgccga | | DFNRFHCHFSPRDWQ |
| | | cgccgagtttcagga | | RLINNNWGFRPKRLS |
| | | gcgtctgcaagaaga | | FKLFNIQVKEVTQNE |
| | | tacgtcttttggggg | | GTKTIANNLTSTIQV |
| | | cattacctcgggcga | | FTDSEYQLPYVLGSA |
| | | gcagtcttccaggcc | | HQGCLPPFPADVFMI |
| | | aagaagcgggttctc | | PQYGYLTLNNGSQAV |
| | | gaacctctcggtctg | | GRSSFYCLEYFPSQM |
| | | gttttgaggaaggcg | | LRTGNNFQFTYQFED |
| | | ctaagacggctcctg | | VPFHSSYAHSQSLDR |
| | | gaaagaagagaccgg | | LMNPLIDQYLYFLSR |
| | | tagagccatcaccct | | TQTTGGTANTQQLLF |
| | | tcagcgttctccaga | | SQGGPNTMANQAKNW |
| | | ctcctctGCGggcat | | LPGPCYRQQRVSTVT |
| | | cggcaagTCGggcTC | | GQNNISNFAWTAGTK |
| | | AcagcccttgccAAA | | YHLNGRNSLANPGIA |
| | | aaaagactcaatttt | | MATHKDDEERFFPSN |
| | | ggtcagactggcgac | | GILIFGKQNAARDNA |
| | | ACAgagtcagttcca | | DYSKVMLTSEEEIKT |
| | | gacttcctcaacctA | | TNPVATEEYGIVADN |
| | | TCggagaacctccag | | LQQQNAAPQIGTVNS |
| | | cagcgccctctggtg | | QGALPGMVWQNRDVY |
| | | tgggacctaatacaa | | LQGPIWAKIPHTDGN |
| | | tttggctgcaggcgg | | FHPSPLMGGFGLKHP |
| | | tggcgcaccaatggc | | PPQILIKNTPVPADP |
| | | agacaataacgaagg | | PTTFNQSKLNSFITQ |
| | | cgccgacggagtttg | | YSTGQVSVEIEWELQ |
| | | ggtagttcctcggga | | KENSKRWNPEIQYTS |
| | | aattggcattgcgat | | NYYKSTSVDFAVNTE |
| | | tccacatggctgggc | | GVYSEPRPIGTRFLT |
| | | gacagagtcatctta | | RNL |
| | | ccaccagcacccgaa | | |
| | | cctgggccctgccca | | |
| | | cctacaacaaccacc | | |
| | | tctacaagcattaat | | |
| | | ctccTCAGCATCCAC | | |
| | | Agga_gccAGTaacg | | |
| | | acaacCATtacttct | | |
| | | tggctacagcacccc | | |
| | | ctgggggtattttga | | |
| | | ctttaacagattcca | | |
| | | ctgccacttttcacc | | |
| | | attcgtgactggcag | | |
| | | cgactcatcaacaac | | |
| | | aactggggattccgg | | |
| | | cccaagagactcagt | | |
| | | tcttcaagctcttca | | |
| | | acatccaggtcaagg | | |
| | | aggtcacgcagaatg | | |
| | | aaggcaccaagactt | | |
| | | catcgccaataacct | | |
| | | caccagcaccatcca | | |
| | | ggtgtttacggactc | | |
| | | ggagtaccagctgtt | | |
| | | ccgtacgttctcggc | | |
| | | tctgcccaccagggc | | |
| | | tgcctgcctccgttc | | |
| | | ccggcggacgtgttt | | |
| | | tcatgattccccagt | | |
| | | acggctacctaacac | | |
| | | tcaacaacggtagtc | | |
| | | aggccgtgggacgtt | | |
| | | ctcctccttctactg | | |
| | | cctggaatactttcc | | |
| | | ttcgcagatgctgag | | |
| | | aaccggcaacaactt | | |
| | | ttccagtttacttac | | |
| | | CAGttcgaggacgtg | | |
| | | cctttccacagcagc | | |
| | | tacgcccacagccat | | |
| | | tgagcttggaccggc | | |
| | | tgatgaatcctctga | | |
| | | ttgaccagtacctgt | | |
| | | acTTCttgtctcggt | | |
| | | tactcaaacaacagg | | |
| | | aggcacggcaaatac | | |
| | | gcagCAGctgCTAtt | | |
| | | cagccaagttgtggg | | |
| | | cctaatacaatggcc | | |
| | | aatcaggcaaagaac | | |
| | | tggctgccaggaccc | | |
| | | tgttaccttgccaac | | |
| | | aacgcgtctcaacgG | | |
| | | TAaccgggcaaaaca | | |
| | | acATCagcaactttg | | |
| | | cctttggactgctgg | | |
| | | gaccaaataccatct | | |
| | | gaatggaagaaattc | | |
| | | attggctaatcctgg | | |
| | | catcgctatggcaac | | |
| | | acacaaagacgacga | | |
| | | ggagcgtttttttcc | | |
| | | cagtaacgggatcct | | |
| | | gttatttttggcaaa | | |
| | | caaaatgctgccaga | | |
| | | gacaatgcggattac | | |
| | | agcAAAgtcatgctc | | |
| | | ttaccagcgaggaag | | |
| | | aaatcaaaaccacta | | |
| | | accctgtggctacag | | |
| | | aggaatacggtatct | | |
| | | tgtggcagataactt | | |
| | | gcagcagcaaaacGC | | |
| | | Cgctcctcaaattgg | | |
| | | aactgtcaacagttc | | |
| | | cagggggccttaccc | | |
| | | ggtatggtctggcag | | |
| | | aaccgggacgtgtac | | |
| | | ctgcagggtcttcca | | |
| | | tctgggccaagattc | | |
| | | ctcacacggacggca | | |
| | | acttccacccgtctc | | |
| | | cgctgatgggttcgg | | |
| | | ctttggcctgaaaca | | |
| | | tcctccgcctcagat | | |
| | | cctgatcaagaacac | | |
| | | gcctgtacctgttcg | | |
| | | gatcctccgaccacc | | |
| | | ttcaaccagtcaaag | | |
| | | ctgaactctttcatc | | |
| | | acgcaatacagctta | | |
| | | ccggacaggtcagcg | | |
| | | tggaaattgaatggg | | |
| | | agctgcagaaggaaa | | |
| | | acagcaagcttgctg | | |
| | | gaaccccgagatcca | | |
| | | gtacacctccaacta | | |
| | | ctacaaatctacaag | | |
| | | tgtggactttttgct | | |
| | | gttaatacagaaggc | | |
| | | gtgtactctgaaccc | | |
| | | cgccccattggcacc | | |
| | | cgtTTCctcttaccc | | |
| | | gtaatctgtaa | | |
| SL2 | SEQ | atggctgccgatggt | SEQ | MAADGYLPDWLEDNL |
| | ID | tatcttccagattgg | ID | SEGIREWWDLKPGAP |
| | NO: | ctcgaggacaacctc | NO: | KPKANQQKQDNGRGL |
| | 3 | tctgagggcattcgc | 12 | VLPGYKYLGPFNGLD |
| | | gagtggtgggacttg | | KGEPVNAADAAALEH |
| | | aaacctggagccccg | | DKAYDQQLKAGDNPY |
| | | aaacccaaagccaac | | LKYNHADAEFQERLQ |
| | | cagcaaaagcaggac | | EDTSFGGNLGRAVFQ |
| | | AACggccggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPAKKRLNFGQTGDS |
| | | aagggggagcccgtc | | ESVPDPQPIGEPPAG |
| | | aacgcggcggacgca | | PSGLGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISNSTSGGST |
| | | ctgAAGtataaccac | | NDNTYFGYSTPWGYF |
| | | gccgacgccgagttt | | DFNRFHCHFSPRDWQ |
| | | caggagcgtctgcaa | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgaaaaag | | NQNNNSNFAWTGATK |
| | | agactcaactttggg | | YHLNGRDSLVNPGIA |
| | | cagactggcgactca | | MASHKEGEERFFPSS |
| | | gagtcagtgcccgac | | GILIFGKQGAGRDNV |
| | | cctcaaccaatcgga | | DYSKVMLTSEEEIKT |
| | | gaaccccccgcaggc | | TNPVATEQYGQVADN |
| | | ccctctggtctggga | | LQQQNAAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccaacAGCact | | |
| | | tcgggaggaagcacc | | |
| | | aacgacaacacctac | | |
| | | ttcggctacagcacc | | |
| | | ccctgggggtatttt | | |
| | | gactttaacagattc | | |
| | | cactgccacttctca | | |
| | | ccacgtgactggcag | | |
| | | cgactcatcaacaac | | |
| | | aactggggattccgg | | |
| | | cccaagAAActcaac | | |
| | | ttcaagctcttcaac | | |
| | | atccaggtcaaggag | | |
| | | gtcacgcagaatgaa | | |
| | | ggcaccaagaccatc | | |
| | | gccaataaccttacc | | |
| | | agcacgattcaggtc | | |
| | | tttacggactcgGAC | | |
| | | taccagctcccgtac | | |
| | | gtcctcggctctgcg | | |
| | | cacGAGggctgcctg | | |
| | | cctccgttcccggcg | | |
| | | gacgtcttcatgatt | | |
| | | cctcagtacgggtac | | |
| | | ctgactctgaacGAT | | |
| | | ggcagtcaggccgtg | | |
| | | ggccgttcctccttc | | |
| | | tactgcctggagtac | | |
| | | tttccttctcaaatg | | |
| | | ctgagaacgggcaac | | |
| | | aactttgagttcagc | | |
| | | taccagtttgaggac | | |
| | | gtgccttttcacagc | | |
| | | agctacgcgcacagc | | |
| | | caaagcctggaccgg | | |
| | | ctgatgaaccccctc | | |
| | | atcgaccagtacctg | | |
| | | tacTTCctgtctcgg | | |
| | | actcagtccacggga | | |
| | | ggtaccgcaggaact | | |
| | | cagcagttgctattt | | |
| | | tctcaggccgggcct | | |
| | | AGCaacatgtcggct | | |
| | | caggccaaaaactgg | | |
| | | ctacccgggccctgc | | |
| | | taccggcagcaacgc | | |
| | | gtctccacgGTAACC | | |
| | | AACcaaaataacaac | | |
| | | agcaactttgcctgg | | |
| | | accggtgccaccaag | | |
| | | tatcatctgaatggc | | |
| | | agagactctctggta | | |
| | | aatcccggtATCgct | | |
| | | atggcaAGCcacaag | | |
| | | GAAGGAgaagagcga | | |
| | | ttttttccgtccagc | | |
| | | ggaATCttaATTttt | | |
| | | gggaaacagggagct | | |
| | | ggaAGAgacaacgtg | | |
| | | gactatagcAAAgtt | | |
| | | atgctaaccagtgag | | |
| | | gaagaaattaaaacc | | |
| | | accaacccagtggcc | | |
| | | acagaacagtacggc | | |
| | | CAAgtggccgataac | | |
| | | ctgcaacagcaaaac | | |
| | | gccgctcctattgta | | |
| | | gggACTgtcaacagt | | |
| | | caaggagccttacct | | |
| | | ggcatggtctggcag | | |
| | | aaccgggacgtgtac | | |
| | | ctgcagggtcctatc | | |
| | | tgggccaagattcct | | |
| | | cacacggacggaaac | | |
| | | tttcatccctcgccg | | |
| | | ctgatgggaggcttt | | |
| | | ggactgaaacacccg | | |
| | | cctcctcagatcctg | | |
| | | attaagaatacacct | | |
| | | gttcccgcggatcct | | |
| | | ccaactaccttcagt | | |
| | | caagctaagctggcg | | |
| | | tcgttcatcacgcag | | |
| | | tacagcaccggacag | | |
| | | gtcagcgtggaaatt | | |
| | | gaatgggagctgcag | | |
| | | aaagaaaacagcaaa | | |
| | | cgctggaacccagag | | |
| | | attcaatacacttcc | | |
| | | aactactacaaatct | | |
| | | acaaatgtggacttt | | |
| | | gctgttaacacaGAA | | |
| | | ggcacttattctgag | | |
| | | cctcgccccatcggc | | |
| | | acccgtTTCctcacc | | |
| | | cgtaatctgtaa | | |
| SL2L | SEQ | atggctgccgatggt | SEQ | MAADGYLPDWLEDNL |
| | ID | tatcttccagattgg | ID | SEGIREWWDLKPGAP |
| | NO: | ctcgaggacaacctc | NO: | KPKANQQKQDNGRGL |
| | 16 | tctgagggcattcgc | 17 | VLPGYKYLGPFNGLD |
| | | gagtggtgggacttg | | KGEPVNAADAAALEH |
| | | aaacctggagccccg | | DKAYDQQLKAGDNPY |
| | | aaacccaaagccaac | | LKYNHADAEFQERLQ |
| | | cagcaaaagcaggac | | EDTSFGGNLGRAVFQ |
| | | AACggccggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPAKKRLNFGQTGDS |
| | | aagggggagcccgtc | | ESVPDPQPIGEPPAG |
| | | aacgcggcggacgca | | PSGLGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISNSTSGGST |
| | | ctgAAGtataaccac | | NDNTYFGYSTPWGYF |
| | | gccgacgccgagttt | | DFNRFHCHFSPRDWQ |
| | | caggagcgtctgcaa | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgaaaaag | | NQNNISNFAWTGATK |
| | | agactcaactttggg | | YHLNGRDSLVNPGIA |
| | | cagactggcgactca | | MASHKEGEERFFPSS |
| | | gagtcagtgcccgac | | GILIFGKQGAGRDNV |
| | | cctcaaccaatcgga | | DYSKVMLTSEEEIKT |
| | | gaaccccccgcaggc | | TNPVATEQYGQVADN |
| | | ccctctggtctggga | | LQQQNAAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccaacAGCact | | |
| | | tcgggaggaagcacc | | |
| | | aacgacaacacctac | | |
| | | ttcggctacagcacc | | |
| | | ccctgggggtatttt | | |
| | | gactttaacagattc | | |
| | | cactgccacttctca | | |
| | | ccacgtgactggcag | | |
| | | cgactcatcaacaac | | |
| | | aactggggattccgg | | |
| | | cccaagAAActcaac | | |
| | | ttcaagctcttcaac | | |
| | | atccaggtcaaggag | | |
| | | gtcacgcagaatgaa | | |
| | | ggcaccaagaccatc | | |
| | | gccaataaccttacc | | |
| | | agcacgattcaggtc | | |
| | | tttacggactcgGAC | | |
| | | taccagctcccgtac | | |
| | | gtcctcggctctgcg | | |
| | | cacGAGggctgcctg | | |
| | | cctccgttcccggcg | | |
| | | gacgtcttcatgatt | | |
| | | cctcagtacgggtac | | |
| | | ctgactctgaacGAT | | |
| | | ggcagtcaggccgtg | | |
| | | ggccgttcctccttc | | |
| | | tactgcctggagtac | | |
| | | tttccttctcaaatg | | |
| | | ctgagaacgggcaac | | |
| | | aactttgagttcagc | | |
| | | taccagtttgaggac | | |
| | | gtgccttttcacagc | | |
| | | agctacgcgcacagc | | |
| | | caaagcctggaccgg | | |
| | | ctgatgaaccccctc | | |
| | | atcgaccagtacctg | | |
| | | tacTTCctgtctcgg | | |
| | | actcagtccacggga | | |
| | | ggtaccgcaggaact | | |
| | | cagcagttgctattt | | |
| | | tctcaggccgggcct | | |
| | | AGCaacatgtcggct | | |
| | | caggccaaaaactgg | | |
| | | ctacccgggccctgc | | |
| | | taccggcagcaacgc | | |
| | | gtctccacgGTAACC | | |
| | | AACcaaaataacATC | | |
| | | agcaactttgcctgg | | |
| | | accggtgccaccaag | | |
| | | tatcatctgaatggc | | |
| | | agagactctctggta | | |
| | | aatcccggtATCgct | | |
| | | atggcaAGCcacaag | | |
| | | GAAGGAgaagagcga | | |
| | | ttttttccgtccagc | | |
| | | ggaATCttaATTttt | | |
| | | gggaaacagggagct | | |
| | | ggaAGAgacaacgtg | | |
| | | gactatagcAAAgtt | | |
| | | atgctaaccagtgag | | |
| | | gaagaaattaaaacc | | |
| | | accaacccagtggcc | | |
| | | acagaacagtacggc | | |
| | | CAAgtggccgataac | | |
| | | ctgcaacagcaaaac | | |
| | | gccgctcctattgta | | |
| | | gggACTgtcaacagt | | |
| | | caaggagccttacct | | |
| | | ggcatggtctggcag | | |
| | | aaccgggacgtgtac | | |
| | | ctgcagggtcctatc | | |
| | | tgggccaagattcct | | |
| | | cacacggacggaaac | | |
| | | tttcatccctcgccg | | |
| | | ctgatgggaggcttt | | |
| | | ggactgaaacacccg | | |
| | | cctcctcagatcctg | | |
| | | atiaagaatacacct | | |
| | | gttcccgcggatcct | | |
| | | ccaactaccttcagt | | |
| | | caagctaagctggcg | | |
| | | tcgttcatcacgcag | | |
| | | tacagcaccggacag | | |
| | | gtcagcgtggaaatt | | |
| | | gaatgggagctgcag | | |
| | | aaagaaaacagcaaa | | |
| | | cgctggaacccagag | | |
| | | attcaatacacttcc | | |
| | | aactactacaaatct | | |
| | | acaaatgtggacttt | | |
| | | gctgttaacacaGAA | | |
| | | ggcacttattctgag | | |
| | | cctcgccccatcggc | | |
| | | acccgtTTCctcacc | | |
| | | cgtaatctgtaa | | |
| SL2B | SEQ | atggctgccgatggt | SEQ | MAADGYLPDWLEDNL |
| | ID | tatcttccagattgg | ID | SEGIREWWDLKPGAP |
| | NO: | ctcgaggacaacctc | NO: | KPKANQQKQDNGRGL |
| | 22 | tctgagggcatCcgc | 23 | VLPGYKYLGPFNGLD |
| | | gagtggtgggacttg | | KGEPVNAADAAALEH |
| | | aaacctggagccccg | | DKAYDQQLKAGDNPY |
| | | aaacccaaagccaac | | LKYNHADAEFQERLQ |
| | | cagcaaaagcaggac | | EDTSFGGNLGRAVFQ |
| | | AACggccggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPAKKRLNFGQTGDS |
| | | aagggggagcccgtc | | ESVPDPQPIGEPPAG |
| | | aacgcggcggacgca | | PSGLGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISSASTG_AS |
| | | ctgAAGtataaccac | | NDNHYFGYSTPWGYF |
| | | gccgacgccgagttt | | DFNRFHCHFSPRDWQ |
| | | caggagcgtctgcaa | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgaaaaag | | NQNNNSNFAWTGATK |
| | | agactcaactttggg | | YHLNGRDSLVNPGIA |
| | | cagactggcgactca | | MASHKEGEERFFPSS |
| | | gagtcagtgcccgac | | GILIFGKQGAGRDNV |
| | | cctcaaccaatcgga | | DYSKVMLTSEEEIKT |
| | | gaaccccccgcaggc | | TNPVATEQYGQVADN |
| | | ccctctggtctggga | | LQQQNAAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccTCAGCATCC | | |
| | | ACAgga_gccAGTaa | | |
| | | cgacaacCATtactt | | |
| | | cggctacagcacccc | | |
| | | ctgggggtattttga | | |
| | | ctttaacagattcca | | |
| | | ctgccacttctcacc | | |
| | | acgtgactggcagcg | | |
| | | actcatcaacaacaa | | |
| | | ctggggattccggcc | | |
| | | caagAAActcaactt | | |
| | | caagctcttcaacat | | |
| | | ccaggtcaaggaggt | | |
| | | cacgcagaatgaagg | | |
| | | caccaagaccatcgc | | |
| | | caataaccttaccag | | |
| | | cacgattcaggtctt | | |
| | | tacggactcgGACta | | |
| | | ccagctcccgtacgt | | |
| | | cctcggctctgcgca | | |
| | | cGAGggctgcctgcc | | |
| | | tccgttcccggcgga | | |
| | | cgtcttcatgattcc | | |
| | | tcagtacgggtacct | | |
| | | gactctgaacGATgg | | |
| | | cagtcaggccgtggg | | |
| | | ccgttcctccttcta | | |
| | | ctgcctggagtactt | | |
| | | tccttctcaaatgct | | |
| | | gagaacgggcaacaa | | |
| | | ctttgagttcagcta | | |
| | | ccagtttgaggacgt | | |
| | | gccttttcacagcag | | |
| | | ctacgcgcacagcca | | |
| | | aagcctggaccggct | | |
| | | gatgaaccccctcat | | |
| | | cgaccagtacctgta | | |
| | | cTTCctgtctcggac | | |
| | | tcagtccacgggagg | | |
| | | taccgcaggaactca | | |
| | | gcagttgctattttc | | |
| | | tcaggccgggcctAG | | |
| | | Caacatgtcggctca | | |
| | | ggccaaaaactggct | | |
| | | acccgggccctgcta | | |
| | | ccggcagcaacgcgt | | |
| | | ctccacgGTAACCAA | | |
| | | Ccaaaataacaatag | | |
| | | caactttgcctggac | | |
| | | cggtgccaccaagta | | |
| | | tcatctgaatggcag | | |
| | | agactctctggtaaa | | |
| | | tcccggtATCgctat | | |
| | | ggcaAGCcacaagGA | | |
| | | AGGAgaagagcgatt | | |
| | | ttttccgtccagcgg | | |
| | | aATCttaATTtttgg | | |
| | | gaaacagggagctgg | | |
| | | aAGAgacaacgtgga | | |
| | | ctatagcAAAgttat | | |
| | | gctaaccagtgagga | | |
| | | agaaattaaaaccac | | |
| | | caacccagtggccac | | |
| | | agaacagtacggcCA | | |
| | | Agtggccgataacct | | |
| | | gcaacagcaaaacgc | | |
| | | cgctcctattgtagg | | |
| | | gACTgtcaacagtca | | |
| | | aggagccttacctgg | | |
| | | catggtctggcagaa | | |
| | | ccgggacgtgtacct | | |
| | | gcagggtcctatctg | | |
| | | ggccaagattcctca | | |
| | | cacggacggaaactt | | |
| | | tcatccctcgccgct | | |
| | | gatgggaggctttgg | | |
| | | actgaaacacccgcc | | |
| | | tcctcagatcctgat | | |
| | | taagaatacacctgt | | |
| | | tcccgcggatcctcc | | |
| | | aactaccttcagtca | | |
| | | agctaagctggcgtc | | |
| | | gttcatcacgcagta | | |
| | | cagcaccggacaggt | | |
| | | cagcgtggaaattga | | |
| | | atgggagctgcagaa | | |
| | | agaaaacagcaaacg | | |
| | | ctggaacccagagat | | |
| | | tcaatacacttccaa | | |
| | | ctactacaaatctac | | |
| | | aaatgtggactttgc | | |
| | | tgttaacacaGAAgg | | |
| | | cacttattctgagcc | | |
| | | tcgccccatcggcac | | |
| | | ccgtTTCctcacccg | | |
| | | taatctgtaa | | |
| SL2LB | SEQ | atggctgccgatggt | SEQ | MAADGYLPDWLEDNL |
| | ID | tatcttccagattgg | ID | SEGIREWWDLKPGAP |
| | NO: | ctcgaggacaacctc | NO: | KPKANQQKQDNGRGL |
| | 28 | tctgagggcattcgc | 29 | VLPGYKYLGPFNGLD |
| | | gagtggtgggacttg | | KGEPVNAADAAALEH |
| | | aaacctggagccccg | | DKAYDQQLKAGDNPY |
| | | aaacccaaagccaac | | LKYNHADAEFQERLQ |
| | | cagcaaaagcaggac | | EDTSFGGNLGRAVFQ |
| | | AACggccggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPAKKRLNFGQTGDS |
| | | aagggggagcccgtc | | ESVPDPQPIGEPPAG |
| | | aacgcggcggacgca | | PSGLGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISSASTG_AS |
| | | ctgAAGtataaccac | | NDNHYFGYSTPWGYF |
| | | gccgacgccgagttt | | DFNRFHCHFSPRDWQ |
| | | caggagcgtctgcaa | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgaaaaag | | NQNNISNFAWTGATK |
| | | agactcaactttggg | | YHLNGRDSLVNPGIA |
| | | cagactggcgactca | | MASHKEGEERFFPSS |
| | | gagtcagtgcccgac | | GILIFGKQGAGRDNV |
| | | cctcaaccaatcgga | | DYSKVMLTSEEEIKT |
| | | gaaccccccgcaggc | | TNPVATEQYGQVADN |
| | | ccctctggtctggga | | LQQQNAAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccTCAGCATCC | | |
| | | ACAgga_gccAGTaa | | |
| | | cgacaacCATtactt | | |
| | | cggctacagcacccc | | |
| | | ctgggggtattttga | | |
| | | ctttaacagattcca | | |
| | | ctgccacttctcacc | | |
| | | acgtgactggcagcg | | |
| | | actcatcaacaacaa | | |
| | | ctggggattccggcc | | |
| | | caagAAActcaactt | | |
| | | caagctcttcaacat | | |
| | | ccaggtcaaggaggt | | |
| | | cacgcagaatgaagg | | |
| | | caccaagaccatcgc | | |
| | | caataaccttaccag | | |
| | | cacgattcaggtctt | | |
| | | tacggactcgGACta | | |
| | | ccagctcccgtacgt | | |
| | | cctcggctctgcgca | | |
| | | cGAGggctgcctgcc | | |
| | | tccgttcccggcgga | | |
| | | cgtcttcatgattcc | | |
| | | tcagtacgggtacct | | |
| | | gactctgaacGATgg | | |
| | | cagtcaggccgtggg | | |
| | | ccgttcctccttcta | | |
| | | ctgcctggagtactt | | |
| | | tccttctcaaatgct | | |
| | | gagaacgggcaacaa | | |
| | | ctttgagttcagcta | | |
| | | ccagtttgaggacgt | | |
| | | gccttttcacagcag | | |
| | | ctacgcgcacagcca | | |
| | | aagcctggaccggct | | |
| | | gatgaaccccctcat | | |
| | | cgaccagtacctgta | | |
| | | cTTCctgtctcggac | | |
| | | tcagtccacgggagg | | |
| | | taccgcaggaactca | | |
| | | gcagttgctattttc | | |
| | | tcaggccgggcctAG | | |
| | | Caacatgtcggctca | | |
| | | ggccaaaaactggct | | |
| | | acccgggccctgcta | | |
| | | ccggcagcaacgcgt | | |
| | | ctccacgGTAACCAA | | |
| | | CcaaaataacATCag | | |
| | | caactttgcctggac | | |
| | | cggtgccaccaagta | | |
| | | tcatctgaatggcag | | |
| | | agactctctggtaaa | | |
| | | tcccggtATCgctat | | |
| | | ggcaAGCcacaagGA | | |
| | | AGGAgaagagcgatt | | |
| | | ttttccgtccagcgg | | |
| | | aATCttaATTtttgg | | |
| | | gaaacagggagctgg | | |
| | | aAGAgacaacgtgga | | |
| | | ctatagcAAAgttat | | |
| | | gctaaccagtgagga | | |
| | | agaaattaaaaccac | | |
| | | caacccagtggccac | | |
| | | agaacagtacggcCA | | |
| | | Agtggccgataacct | | |
| | | gcaacagcaaaacgc | | |
| | | cgctcctattgtagg | | |
| | | gACTgtcaacagtca | | |
| | | aggagccttacctgg | | |
| | | catggtctggcagaa | | |
| | | ccgggacgtgtacct | | |
| | | gcagggtcctatctg | | |
| | | ggccaagattcctca | | |
| | | cacggacggaaactt | | |
| | | tcatccctcgccgct | | |
| | | gatgggaggctttgg | | |
| | | actgaaacacccgcc | | |
| | | tcctcagatcctgat | | |
| | | taagaatacacctgt | | |
| | | tcccgcggatcctcc | | |
| | | aactaccttcagtca | | |
| | | agctaagctggcgtc | | |
| | | gttcatcacgcagta | | |
| | | cagcaccggacaggt | | |
| | | cagcgtggaaattga | | |
| | | atgggagctgcagaa | | |
| | | agaaaacagcaaacg | | |
| | | ctggaacccagagat | | |
| | | tcaatacacttccaa | | |
| | | ctactacaaatctac | | |
| | | aaatgtggactttgc | | |
| | | tgttaacacaGAAgg | | |
| | | cacttattctgagcc | | |
| | | tcgccccatcggcac | | |
| | | ccgtTTCctcacccg | | |
| | | taatctgtaa | | |
| SL3 | SEQ | atggctgccgatggt | SEQ | MAADGYLPDWLEDNL |
| | ID | tatcttccagattgg | ID | SEGIREWWALKPGAP |
| | NO: | ctcgaggacaacctc | NO: | QPKANQQKQDNARGL |
| | 5 | tctgagggcattcgc | 13 | VLPGYKYLGPFNGLD |
| | | gagtggtggGCTttg | | KGEPVNAADAAALEH |
| | | aaacctggagccccg | | DKAYDQQLKAGDNPY |
| | | CAAcccaaagccaac | | LKYNHADAEFQERLK |
| | | cagcaaaagcaggac | | EDTSFGGNLGRAVFQ |
| | | AACGCTcggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPARKRLNFGQTGDT |
| | | aagggggagcccgtc | | ESVPDPQPLGEPPAA |
| | | aacgcggcggacgca | | PSGVGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISNSTSGGST |
| | | ctgAAGtataaccac | | NDNTYFGYSTPWGYF |
| | | gccgacgccgagttt | | DFNRFHCHFSPRDWQ |
| | | caggagcgtctgAAA | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgAGAaag | | NQNNNSNFAWTGATK |
| | | agactcaactttggg | | YHLNGRDSLVNPGIA |
| | | cagactggcgacACA | | MASHKEGEERFFPSS |
| | | gagtcagtgcccgac | | GILIFGKQGAGRDNV |
| | | cctcaaccaCTCgga | | DYSKVMLTSEEEIKT |
| | | gaaccccccgcaGCC | | TNPVATEEYGQVADN |
| | | ccctctggtGTGgga | | LQSANTAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccaacAGCact | | |
| | | tcgggaggaagcacc | | |
| | | aacgacaacacctac | | |
| | | ttcggctacagcacc | | |
| | | ccctgggggtatttt | | |
| | | gactttaacagattc | | |
| | | cactgccacttctca | | |
| | | ccacgtgactggcag | | |
| | | cgactcatcaacaac | | |
| | | aactggggattccgg | | |
| | | cccaagAAActcaac | | |
| | | ttcaagctcttcaac | | |
| | | atccaggtcaaggag | | |
| | | gtcacgcagaatgaa | | |
| | | ggcaccaagaccatc | | |
| | | gccaataaccttacc | | |
| | | agcacgattcaggtc | | |
| | | tttacggactcgGAC | | |
| | | taccagctcccgtac | | |
| | | gtcctcggctctgcg | | |
| | | cacGAGggctgcctg | | |
| | | cctccgttcccggcg | | |
| | | gacgtcttcatgatt | | |
| | | cctcagtacgggtac | | |
| | | ctgactctgaacGAT | | |
| | | ggcagtcaggccgtg | | |
| | | ggccgttcctccttc | | |
| | | tactgcctggagtac | | |
| | | tttccttctcaaatg | | |
| | | ctgagaacgggcaac | | |
| | | aactttgagttcagc | | |
| | | taccagtttgaggac | | |
| | | gtgccttttcacagc | | |
| | | agctacgcgcacagc | | |
| | | caaagcctggaccgg | | |
| | | ctgatgaaccccctc | | |
| | | atcgaccagtacctg | | |
| | | tacTTCctgtctcgg | | |
| | | actcagtccacggga | | |
| | | ggtaccgcaggaact | | |
| | | cagcagttgctattt | | |
| | | tctcaggccgggcct | | |
| | | AGCaacatgtcggct | | |
| | | caggccaaaaactgg | | |
| | | ctacccgggccctgc | | |
| | | taccggcagcaacgc | | |
| | | gtctccacgGTAACC | | |
| | | AACcaaaataacaac | | |
| | | agcaactttgcctgg | | |
| | | accggtgccaccaag | | |
| | | tatcatctgaatggc | | |
| | | agagactctctggta | | |
| | | aatcccggtATCgct | | |
| | | atggcaAGCcacaag | | |
| | | GAAGGAgaagagcga | | |
| | | ttttttccgtccagc | | |
| | | ggaATCttaATTttt | | |
| | | gggaaacagggagct | | |
| | | ggaAGAgacaacgtg | | |
| | | gactatagcAAAgtt | | |
| | | atgctaaccagtgag | | |
| | | gaagaaattaaaacc | | |
| | | accaacccagtggcc | | |
| | | acagaaGAAtacggc | | |
| | | CAAgtggccgataac | | |
| | | ctgcaaAGTGCCaac | | |
| | | ACGgctcctattgta | | |
| | | gggACTgtcaacagt | | |
| | | caaggagccttacct | | |
| | | ggcatggtctggcag | | |
| | | aaccgggacgtgtac | | |
| | | ctgcagggtcctatc | | |
| | | tgggccaagattcct | | |
| | | cacacggacggaaac | | |
| | | tttcatccctcgccg | | |
| | | ctgatgggaggcttt | | |
| | | ggactgaaacacccg | | |
| | | cctcctcagatcctg | | |
| | | attaagaatacacct | | |
| | | gttcccgcggatcct | | |
| | | ccaactaccttcagt | | |
| | | caagctaagctggcg | | |
| | | tcgttcatcacgcag | | |
| | | tacagcaccggacag | | |
| | | gtcagcgtggaaatt | | |
| | | gaatgggagctgcag | | |
| | | aaagaaaacagcaaa | | |
| | | cgctggaacccagag | | |
| | | attcaatacacttcc | | |
| | | aactactacaaatct | | |
| | | acaaatgtggacttt | | |
| | | gctgttaacacaGAA | | |
| | | ggcacttattctgag | | |
| | | cctcgccccatcggc | | |
| | | acccgtTTCctcacc | | |
| | | cgtaatctgtaa | | |
| SL3L | SEQ | atggctgccgatggt | SEQ | MAADGYLPDWLEDNL |
| | ID | tatcttccagattgg | ID | SEGIREWWALKPGAP |
| | NO: | ctcgaggacaacctc | NO: | QPKANQQKQDNARGL |
| | 18 | tctgagggcattcgc | 19 | VLPGYKYLGPFNGLD |
| | | gagtggtggGCTttg | | KGEPVNAADAAALEH |
| | | aaacctggagccccg | | DKAYDQQLKAGDNPY |
| | | CAAcccaaagccaac | | LKYNHADAEFQERLK |
| | | cagcaaaagcaggac | | EDTSFGGNLGRAVFQ |
| | | AACGCTcggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPARKRLNFGQTGDT |
| | | aagggggagcccgtc | | ESVPDPQPLGEPPAA |
| | | aacgcggcggacgca | | PSGVGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISNSTSGGST |
| | | ctgAAGtataaccac | | NDNTYFGYSTPWGYF |
| | | gccgacgccgagttt | | DFNRFHCHFSPRDWQ |
| | | caggagcgtctgAAA | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgAGAaag | | NQNNISNFAWTGATK |
| | | agactcaactttggg | | YHLNGRDSLVNPGIA |
| | | cagactggcgacACA | | MASHKEGEERFFPSS |
| | | gagtcagtgcccgac | | GILIFGKQGAGRDNV |
| | | cctcaaccaCTCgga | | DYSKVMLTSEEEIKT |
| | | gaaccccccgcaGCC | | TNPVATEEYGQVADN |
| | | ccctctggtGTGgga | | LQSANTAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccaacAGCact | | |
| | | tcgggaggaagcacc | | |
| | | aacgacaacacctac | | |
| | | ttcggctacagcacc | | |
| | | ccctgggggtatttt | | |
| | | gactttaacagattc | | |
| | | cactgccacttctca | | |
| | | ccacgtgactggcag | | |
| | | cgactcatcaacaac | | |
| | | aactggggattccgg | | |
| | | cccaagAAActcaac | | |
| | | ttcaagctcttcaac | | |
| | | atccaggtcaaggag | | |
| | | gtcacgcagaatgaa | | |
| | | ggcaccaagaccatc | | |
| | | gccaataaccttacc | | |
| | | agcacgattcaggtc | | |
| | | tttacggactcgGAC | | |
| | | taccagctcccgtac | | |
| | | gtcctcggctctgcg | | |
| | | cacGAGggctgcctg | | |
| | | cctccgttcccggcg | | |
| | | gacgtcttcatgatt | | |
| | | cctcagtacgggtac | | |
| | | ctgactctgaacGAT | | |
| | | ggcagtcaggccgtg | | |
| | | ggccgttcctccttc | | |
| | | tactgcctggagtac | | |
| | | tttccttctcaaatg | | |
| | | ctgagaacgggcaac | | |
| | | aactttgagttcagc | | |
| | | taccagtttgaggac | | |
| | | gtgccttttcacagc | | |
| | | agctacgcgcacagc | | |
| | | caaagcctggaccgg | | |
| | | ctgatgaaccccctc | | |
| | | atcgaccagtacctg | | |
| | | tacTTCctgtctcgg | | |
| | | actcagtccacggga | | |
| | | ggtaccgcaggaact | | |
| | | cagcagttgctattt | | |
| | | tctcaggccgggcct | | |
| | | AGCaacatgtcggct | | |
| | | caggccaaaaactgg | | |
| | | ctacccgggccctgc | | |
| | | taccggcagcaacgc | | |
| | | gtctccacgGTAACC | | |
| | | AACcaaaataacATC | | |
| | | agcaactttgcctgg | | |
| | | accggtgccaccaag | | |
| | | tatcatctgaatggc | | |
| | | agagactctctggta | | |
| | | aatcccggtATCgct | | |
| | | atggcaAGCcacaag | | |
| | | GAAGGAgaagagcga | | |
| | | ttttttccgtccagc | | |
| | | ggaATCttaATTttt | | |
| | | gggaaacagggagct | | |
| | | ggaAGAgacaacgtg | | |
| | | gactatagcAAAgtt | | |
| | | atgctaaccagtgag | | |
| | | gaagaaattaaaacc | | |
| | | accaacccagtggcc | | |
| | | acagaaGAAtacggc | | |
| | | CAAgtggccgataac | | |
| | | ctgcaaAGTGCCaac | | |
| | | ACGgctcctattgta | | |
| | | gggACTgtcaacagt | | |
| | | caaggagccttacct | | |
| | | ggcatggtctggcag | | |
| | | aaccgggacgtgtac | | |
| | | ctgcagggtcctatc | | |
| | | tgggccaagattcct | | |
| | | cacacggacggaaac | | |
| | | tttcatccctcgccg | | |
| | | ctgatgggaggcttt | | |
| | | ggactgaaacacccg | | |
| | | cctcctcagatcctg | | |
| | | attaagaatacacct | | |
| | | gttcccgcggatcct | | |
| | | ccaactaccttcagt | | |
| | | caagctaagctggcg | | |
| | | tcgttcatcacgcag | | |
| | | tacagcaccggacag | | |
| | | gtcagcgtggaaatt | | |
| | | gaatgggagctgcag | | |
| | | aaagaaaacagcaaa | | |
| | | cgctggaacccagag | | |
| | | attcaatacacttcc | | |
| | | aactactacaaatct | | |
| | | acaaatgtggacttt | | |
| | | gctgttaacacaGAA | | |
| | | ggcacttattctgag | | |
| | | cctcgccccatcggc | | |
| | | acccgtTTCctcacc | | |
| | | cgtaatctgtaa | | |
| SL3B | SEQ | atggctgccgatggt | SEQ | MAADGYLPDWLEDNL |
| | ID | tatcttccagattgg | ID | SEGIREWWALKPGAP |
| | NO: | ctcgaggacaacctc | NO: | QPKANQQKQDNARGL |
| | 24 | tctgagggcattcgc | 25 | VLPGYKYLGPFNGLD |
| | | gagtggtggGCTttg | | KGEPVNAADAAALEH |
| | | aaacctggagccccg | | DKAYDQQLKAGDNPY |
| | | CAAcccaaagccaac | | LKYNHADAEFQERLK |
| | | cagcaaaagcaggac | | EDTSFGGNLGRAVFQ |
| | | AACGCTcggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPARKRLNFGQTGDT |
| | | aagggggagcccgtc | | ESVPDPQPLGEPPAA |
| | | aacgcggcggacgca | | PSGVGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISSASTG_AS |
| | | ctgAAGtataaccac | | NDNHYFGYSTPWGYF |
| | | gccgacgccgagttt | | DFNRFHCHFSPRDWQ |
| | | caggagcgtctgAAA | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgAGAaag | | NQNNNSNFAWTGATK |
| | | agactcaactttggg | | YHLNGRDSLVNPGIA |
| | | cagactggcgacACA | | MASHKEGEERFFPSS |
| | | gagtcagtgcccgac | | GILIFGKQGAGRDNV |
| | | cctcaaccaCTCgga | | DYSKVMLTSEEEIKT |
| | | gaaccccccgcaGCC | | TNPVATEEYGQVADN |
| | | ccctctggtGTGgga | | LQSANTAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccTCAGCATCC | | |
| | | ACAgga_gccAGTaa | | |
| | | cgacaacCATtactt | | |
| | | cggctacagcacccc | | |
| | | ctgggggtattttga | | |
| | | ctttaacagattcca | | |
| | | ctgccacttctcacc | | |
| | | acgtgactggcagcg | | |
| | | actcatcaacaacaa | | |
| | | ctggggattccggcc | | |
| | | caagAAActcaactt | | |
| | | caagctcttcaacat | | |
| | | ccaggtcaaggaggt | | |
| | | cacgcagaatgaagg | | |
| | | caccaagaccatcgc | | |
| | | caataaccttaccag | | |
| | | cacgattcaggtctt | | |
| | | tacggactcgGACta | | |
| | | ccagctcccgtacgt | | |
| | | cctcggctctgcgca | | |
| | | cGAGggctgcctgcc | | |
| | | tccgttcccggcgga | | |
| | | cgtcttcatgattcc | | |
| | | tcagtacgggtacct | | |
| | | gactctgaacGATgg | | |
| | | cagtcaggccgtggg | | |
| | | ccgttcctccttcta | | |
| | | ctgcctggagtactt | | |
| | | tccttctcaaatgct | | |
| | | gagaacgggcaacaa | | |
| | | ctttgagttcagcta | | |
| | | ccagtttgaggacgt | | |
| | | gccttttcacagcag | | |
| | | ctacgcgcacagcca | | |
| | | aagcctggaccggct | | |
| | | gatgaaccccctcat | | |
| | | cgaccagtacctgta | | |
| | | cTTCctgtctcggac | | |
| | | tcagtccacgggagg | | |
| | | taccgcaggaactca | | |
| | | gcagttgctattttc | | |
| | | tcaggccgggcctAG | | |
| | | Caacatgtcggctca | | |
| | | ggccaaaaactggct | | |
| | | acccgggccctgcta | | |
| | | ccggcagcaacgcgt | | |
| | | ctccacgGTAACCAA | | |
| | | Ccaaaataacaatag | | |
| | | caactttgcctggac | | |
| | | cggtgccaccaagta | | |
| | | tcatctgaatggcag | | |
| | | agactctctggtaaa | | |
| | | tcccggtATCgctat | | |
| | | ggcaAGCcacaagGA | | |
| | | AGGAgaagagcgatt | | |
| | | ttttccgtccagcgg | | |
| | | aATCttaATTtttgg | | |
| | | gaaacagggagctgg | | |
| | | aAGAgacaacgtgga | | |
| | | ctatagcAAAgttat | | |
| | | gctaaccagtgagga | | |
| | | agaaattaaaaccac | | |
| | | caacccagtggccac | | |
| | | agaaGAAtacggcCA | | |
| | | Agtggccgataacct | | |
| | | gcaaAGTGCCaacAC | | |
| | | Ggctcctattgtagg | | |
| | | gACTgtcaacagtca | | |
| | | aggagccttacctgg | | |
| | | catggtctggcagaa | | |
| | | ccgggacgtgtacct | | |
| | | gcagggtcctatctg | | |
| | | ggccaagattcctca | | |
| | | cacggacggaaactt | | |
| | | tcatccctcgccgct | | |
| | | gatgggaggctttgg | | |
| | | actgaaacacccgcc | | |
| | | tcctcagatcctgat | | |
| | | taagaatacacctgt | | |
| | | tcccgcggatcctcc | | |
| | | aactaccttcagtca | | |
| | | agctaagctggcgtc | | |
| | | gttcatcacgcagta | | |
| | | cagcaccggacaggt | | |
| | | cagcgtggaaattga | | |
| | | atgggagctgcagaa | | |
| | | agaaaacagcaaacg | | |
| | | ctggaacccagagat | | |
| | | tcaatacacttccaa | | |
| | | ctactacaaatctac | | |
| | | aaatgtggactttgc | | |
| | | tgttaacacaGAAgg | | |
| | | cacttattctgagcc | | |
| | | tcgccccatcggcac | | |
| | | ccgtTTCctcacccg | | |
| | | taatctgtaa | | |
| SL3LB | SEQ | atggctgccgatggt | SEQ | MAADGYLPDWLEDNL |
| | ID | tatcttccagattgg | ID | SEGIREWWALKPGAP |
| | NO: | ctcgaggacaacctc | NO: | QPKANQQKQDNARGL |
| | 30 | tctgagggcattcgc | 31 | VLPGYKYLGPFNGLD |
| | | gagtggtggGCTttg | | KGEPVNAADAAALEH |
| | | aaacctggagccccg | | DKAYDQQLKAGDNPY |
| | | CAAcccaaagccaac | | LKYNHADAEFQERLK |
| | | cagcaaaagcaggac | | EDTSFGGNLGRAVFQ |
| | | AACGCTcggggtctg | | AKKRVLEPLGLVEEG |
| | | gtgcttcctggctac | | AKTAPGKKRPVEPSP |
| | | aagtacctcggaccc | | QRSPDSSAGIGKSGA |
| | | ttcaacggactcgac | | QPARKRLNFGQTGDT |
| | | aagggggagcccgtc | | ESVPDPQPLGEPPAA |
| | | aacgcggcggacgca | | PSGVGSNTMAAGGGA |
| | | gcggccctcgagcac | | PMADNNEGADGVGSA |
| | | gacaaggcctacgac | | SGNWHCDSTWLGDRV |
| | | cagcagctcaaagcg | | ITTSTRTWALPTYNN |
| | | ggtgacaatccgtac | | HLYKQISSASTG_AS |
| | | ctgAAGtataaccac | | NDNHYFGYSTPWGYF |
| | | gccgacgccgagttt | | DFNRFHCHFSPRDWQ |
| | | caggagcgtctgAAA | | RLINNNWGFRPKKLN |
| | | gaagatacgtctttt | | FKLFNIQVKEVTQNE |
| | | gggggcaacctcggg | | GTKTIANNLTSTIQV |
| | | cgagcagtcttccag | | FTDSDYQLPYVLGSA |
| | | gccaagaagcgggtt | | HEGCLPPFPADVFMI |
| | | ctcgaacctctcggt | | PQYGYLTLNDGSQAV |
| | | ctggttgaggaaggc | | GRSSFYCLEYFPSQM |
| | | gctaagacggctcct | | LRTGNNFEFSYQFED |
| | | ggaaagaagagaccg | | VPFHSSYAHSQSLDR |
| | | gtagagccatcaccc | | LMNPLIDQYLYFLSR |
| | | cagcgttctccagac | | TQSTGGTAGTQQLLF |
| | | tcctctGCGggcatc | | SQAGPSNMSAQAKNW |
| | | ggcaagTCGggcGCA | | LPGPCYRQQRVSTVT |
| | | cagcccgcgAGAaag | | NQNNISNFAWTGATK |
| | | agactcaactttggg | | YHLNGRDSLVNPGIA |
| | | cagactggcgacACA | | MASHKEGEERFFPSS |
| | | gagtcagtgcccgac | | GILIFGKQGAGRDNV |
| | | cctcaaccaCTCgga | | DYSKVMLTSEEEIKT |
| | | gaaccccccgcaGCC | | TNPVATEEYGQVADN |
| | | ccctctggtGTGgga | | LQSANTAPIVGTVNS |
| | | tctAATacaatggct | | QGALPGMVWQNRDVY |
| | | gcaggcggtggcgct | | LQGPIWAKIPHTDGN |
| | | ccaatggcagacaat | | FHPSPLMGGFGLKHP |
| | | aacgaaggcgccgac | | PPQILIKNTPVPADP |
| | | ggagtgggtagtGCC | | PTTFSQAKLASFITQ |
| | | tcaggaaattggcat | | YSTGQVSVEIEWELQ |
| | | tgcgattccacatgg | | KENSKRWNPEIQYTS |
| | | ctgggcgacagagtc | | NYYKSTNVDFAVNTE |
| | | atcaccaccagcacc | | GTYSEPRPIGTRFLT |
| | | cgaacctgggccctc | | RNL |
| | | cccacctacaacaac | | |
| | | cacctctacaagcaa | | |
| | | atctccTCAGCATCC | | |
| | | ACAgga_gccAGTaa | | |
| | | cgacaacCATtactt | | |
| | | cggctacagcacccc | | |
| | | ctgggggtattttga | | |
| | | ctttaacagattcca | | |
| | | ctgccacttctcacc | | |
| | | acgtgactggcagcg | | |
| | | actcatcaacaacaa | | |
| | | ctggggattccggcc | | |
| | | caagAAActcaactt | | |
| | | caagctcttcaacat | | |
| | | ccaggtcaaggaggt | | |
| | | cacgcagaatgaagg | | |
| | | caccaagaccatcgc | | |
| | | caataaccttaccag | | |
| | | cacgattcaggtctt | | |
| | | tacggactcgGACta | | |
| | | ccagctcccgtacgt | | |
| | | cctcggctctgcgca | | |
| | | cGAGggctgcctgcc | | |
| | | tccgttcccggcgga | | |
| | | cgtcttcatgattcc | | |
| | | tcagtacgggtacct | | |
| | | gactctgaacGATgg | | |
| | | cagtcaggccgtggg | | |
| | | ccgttcctccttcta | | |
| | | ctgcctggagtactt | | |
| | | tccttctcaaatgct | | |
| | | gagaacgggcaacaa | | |
| | | ctttgagttcagcta | | |
| | | ccagtttgaggacgt | | |
| | | gccttttcacagcag | | |
| | | ctacgcgcacagcca | | |
| | | aagcctggaccggct | | |
| | | gatgaaccccctcat | | |
| | | cgaccagtacctgta | | |
| | | cTTCctgtctcggac | | |
| | | tcagtccacgggagg | | |
| | | taccgcaggaactca | | |
| | | gcagttgctattttc | | |
| | | tcaggccgggcctAG | | |
| | | Caacatgtcggctca | | |
| | | ggccaaaaactggct | | |
| | | acccgggccctgcta | | |
| | | ccggcagcaacgcgt | | |
| | | ctccacgGTAACCAA | | |
| | | CcaaaataacATCag | | |
| | | caactttgcctggac | | |
| | | cggtgccaccaagta | | |
| | | tcatctgaatggcag | | |
| | | agactctctggtaaa | | |
| | | tcccggtATCgctat | | |
| | | ggcaAGCcacaagGA | | |
| | | AGGAgaagagcgatt | | |
| | | ttttccgtccagcgg | | |
| | | aATCttaATTtttgg | | |
| | | gaaacagggagctgg | | |
| | | aAGAgacaacgtgga | | |
| | | ctatagcAAAgttat | | |
| | | gctaaccagtgagga | | |
| | | agaaattaaaaccac | | |
| | | caacccagtggccac | | |
| | | agaaGAAtacggcCA | | |
| | | Agtggccgataacct | | |
| | | gcaaAGTGCCaacAC | | |
| | | Ggctcctattgtagg | | |
| | | gACTgtcaacagtca | | |
| | | aggagccttacctgg | | |
| | | catggtctggcagaa | | |
| | | ccgggacgtgtacct | | |
| | | gcagggtcctatctg | | |
| | | ggccaagattcctca | | |
| | | cacggacggaaactt | | |
| | | tcatccctcgccgct | | |
| | | gatgggaggctttgg | | |
| | | actgaaacacccgcc | | |
| | | tcctcagatcctgat | | |
| | | taagaatacacctgt | | |
| | | tcccgcggatcctcc | | |
| | | aactaccttcagtca | | |
| | | agctaagctggcgtc | | |
| | | gttcatcacgcagta | | |
| | | cagcaccggacaggt | | |
| | | cagcgtggaaattga | | |
| | | atgggagctgcagaa | | |
| | | agaaaacagcaaacg | | |
| | | ctggaacccagagat | | |
| | | tcaatacacttccaa | | |
| | | ctactacaaatctac | | |
| | | aaatgtggactttgc | | |
| | | tgttaacacaGAAgg | | |
| | | cacttattctgagcc | | |
| | | tcgccccatcggcac | | |
| | | ccgtTTCctcacccg | | |
| | | taatctgtaa | | |

It is to be understood that any one of the variant recombinant AAV (e.g., rAAV8) capsid proteins disclosed herein may have any one single amino acid substitution described herein, or any combination of amino acid substitutions described herein.

For example, in some embodiments SL1.2, SL2, and SL3 are further mutated. In some embodiments, SL1.2, SL2, and SL3 are further mutated to alter the tropism of the variants to certain tissues (e.g., heart, neural, or other muscle tissue). In some embodiments, the further mutations of SL1.2, SL2, and SL3 improve the uptake of SL1.2, SL2, and/or SL3 in the tissue of interest (e.g., heart, neural, or other muscle tissue). In some embodiments, the further mutations of SL1.2, SL2, and SL3 reduce the uptake of SL1.2, SL2, and/or SL3 in the tissue of non-interest (e.g., neural or liver tissue).

In some embodiments, the capsid region spanning amino acids Glu578 to Gly596 is further mutated in SL1.2, SL2, and/or SL3. In some embodiments, one amino acid is further mutated in SL1.2, SL2, and/or SL3: Asparagine 500 to Isoleucine. In some embodiments, the further mutations introduced into any of SL1.2, SL2, and SL3 incorporate those described in Pulicherla, et al., Mol. Ther. 19:6, 1070-78 (2011). In some embodiments, the Asparagine 500 to Isoleucine mutation in SL1.2, SL2, and/or SL3 results in reduced liver tissue tropism.

In some embodiments, the Asparagine 500 to Isoleucine mutation in SL1.2 results in an AAV variant protein having the amino acid sequence of SEQ ID NO: 15 (“SL1.2L”). In some embodiments, the Asparagine 500 to Isoleucine mutation in SL1.2 results in an AAV variant protein that is encoded by a nucleic acid having the sequence of SEQ ID NO: 14. In some embodiments, the Asparagine 500 to Isoleucine mutation in SL2 results in an AAV variant protein having the amino acid sequence of SEQ ID NO: 17 (“SL2L”). In some embodiments, the Asparagine 500 to Isoleucine mutation in SL2 results in an AAV variant protein that is encoded by a nucleic acid having the sequence of SEQ ID NO: 16. In some embodiments, the Asparagine 500 to Isoleucine mutation in SL3 results in an AAV variant protein having the amino acid sequence of SEQ ID NO: 19 (“SL3L”). In some embodiments, the Asparagine 500 to Isoleucine mutation in SL3 results in an AAV variant protein that is encoded by a nucleic acid having that sequence of SEQ ID NO: 18.

In some embodiments, seven amino acids are further mutated in SL1.2: Asparagine 263 to Serine; Glycine 264 to Alanine; Threonine 265 to Serine; Serine 266 to Threonine; deletion of Glycine 268; Threonine 270 to Serine; and Threonine 274 to Histidine (“the seven amino acid mutations in SL1.2”). In some embodiments, eight amino acids are further mutated in SL2: Asparagine 263 to Serine; Serine 264 to Alanine; Threonine 265 to Serine; Serine 266 to Threonine; deletion of Glycine 268; Serine 269 to Alanine; Threonine 270 to Serine; and Threonine 274 to Histidine (“the eight amino acid mutations in SL2”). In some embodiments, eight amino acids are further mutated in SL3: Asparagine 263 to Serine; Serine 264 to Alanine; Threonine 265 to Serine; Serine 266 to Threonine; deletion of Glycine 268; Serine 269 to Alanine; Threonine 270 to Serine; and Threonine 274 to Histidine (“the eight amino acid mutations in SL3”). In some embodiments, the further mutations introduced into any of SL1.2, SL2, and SL3 incorporate those described in Albright, et al., Mol. Ther. 26:2, 510-23 (2018). In some embodiments, the seven amino acid mutations in SL1.2 or the eight amino acid mutations in SL2 and/or SL3 results in reduced brain tissue tropism.

In some embodiments, the seven amino acid mutations in SL1.2 result in an AAV variant protein having the amino acid sequence of SEQ ID NO: 21 (“SL1.2B”). In some embodiments, the seven amino acid mutations in SL1.2 result in an AAV variant protein that is encoded by a nucleic acid having that sequence of SEQ ID NO: 20. In some embodiments, the eight amino acid mutations in SL2 result in an AAV variant protein having the amino acid sequence of SEQ ID NO: 23 (“SL2B”). In some embodiments, the eight amino acid mutations in SL2 result in an AAV variant protein that is encoded by a nucleic acid having that sequence of SEQ ID NO: 22. In some embodiments, the eight amino acid mutations in SL3 result in an AAV variant protein having the amino acid sequence of SEQ ID NO: 25 (“SL3B”). In some embodiments, the eight amino acid mutations in SL3 result in an AAV variant protein that is encoded by a nucleic acid having that sequence of SEQ ID NO: 24.

In some embodiments, eight amino acids are further mutated in SL1.2: Asparagine 500 to Isoleucine; Asparagine 263 to Serine; Glycine 264 to Alanine; Threonine 265 to Serine; Serine 266 to Threonine; deletion of Glycine 268; Threonine 270 to Serine; and Threonine 274 to Histidine (“the seven amino acid mutations in SL1.2”). In some embodiments, the eight amino acid mutations in SL1.2 results in reduced liver and brain tissue tropism. In some embodiments, the eight amino acid mutations in SL1.2 result in an AAV variant protein having the amino acid sequence of SEQ ID NO: 27 (“SL1.2 LB”). In some embodiments, the eight amino acid mutations in SL1.2 result in an AAV variant protein that is encoded by a nucleic acid having that sequence of SEQ ID NO: 26.

In some embodiments, nine amino acids are further mutated in SL2: Asparagine 500 to Isoleucine; Asparagine 263 to Serine; Serine 264 to Alanine; Threonine 265 to Serine; Serine 266 to Threonine; deletion of Glycine 268; Serine 269 to Alanine; Threonine 270 to Serine; and Threonine 274 to Histidine (“the nine amino acid mutations in SL2”). In some embodiments, the nine amino acid mutations in SL2 results in reduced liver and brain tissue tropism. In some embodiments, the nine amino acid mutations in SL2 result in an AAV variant protein having the amino acid sequence of SEQ ID NO: 29 (“SL2LB”). In some embodiments, the nine amino acid mutations in SL2 result in an AAV variant protein that is encoded by a nucleic acid having that sequence of SEQ ID NO: 28.

In some embodiments, nine amino acids are further mutated in SL3: Asparagine 500 to Isoleucine; Asparagine 263 to Serine; Serine 264 to Alanine; Threonine 265 to Serine; Serine 266 to Threonine; deletion of Glycine 268; Serine 269 to Alanine; Threonine 270 to Serine; and Threonine 274 to Histidine (“the nine amino acid mutations in SL3”). In some embodiments, the nine amino acid mutations in SL3 results in reduced liver and brain tissue tropism. In some embodiments, the nine amino acid mutations in SL3 result in an AAV variant protein having the amino acid sequence of SEQ ID NO: 31 (“SL3LB”). In some embodiments, the nine amino acid mutations in SL3 result in an AAV variant protein that is encoded by a nucleic acid having that sequence of SEQ ID NO: 30.

Contemplated herein are also variant rAAV capsid proteins of serotypes other than serotype 8. In some embodiments, any one of the amino acid changes described herein are in a variable region of the capsid protein of a serotype other than serotype 8 that is homologous to the variable region of AAV8 (e.g., 1, 2, 3, 3B, 4, 5, 6, 7, 9, 10, 11, 12, or 13). In some embodiments, a variant rAAV capsid could be made on a background of the cap gene of one serotype delivered with the rep gene of a different serotype.

### Recombinant AAV Vectors

As used herein, the term “vector” may refer to a nucleic acid vector (e.g., a plasmid or recombinant viral genome), a wild-type AAV genome, or a virus that comprises a viral genome. In some embodiments, the term “vector” may refer to a viral particle, such as an AAV viral particle.

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners. Either a longer or shorter intron can be excised resulting in the formation of two isoforms of mRNAs: a ~2.3 kb- and a ~2.6 kb-long mRNA isoform. The capsid forms a supramolecular assembly of approximately 60 individual capsid protein subunits into a non-enveloped, T-1 icosahedral lattice capable of protecting the AAV genome. A mature AAV capsid is composed of VP1, VP2, and VP3 (molecular masses of approximately 87, 73, and 62 kDa respectively) in a ratio of about 1:1:10.

Recombinant AAV (rAAV) particles may comprise a recombinant nucleic acid vector (hereafter referred to as “rAAV vector”), which may comprise at a minimum: (a) one or more heterologous nucleic acid regions comprising a sequence encoding a transgene; and (b) one or more regions comprising sequences that facilitate the integration of the heterologous nucleic acid region (optionally with the one or more nucleic acid regions comprising a sequence that facilitates expression) into the genome of the subject. In some embodiments, the sequences facilitating the integration of the heterologous nucleic acid region (optionally with the one or more nucleic acid regions comprising a sequence that facilitates expression) into the genome of the subject are inverted terminal repeat (ITR) sequences (e.g., wild-type ITR sequences or engineered ITR sequences) flanking the one or more nucleic acid regions (e.g., heterologous nucleic acid regions).

In some embodiments, the rAAV nucleic acid vector comprises one or more transgenes comprising a sequence encoding a protein or polypeptide of interest operably linked to a promoter, wherein the one or more transgenes are flanked on each side with an ITR sequence. In some embodiments, the nucleic acid vector further comprises a region encoding a Rep protein as described herein, either contained within the region flanked by ITRs or outside the region or nucleic acid) operably linked to a promoter. The ITR sequences may be derived from any AAV serotype (e.g., 1, 2, 3, 4, 5, 6, 7, 8, 9, or 10) or may be derived from more than one serotype. In some embodiments, the ITR sequences are derived from AAV2 or AAV6 serotypes. In some embodiments, a first serotype provided herein is not an AAV2 or AAV8 serotype. In some embodiments, the ITR sequences of the first serotype are derived from AAV3, AAV5 or AAV6. In some embodiments, the ITR sequences are derived from AAV2, AAV3, AAV5 or AAV6. In some embodiments, the ITR sequences are the same serotype as the capsid (e.g., AAV6 ITR sequences and AAV6 capsid, etc.). In some embodiments, the ITR sequences are derived from AAVrh.10 serotype.

In some embodiments, a recombinant AAV (e.g., rAAV8) particle containing any one of the variant rAAV capsid proteins disclosed herein comprises ITRs and/or rep ORF of serotype 8. In some embodiments, a rAAV particle is a pseudotyped rAAV particle, which comprises (a) a capsid comprised of capsid proteins containing modifications described herein made on a serotype 8 background, and (b) a nucleic acid vector comprising ITRs from another serotype (e.g., AAV1, AAV2, AAV3, AAV4, AAV5, AAV6, AAV7, AAV9, or AAV10).

The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the serotype of the capsid proteins of the recombinant virus. In some embodiments, the rAAV particle is rAAV6 or rAAV9. Non-limiting examples of derivatives and pseudotypes include AAVrh.10, AAVrh.74, AAV2/1, AAV2/5, AAV2/6, AAV2/8, AAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). In particular embodiments, the capsid of any of the herein disclosed rAAV particles is of the AAVrh.10 serotype. In some embodiments, the capsid is of the AAV2/6 serotype. In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 @ Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference). In some embodiments, the rAAV comprises a pTR-UF-11 plasmid backbone, which is a plasmid that contains AAV2 ITRs. This plasmid is commercially available from the American Type Culture Collection (ATCC MBA-331).

Provided herein are variant recombinant AAV (e.g., rAAV8) particles. In some embodiments, a particle is an empty particle (e.g., one that does not contain a nucleic acid vector comprising a gene of interest). In some embodiments, an AAV8 particle contains a nucleic acid vector comprising a gene of interest. As used herein, “a gene of interest” is a gene that encodes an RNA or protein of interest.

Thus, in some embodiments, the rAAV vector comprises one or more regions comprising a sequence that facilitates expression of the gene of interest, e.g., expression control sequences operably linked to the nucleic acid. Numerous such sequences are known in the art. Non-limiting examples of expression control sequences include promoters, insulators, silencers, response elements, introns, enhancers, initiation sites, internal ribosome entry sites (IRES) termination signals, and poly(A) signals. Any combination of such control sequences is contemplated herein (e.g., a promoter and an enhancer). In some embodiments, the rAAV vectors comprise a promoter that is operably linked to the coding sequence of the gene of interest and facilitates expression of the gene of interest. A “promoter”, as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). In some embodiments, one or more promoters may be operably linked to a coding nucleotide sequence in the heterologous nucleic acid. A promoter is considered to be “operably linked” to a sequence of nucleic acid that it regulates when the promoter is in a correct functional location and orientation relative to the sequence such that the promoter controls and/or regulates (e.g., to control (“drive”) transcriptional initiation and/or expression of) that sequence. A promoter may be a constitutive promoter, tissue-specific promoter, an inducible promoter, or a synthetic promoter.

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A cytomegalovirus (CMV) promoters. Non-limiting examples of constitutive mammalian promoters include various housekeeping gene promoters, as exemplified by the β-actin promoter (e.g., chicken β-actin promoter) and human elongation factor-1α (EF-1α) promoter. In some embodiments, chimeric viral/mammalian promoters may include a chimeric CMV/chicken beta actin (CBA, CB or CAG) promoters.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. In some embodiments, the promoter may be a tissue-specific promoter. A “tissue-specific promoter”, as used herein, refers to promoters that can only function in a specific type of tissue, e.g., the heart. Thus, a “tissue-specific promoter” is not able to drive the expression of the transgenes in other types of tissues. In some embodiments, it may be beneficial to combine a variant rAAV particle as disclosed herein, with a promoter that also targets the same cells, tissue, or organ as the variant rAAV particle. For example, for variants with improved cardiac tropism, the use of a cardiac troponin T promoter to achieve cardiac-specific expression would be appropriate.

Promoters that may be used in accordance with the present disclosure may in some embodiments comprise any promoter that can drive the expression of the transgenes in the heart of the subject. In some embodiments, the promoter that may be used in accordance with the present disclosure is a cardiac-restricted promoter. For example, promoter is a cardiac-restricted promoter selected from cardiac troponin C, cardiac troponin I, and cardiac troponin T (cTnT).

Alternatively, the promoter may be, without limitation, a promoter from one of the following genes: α-myosin heavy chain gene, 6-myosin heavy chain gene, myosin light chain 2v (MLC-2v) gene, myosin light chain 2a gene, CARP gene, cardiac α-actin gene, cardiac m2 muscarinic acetylcholine gene, ANF, cardiac troponin C, cardiac troponin I, cardiac troponin T (cTnT), cardiac sarcoplasmic reticulum Ca-ATPase gene, skeletal α-actin; or an artificial cardiac promoter derived from MLC-2v gene.

In some embodiments, the rAAV vectors of the present disclosure further comprise a polyadenylation (pA) signal. Eukaryotic mRNAs are typically transcribed as a precursor mRNA. The precursor mRNA is processed to generate the mature mRNA, including a polyadenylation process. The process of polyadenylation begins as the transcription of a gene terminates. The 3′-most segment of the newly made precursor mRNA is first cleaved off by a set of proteins. These proteins then synthesize the poly(A) tail at the RNA's 3′ end. The cleavage site typically contains the polyadenylation signal, e.g., AAUAAA. The poly(A) tail is important for the nuclear export, translation, and stability of mRNA.

In some embodiments, the rAAV vectors of the present disclosure comprise at least, in order from 5′ to 3′, a first adeno-associated virus (AAV) inverted terminal repeat (ITR) sequence, a promoter operably linked to a first transgene, an IRES operably linked to a second transgene, a polyadenylation signal, and a second AAV inverted terminal repeat (ITR) sequence.

In some embodiments, the rAAV is circular. In some embodiments, the rAAV vector is linear. In some embodiments, the rAAV vector is single-stranded. In some embodiments, the rAAV vector is double-stranded. In some embodiments, the rAAV vector is a self-complementary rAAV vector. Any rAAV vector described herein may be encapsidated by a viral capsid, such as an AAV6 capsid or any other serotype (e.g., a serotype that is of the same serotype as the ITR sequences).

A protein of interest may be a detectable marker or a therapeutic protein. A detectable marker may be a molecule that can be visualized (e.g., using a naked eye or under a microscope). In some embodiments, the detectable marker is a fluorescent molecule, a bioluminescent molecule, or a molecule that provides color (e.g., β-galactosidase, β-lactamases, β-glucuronidase and spheroidine). In some embodiments, a detectable marker is a fluorescent protein or functional peptide or functional polypeptide thereof.

In some embodiments, a gene of interest encodes a therapeutic protein and is referred to as a “therapeutic gene.” A therapeutic gene may provide a therapeutic effect in a cell, tissue or organ to which it is delivered. In some embodiments, a therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, an anticoagulant, a nuclease, guide RNA or other nucleic acid or protein for gene editing. In some embodiments, a gene of interest encodes a therapeutic RNA, e.g., a small interfering RNA.

In some embodiments, a therapeutic gene (e.g., a gene of interest) is a cardioprotective gene. Several cardioprotective genes are known, including heme oxygenase-1. This protein degrades the pro-oxidant heme and generates carbon monoxide and antioxidant bilirubin, conferring myocardial protection from ischemia/reperfusion injury. Franz et al., Circ. Res. 73:629-638, 1993. In inflammatory diseases, HO-1 is increased as a cytoprotective gene. However, it is usually insufficient in amount to stop the inflammation. The vigilant vector could provide an amplified amount of HO-1 when reduced oxygen indicates a need for HO-1. Another example of a cardioprotective gene is superoxide dismutase, which protects heart tissue from super oxide radicals generated during ischemia-reperfusion. Chen et al., Circulation 94:11412-11417, 1996; and Woo et al., Circulation 98:11255-11260, 1998. Genes that provide a protective effect from other cardiac disease states, such as heart degeneration and failure, may also be used in vectors of the invention. An example of a gene that improves cardiac function is phospholamban (PLN). The PLN gene product regulates the strength of each heartbeat and is known to malfunction in heart failure. Zvaritch et al., J. Biol. Chem. 275:14985-14991, 2000. Any suitable cardioprotective gene that provides a therapeutically effective level of protection may be used within vectors of the invention.

In some embodiments, a cardioprotective gene is a gene that is critical for signaling pathways in cardioprotection. In some embodiments, a cardioprotective gene is any one of: protectomiRs (e.g., microRNA 125b*), ZAC1 transcription factor, phospholamban (PLN), pro-inflammatory genes such as cyclooxygenase (COX)-2 and inducible nitric oxide synthase (iNOS), antioxidant enzymes such as hemoxygenase (HO)-1, extracellular and manganese superoxidase dismutases (ec-SOD and Mg-SOD), heat shock proteins (HSPs), growth factors such as insulin like growth factor (IGF)-1 and hepatocyte growth factor (HGF), antiapoptotic proteins such as Bcl-2 and Bcl-xL, pro-apoptotic proteins such as FasL, Bcl-2, Bax, caspase-3 and p53, and proangiogenic genes such as TGFbeta, sphingosine kinase 1 (SPK1), apoptosis repressor with caspase recruitment domain (ARC), wild type cardiac troponin T, wild type cardiac myosin binding protein C, myosin light chains (either regulatory or essential), PI3K-Akt, and/or S100 variants.

S100 family proteins that may be used in accordance to the present disclosure include, without limitation, S100A1, S100A2, S100A3, S100A4, S100A5, S100A6, S100A7 (e.g., psoriasin), S100A8 (e.g., calgranulin A), S100A9 (e.g., calgranulin B), S100A10, S100A11, S100A12 (e.g., calgranulin C), S100A13, S100A14, S100A15 (e.g., koebnerisin), S100A16, S100B, S100P, and S100Z, or variants thereof.

In some embodiments, the S100 family protein may be S100 calcium-binding protein A1 (S100A1). In some embodiments, the S100A1 is cardiac S100A1 (cS100A1) or a variant thereof. The cS100A1 protein is a regulator of myocardial contractility. cS100A1 protein levels are reduced in right ventricular hypertrophied tissue in a model of pulmonary hypertension. Further, S100A1 is a regulator of the genetic program underlying cardiac hypertrophy, in that S100A1 inhibits alpha1 adrenergic stimulation of hypertrophic genes, including MYH7, ACTA1 and S100B.

In cardiomyocytes, S100A1 regulates the calcium-controlled network of SR, sarcomeric, and mitochondrial function through modulation of ryanodine receptor 2 (RYR2), sarco/endoplasmic reticulum Ca2+-ATPase (SERCA), titin, and mitochondrial F1-ATPase activity. As a result, cardiomyocytes and hearts with increased S100A1 expression show increased systolic and diastolic performance, a result of improved Ca2+ transient amplitudes resulting from augmented SR Ca2+ load and subsequent systolic Ca2+ release together with decreased diastolic SR Ca2+ leak and enhanced Ca2+ re-sequestration. Concurrently, S100A1 increases mitochondrial high-energy phosphate production and thus coordinates the energy supply with the increased adenosine 5′-triphosphate (ATP) demand by the enhanced cardiomyocyte Ca2+ turnover. Reduced S100A1 expression in cardiomyocytes is associated with reduced contractile function, corroborating the pathophysiological significance of this protein.

In some embodiments, the S100A1 cDNA (transgene) sequence of the polynucleotides of any of the disclosed rAAV vectors has 100% identity to a naturally-occurring human-derived S100A1 sequence. In other embodiments, the S100A1 cDNA sequence has at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to a naturally-occurring S100A1 sequence.

In some embodiments, the S100A1 cDNA sequence is codon-optimized for expression in human cells. In other embodiments, the S100A1 cDNA sequence is codon-optimized for expression in canine cells.

Aspects of the present disclosure provide compositions and methods that include the delivery of a gene encoding an apoptotic inhibitor (e.g., an anti-apoptotic agent). Illustrative examples of apoptotic inhibitors include fink, p35, crmA, Bcl-2, Bcl-XL, Mcl-1, E1B-19K from adenovirus, as well as antagonists of pro-apoptotic agents (e.g., antisense, ribozymes, antibodies, etc.). In some embodiments, the apoptotic inhibitor is cardiac Apoptosis Repressor with Caspase Recruitment Domain (ARC), or a variant thereof. In other embodiments, the apoptotic inhibitor is cardiac ARC or a variant thereof. In some embodiments, it may be desirable to deliver an S100 family protein and the apoptotic inhibitor separately. In certain embodiments, a gene encoding the S100 family protein is delivered concurrently or sequentially with one or more small molecule apoptotic inhibitors. Other exemplary small-molecule apoptotic inhibitors include c-Myc inhibitors, Bax inhibitors, p53 inhibitors, tBid inhibitors, caspase inhibitors, and inhibitors of pro-apoptotic BCL-2 family members.

The cARC is an apoptotic regulatory protein expressed almost exclusively in myogenic cells. It contains a caspase recruitment domain (CARD) through which it blocks the activation of some initiator caspases. ARC also blocks caspase-independent events associated with apoptosis. Apoptosis caused by acute ischemia and subsequent ventricular remodeling is implicated as a mediator of heart failure. Although post-ischemic heart failure may have multiple causes, recent attention has been directed toward understanding the contribution of apoptosis or programmed cell death. Apoptosis is characterized by preservation of mitochondrial and sarcolemmal membranes, nuclear chromatin condensation, and phagocytosis by macrophages or neighboring cells without triggering an inflammatory response. The activation of apoptosis is known to occur through mechanisms involving caspases, a family of cysteine proteases that are synthesized as inactive precursors and proteolytically cleaved into their active form. ARC is able the block the activation of apoptosis by blocking the caspases.

In particular embodiments, the cARC cDNA sequence is codon-optimized for expression in human cells. In other embodiments, the cARC cDNA sequence is codon-optimized for expression in canine cells.

In some embodiments, a gene of interest encodes an antisense molecule. A number of antisense molecules that confer a cardioprotective effect are known, including antisense to angiotensin II type-1 receptor (Yang et al., Circulation 96:922-926, 1997; and Yang et al., Circ. Res. 83:552-559, 1998), antisense to adrenergic beta-1 receptor (Chen et al., Pharmacol. Exp. Ther. 294:722-727, 2000), and antisense to angiotensin-converting enzyme that has been shown to protect rat hearts from ischemia-reperfusion (Chen et al., Pharmacol. Exp. Ther. 294:722-727, 2000). In some embodiments, the antisense molecule is antisense to angiotensin II type-1 receptor, antisense to adrenergic beta-1 receptor, or antisense to angiotensin-converting enzyme.

In some embodiments, a nucleic acid vector comprised in an rAAV (e.g., an rAAV8) particle comprises one or more of the following: (a) one or more heterologous nucleic acid regions comprising gene of interest, and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, a nucleic acid vector in a rAAV particle comprises one or more nucleic acid regions comprising a control sequence that facilitates expression of the heterologous nucleic acid region (e.g., a promoter). In some embodiments, a nucleic acid vector in a recombinant AAV (e.g., rAAV8) particle comprises one or more nucleic acid regions comprising a sequence that facilitates integration of the heterologous nucleic acid region (optionally with the one or more nucleic acid regions comprising a sequence that facilitates expression) into the genome of the subject.

In some embodiments, the AAV is a fully constituted AAV containing the cap gene as modified by the invention, or an AAV capsid as modified by the invention carrying a transgene with the capsid protein supplied in trans. In some embodiments, empty capsids comprising the modified capsid proteins disclosed could be used to pre-treat prior to use of filled AAV capsids to further enhance transduction.

Described in Table 1 are exemplary rAAV capsid protein or related gene sequences of the present disclosure. The rAAV capsid proteins illustrated in Table 1 comprise the amino acid sequences set forth as SEQ ID NOs: 11-13, 15, 17, 19, 21, 23, 25, 27, 29, and 31.

The rAAV capsid proteins of the disclosure may comprise amino sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 11-13, 15, 17, 19, 21, 23, 25, 27, 29, and 31.

The rAAV capsid proteins illustrated in Table 1 are encoded by polynucleotides having the sequences set forth as SEQ ID NOs: 1, 3, 5, 14, 16, 18, 20, 22, 24, 26, 28, and 30. The rAAV capsid proteins of the disclosure may be encoded by nucleic acid sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 1, 3, 5, 14, 16, 18, 20, 22, 24, 26, 28, and 30.

As a practical matter, whether any particular nucleic acid molecule is at least 80%, 85%, 90%, 95%, 96%, 97%, 98%, or 99% identical to, for instance, the nucleotide sequence of a transgene, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

In some embodiments, any of the disclosed rAAV amino acid vector sequences comprise truncations at the 5′ or 3′ end relative to the sequences of any one of SEQ ID NOs: 11-13, 15, 17, 19, 21, 23, 25, 27, 29, and 31. In some embodiments, any of the rAAV vectors comprise an amino acid sequence that differs from the sequence of any one of SEQ ID NOs: 11-13, 15, 17, 19, 21, 23, 25, 27, 29, and 31 by 1, 2, 3, 4, 5, 6, 7, 8, 9, 10, 11, 12, 13, 14, 15, 16, 17, 18, or more than 18 amino acids. In some embodiments, any of the disclosed rAAV nucleic acid vector sequences comprise truncations at the 5′ or 3′ end relative to the sequences of any one of SEQ ID NOs: 1, 3, 5, 14, 16, 18, 20, 22, 24, 26, 28, and 30. In some embodiments, any of the rAAV vectors are encoded by a nucleic acid sequence that differs from the sequence of any one of SEQ ID NOs: 1, 3, 5, 14, 16, 18, 20, 22, 24, 26, 28, and 30 by 1, 2, 3, 4, 5, 6, 7, 8, 9, 10, 11, 12, 13, 14, 15, 16, 17, 18, or more than 18 nucleic acids.

Method of Making rAAV Particles

Further provided herein are methods of making rAAV particles. The rAAV particles comprise a viral capsid and an rAAV vector as described herein, which is encapsidated by the viral capsid. Various methods of producing rAAV particles and nucleic acid vectors are known in the art and are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). In some embodiments, a vector (e.g., a plasmid) comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP region as described herein), and transfected into a recombinant cells, called helper or producer cells, such that the nucleic acid vector is packaged or encapsidated inside the capsid and subsequently purified.

Non-limiting examples of mammalian helper cells include HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). A non-limiting example of an insect helper cells is Sf9 cells (see, e.g., ATCC® CRL-1711™). A helper cell may comprise rep and/or cap genes that encode the Rep protein and/or Cap proteins. In some embodiments, the packaging is performed in vitro (e.g., outside of a cell).

In some embodiments, a nucleic acid vector (e.g., a plasmid) containing the gene of interest is combined with one or more helper plasmids, e.g., that contain a rep gene of a first serotype and a cap gene of the same serotype or a different serotype, and transfected into helper cells such that the rAAV particle is packaged. In some embodiments, the one or more helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA. Helper plasmids, and methods of making such plasmids, are known in the art and commercially available (see, e.g., pDF6, pRep, pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also know and available. For example, Addgene Catalog #37825-AAV8.T includes a helper plasmid hat comprises rep and the wild-type AAV8 cap gene (https://www.addgene.org/37825/#37825-AAV8).

ITR sequences and plasmids containing ITR sequences are known in the art and are commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 @ Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

Genbank reference numbers for sequences of AAV serotypes 1, 2, 3, 3B, 4, 5, 6, 7, 8, 9, 10, 11, 12, and 13 are listed in patent publication WO2012064960, which is incorporated herein by reference in its entirety.

A non-limiting method of rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. As an example, HEK293 cells (available from ATCC®) are transfected via CaPO4-mediated transfection, lipids or polymeric molecules such as Polyethylenimine (PEI) with the helper plasmid(s) and a plasmid containing a nucleic acid vector. The HEK293 cells are then incubated for at least 60 hours to allow for rAAV particle production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus and herpesvirus.

Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation.

Methods for large-scale production of AAV using a herpesvirus-based system are also known. See for example, Clement et al. (Hum Gene Ther. 2009, 20 (8): 796-806). Methods of producing exosome-associated AAV, which can be more resistant to neutralizing anti-AAV antibodies, are also known (Hudry et al., Gene Ther. 2016, 23(4):380-92; Macguire et al., Mol Ther. 2012, 20 (5): 960-71).

Methods for producing and using pseudotyped rAAV vectors are also known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

### Compositions

The present disclosure is also directed to compositions comprising one or more of the disclosed rAAV particles or preparations. In some embodiments, the rAAV preparation comprises an rAAV particle comprising a rAAV vector containing ITRs of a first serotype (e.g., AAV3, AAV5, AAV6, or AAV9) and capsid proteins encapsidating the rAAV vector. In some embodiments, the capsid proteins are of the first serotype (e.g., AAV3, AAV5, AAV6, or AAV9). In some embodiments, the preparation has at least a four-fold higher transduction efficiency (e.g., in a human hepatocellular carcinoma cell line, such as Huh7) compared to a preparation prepared using a rAAV vector containing AAV2 ITRs.

As described herein, such compositions may further comprise a pharmaceutical excipient, buffer, or diluent, and may be formulated for administration to host cell ex vivo or in situ in an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a human subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders as described herein.

Various formulations have been developed to facilitate rAAV particle use. For example, for administration of an injectable aqueous solution of rAAV particles, the solution may be suitably buffered, if necessary, and the liquid diluent first rendered isotonic with sufficient saline or glucose. In some embodiments, a composition as provided herein comprises a plurality of any one of the variant rAAV particles disclosed herein. In some embodiments, a composition comprises pluralities of more than one of the variant rAAV particles disclosed herein. In some embodiments, “administering” or “administration” means providing a material to a subject in a manner that is pharmacologically useful.

Accordingly, in some embodiments, a composition of variant rAAV particles comprises a pharmaceutically acceptable carrier. The term “carrier” refers to a diluent, adjuvant, excipient, or vehicle with which the rAAV particle is administered. Such pharmaceutical carriers can be sterile liquids (e.g., water, oils, saline solutions, aqueous dextrose and glycerol solutions), suspending agents, preserving agents (e.g., methyl-, ethyl-, and propyl-hydroxy-benzoates), and pH adjusting agents (such as inorganic and organic acids and bases). In some embodiments, carriers include buffered saline solutions (e.g., phosphate buffered saline, HEPES-buffered saline). USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

In some embodiments, a composition comprising any one of the rAAV particles disclosed herein comprises Balanced Salt Solution (BSS) supplemented with 0.014% Tween 20 (polysorbate 20).

Typically, compositions may contain at least about 0.1% of the therapeutic agent (e.g., rAAV particle) or more, although the percentage of the active ingredient(s) may be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms of rAAV particle compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. In some embodiments, the form is sterile. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

Preparation of compositions for administration to a subject are known in the art. For example, a dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

rAAV Gene Therapy for Heart Diseases

The rAAV vectors, rAAV particles, or the composition comprising the rAAV particles of the present disclosure, may be used for gene therapy for heart diseases in a human subject in need thereof. Examples of heart disease that may be treated using the methods and compositions of the present disclosure include, but are not limited to, cardiomyopathy and acute ischemia. In some embodiments, the heart cardiomyopathy is hypertrophic cardiomyopathy or dilated cardiomyopathy. Heart failure caused by cardiomyopathy or other heart diseases, comprise two components, calcium handling dysfunction and apoptosis. The rAAV vectors, particles, and compositions comprising the rAAV particles may be used for treatment of such heart failures when administered to a subject in need thereof, e.g., via vascular delivery into the coronary arteries and/or direct injection to the heart. The rAAV vectors, particles, and compositions comprising the rAAV particles drive the concurrent expression of cS100A1 protein and ARC proteins in the cardiomyocytes of the subject. S100A1 improves aspects of calcium handling, including normalization of sarcoplasmic reticular calcium transients leading to normalization of contractile function. ARC will block apoptosis initiated by mitochondrial and nonmitochondrial mechanisms (such as stretch-induced apoptosis), as well as improve mitochondrial function. Thus, the synergistic benefits of the two proteins expressed by the transgenes of the present disclosure can lead to better long-term therapeutic outcomes by targeting both aspects of cardiomyopathy.

### Methods of Transducing Cells

Any one of the rAAV particles, or compositions comprising any one of the rAAV particles disclosed herein can be used to transduce a cell, tissue or organ. In some embodiments, a cell, tissue or organ that is transduced using any one of the variant rAAV particles disclosed herein is transduced with a gene of interest that may be a therapeutic gene or one that is desired to study. In some embodiments, a cell, tissue or organ is transduced in an in vitro setting wherein the cell, tissue or organ is incubated or perfused with a media. A cell may be one of many cells cultured under certain conditions, or part of an organ that is harvested, part of an organoid, or an organism.

In some embodiments, a cell, tissue or organ is transduced in vivo, for example, for the purposes of treating a disease. In some embodiments, such a rAAV particle comprises a gene of interest that encodes a therapeutic protein or RNA.

In some embodiments, a composition comprising any one or more of the variant rAAV particles disclosed herein is provided to cells. In some embodiments, a composition comprising any one or more of the variant rAAV particles disclosed herein is provided to tissue in the CNS, to skeletal muscle, or to cardiac tissue. In some embodiments, a composition comprising rAAV particles is provided to cells via an in vivo, ex vivo, intraperitoneal, intravenous, intramuscular, intracoronary, subcutaneous, intrathecal, intracranial, intravesicular, or oral delivery method. In some embodiments, a composition comprising rAAV particles is provided to cells by intravenous, intramuscular, intracoronary or intrathecal injection or administration.

In some embodiments, a method of transducing cell with a gene of interest involves providing to the cell any one of the compositions provided herein. In some embodiments, a variant rAAV particle that is used to transduce cells with a gene of interest comprises SEQ ID NO: 11, 12 or 13.

Other aspects of the present disclosure relate to methods and preparations for use with a subject, such as human or non-human subjects, a host cell in situ in a subject, or a host cell derived from a subject. In some embodiments, a subject in which a cell, tissue or organ is transduced is a vertebrate animal (e.g., a mammal or reptile). In some embodiments, a mammalian subject is a human, a non-human primate, a dog, a cat, a hamster, a mouse, a rat, a pig, a horse, a cow, a donkey or a rabbit. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is a model for a particular disease or used to study the pharmacokinetics and/or pharmacokinetics of a protein or siRNA encoded by a gene of interest.

In some embodiments, the subject has or is suspected of having a heart disease that may be treated with gene therapy. In some embodiments, the subject is in any stages of heart failure. In some embodiments, the heart failure is caused by cardiomyopathy. In some embodiments, the heart failure is caused by hypertrophic cardiomyopathy or dilated cardiomyopathy.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host cell, tissue or organ. A therapeutically acceptable amount may be an amount that is capable of treating a disease. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

The following examples are intended to be illustrative of certain embodiments of the present disclosure and are intended to be non-limiting.

### EXAMPLES

### Example 1

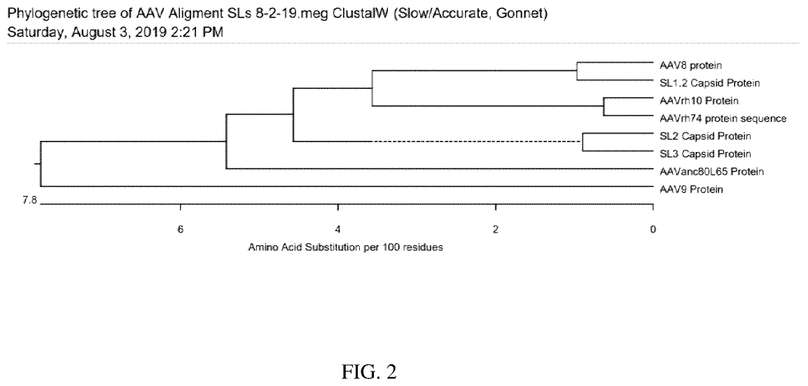

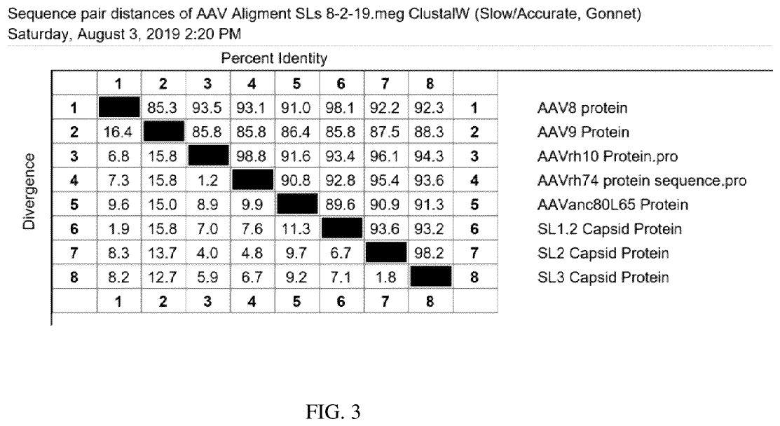

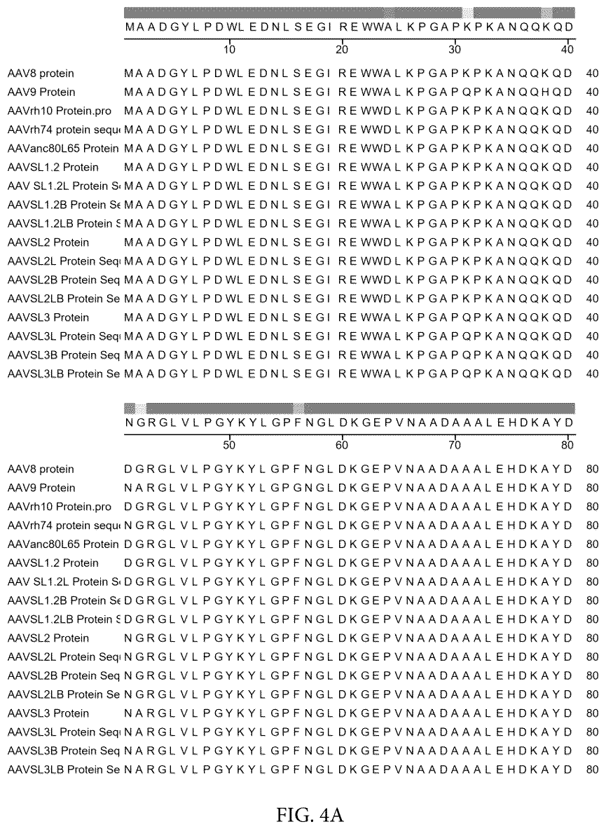

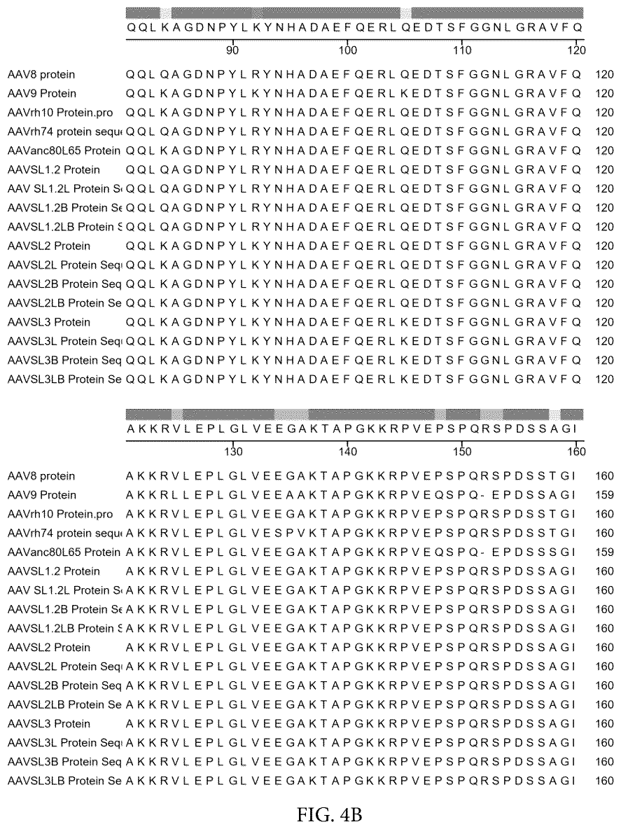

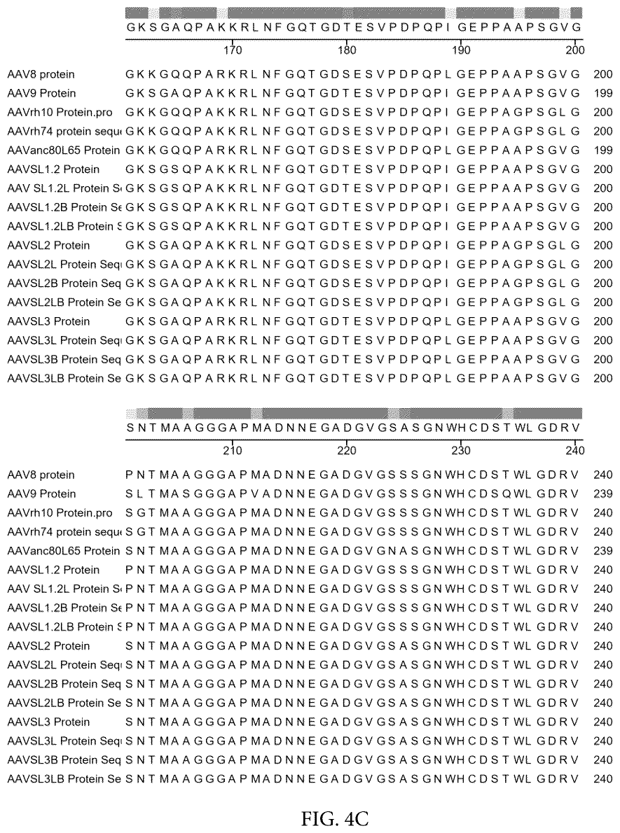

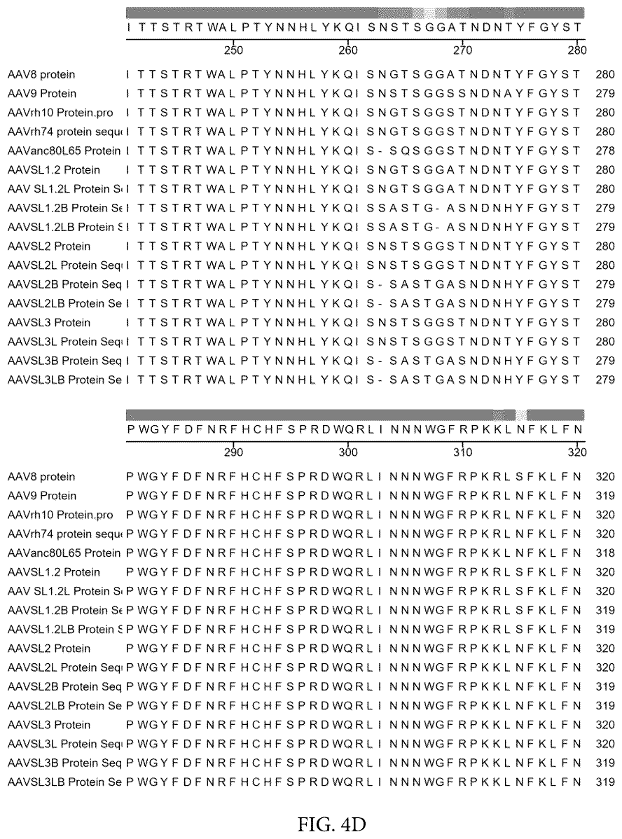

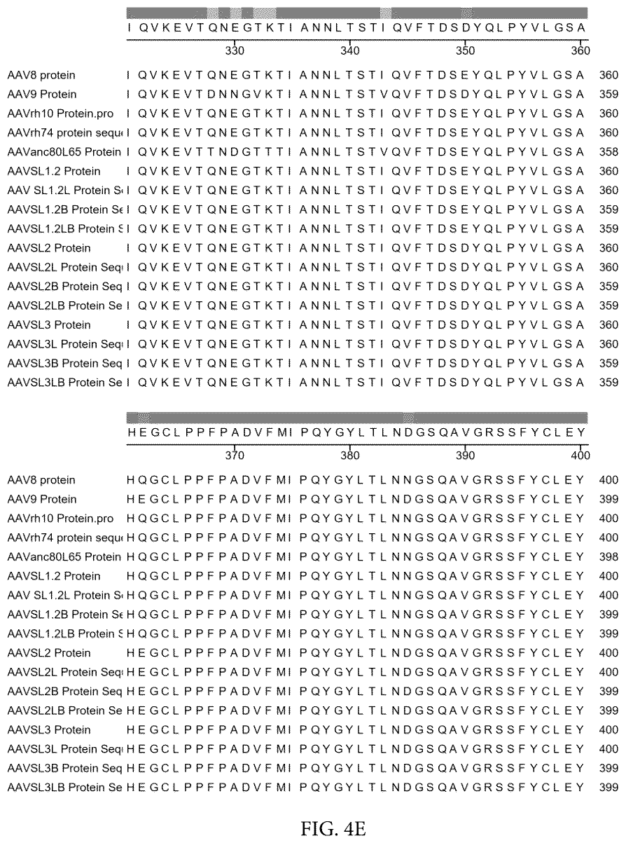

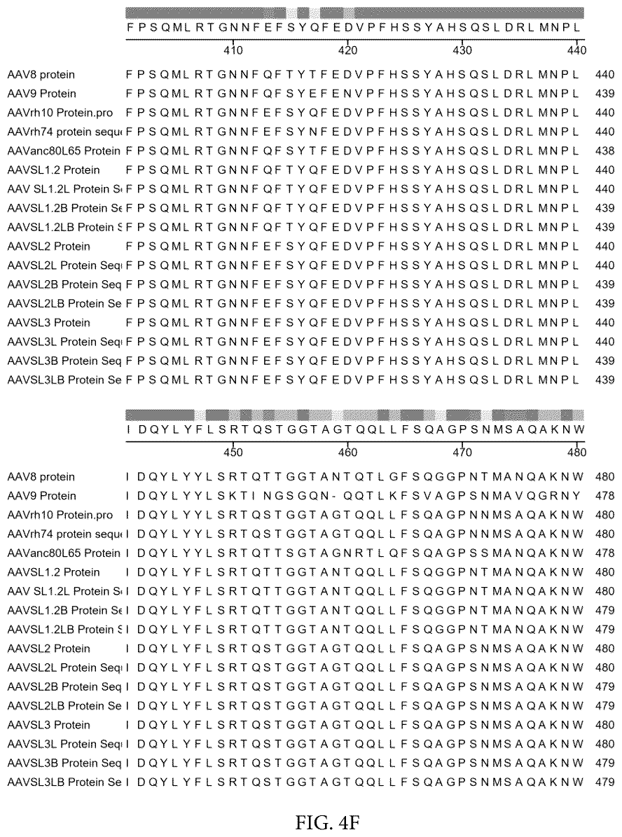

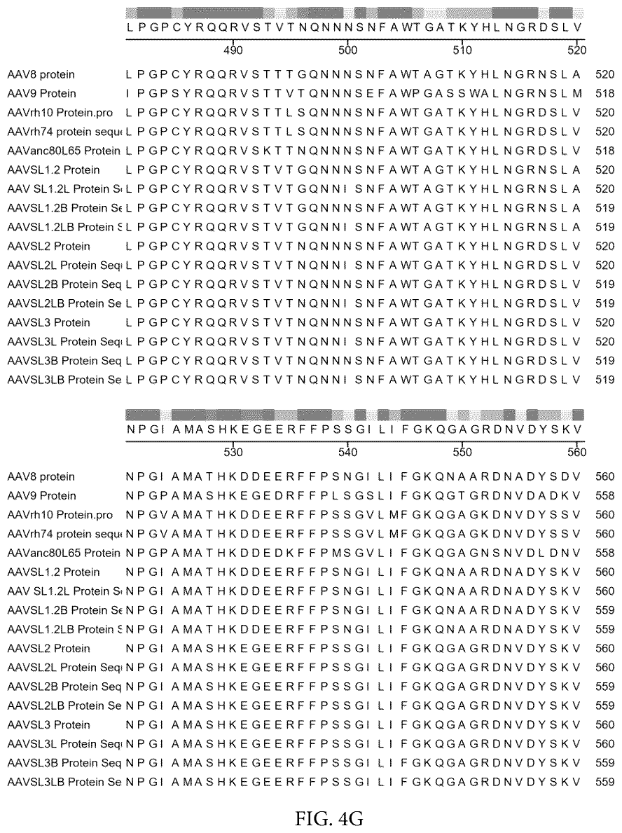

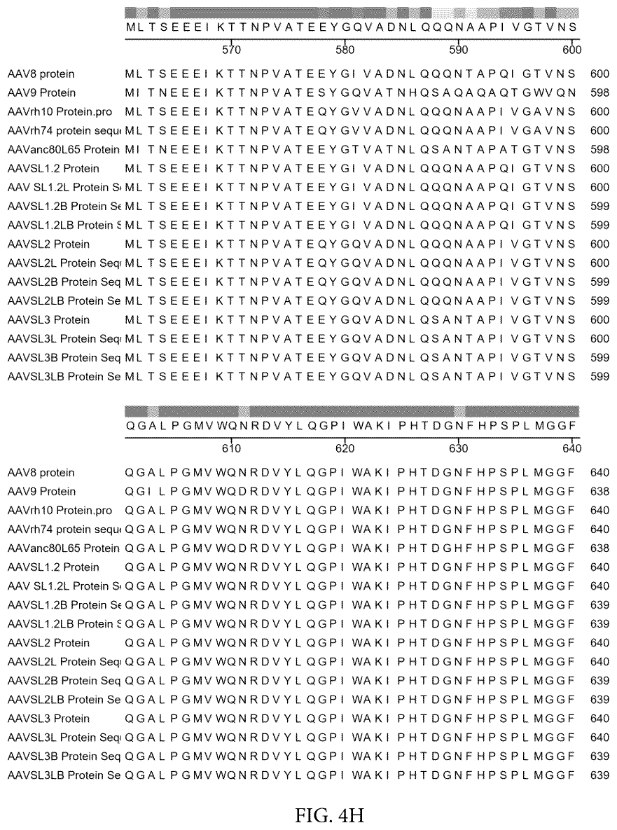

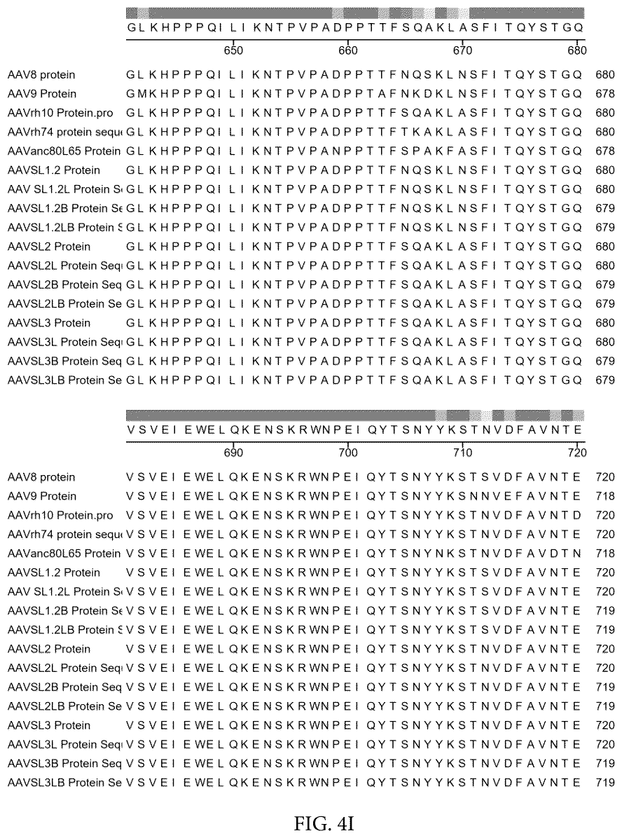

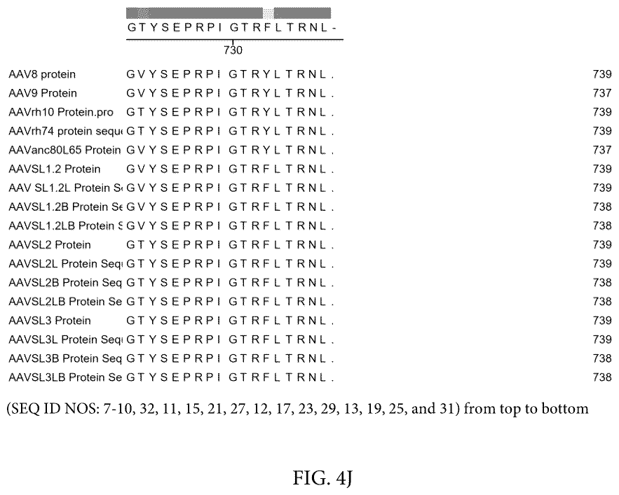

Three AAV variants (“SL1.2” (SEQ ID NO: 11), “SL2” (SEQ ID NO: 12), and “SL3” (SEQ ID NO: 13)) were designed and created to alter the tropisms and/or efficiencies of AAV transduction as compared to AAV8. Transduction efficiency was increased by removing phosphorylation sites that decrease the efficiency of AAV8. To alter tropism, a rational design approach was taken in which the known tropism of AAV8 was compared to the tropism of AAV5, AAV9, AAVrh10, and AAV_ANC80L65, along with the sequence differences of the AAV capsids (see FIG. 1). Differences that correlated with improved cardiac and CNS tropism were then imported into the AAV8 capsid sequence. SL1.2, SL2, and SL3 represent escalating additions of such sequences. AAV-SL1.2 is thus closely related in sequence to AAV8, while AAV-SL2 and AAV-SL3 are more divergent, but more closely related to AAVr10, AAVrh74, and AAV8 than to AAV9 or AAVanc80L65. The phylogeny and percent divergences of SL1.2, SL2, and SL3 relative to the other AAV serotypes are shown in FIGS. 2 and 3, respectively.

### Example 2

To improve the delivery of cardio-protective genes to the heart, mutant capsids were designed to alter regions that appear to be important for cardiac muscle uptake, based on comparisons with naturally occurring AAV serotypes. In particular, the capsid region spanning amino acids Glu578 to Gly596 were varied in the three variants described herein (SL1.2, SL2, and SL3; see Example 1 and FIGS. 1-3).

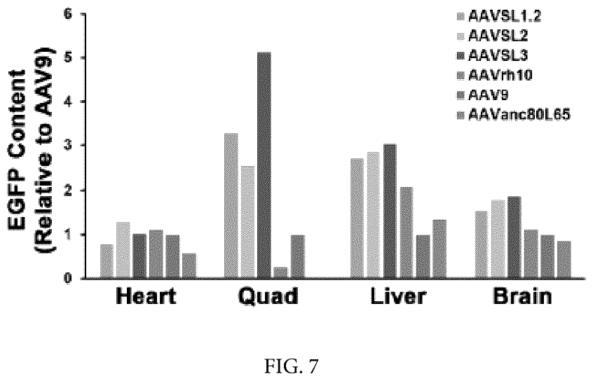

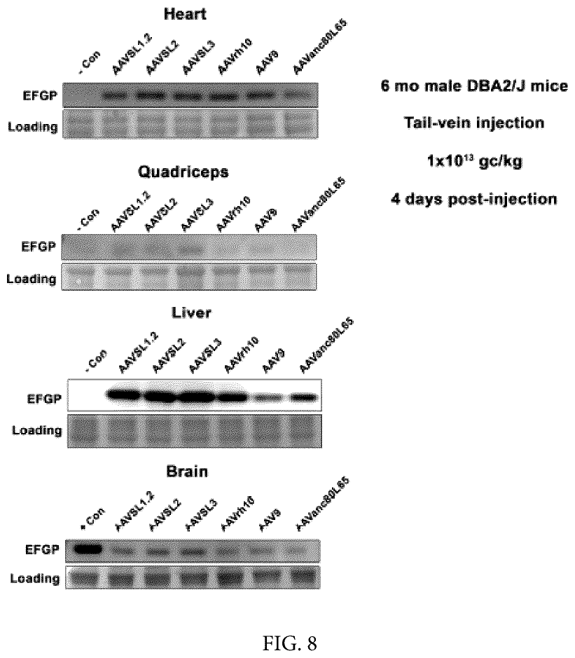

The Western blot quantification (FIG. 7) of the blot shown in FIG. 8 compares the delivery of the three variants (SL1.2, SL2, and SL3) to the uptake of the natural serotypes, AAV9 and AAVrh10, and the engineered variant, AAVanc80L65. AAV9 and AAVrh10 were selected because they are two of the best described AAV serotypes for cardiac delivery. The experiment was conducted using a known sub-saturating dose (1×10¹³ vg/kg) administered by tail vein systemic delivery into 6 month old DBA/2J male mice. Green fluorescent protein (GFP) was used as the reporter protein under control of a beta-actin promoter for expression in all tissues. FIGS. 7 and 8 show that the variant SL2 has the improved uptake in cardiac tissue relative to the control serotypes, while SL3 has uptake more similar to AAV9. It was observed that SL3 has highest uptake in skeletal muscle and brain of any of tested AAV serotype.

As shown in Table 2, it was also observed that the mutations that were introduced into SL2 and SL3 led to a substantial increase in the efficiency of production (e.g., the yield for a given amount of DNA). The efficiency of production for SL2 and SL3 increased such that the yields exceeded that of AAV9 and AAVrh10 by 1.5- to 4-fold, which far exceed the yields of the synthetic variant AAVanc80L65. AAVanc70L65 has been proposed to be more useful for cardiac delivery as compared to AAV8, but, based on the results described herein, is fact be inferior to AAV9, AAVrh10, and the present variants SL2 and SL3. The higher yields of SL2 and SL3 are important for the large-scale production needed for systemic human gene therapy, because these variants will be easier and less expensive to produce.

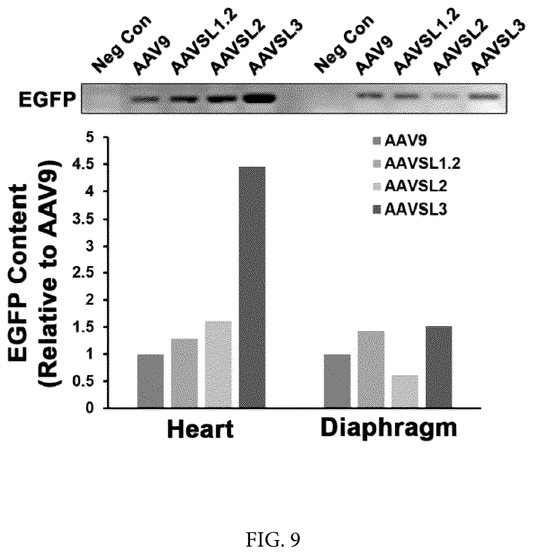

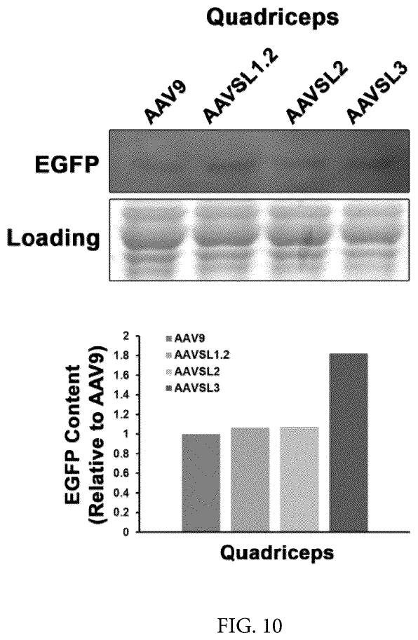

The superiority of SL3 in facilitating uptake in both skeletal and cardiac muscles is shown in FIG. 9 (Western blot and quantification of heart and diaphragm) and FIG. 10 (Western blot and quantification of quadriceps). The data in FIGS. 9 and 10 was obtained by injecting 1×10¹³ vg/kg of AAV9, SL1.2, SL2, or SL3 by tail vein (systemic delivery) into 6 month old DBA/2J male mice. The reporter is GFP under control of the beta-actin promoter for expression in all tissues.

TABLE 2
Yield and efficiency for the variants SL1.2, SL2, and SL3 as
compared to control serotypes AAV9, AAVrh10, and AAVanc80L65.

| | | | Efficiency |
|---|---|---|---|
| Vector Prep | DNA (mg) | yield (gc) | (gc/mg DNA) |
| AAV9 | 1.6 | 1.40E+13 | 8.75E+12 |
| AAVrh10 | 1.6 | 2.67E+13 | 1.67E+13 |
| AAVSL1.2 | 1.6 | 2.17E+13 | 1.36E+13 |
| AAVSL2 | 1.6 | 5.90E+13 | 3.69E+13 |
| AAVSL3 | 1.6 | 4.04E+13 | 2.53E+13 |
| AAVanc80L65 | 1.6 | 3.56E+12 | 2.23E+12 |

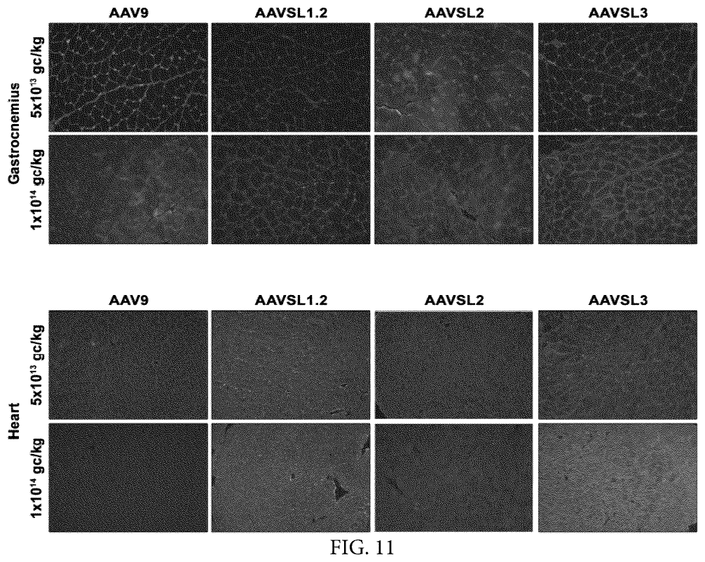

FIG. 11 demonstrates the distribution of Green Fluorescent Protein (GFP; degree of brightness) within skeletal muscles and the heart following systemic injection of AAV9, SL1.2, SL2, or SL3 at 5×10¹³ vg/kg or 1×10¹⁴ vg/kg. The pattern of expression supports the conclusions of the Western blot data, and indicate that SL3 is the superior vector for transduction of the heart and skeletal muscles.

When designing new AAV vectors that are intended to target tissues other than the liver, it is useful to minimize delivery to the liver, which typically takes up more vector than any other tissue. In order to decrease the uptake of SL1.2, SL2, and SL3 by the liver, a single amino acid change (Asparagine 500 to Isoleucine) was introduced to each of SL1.2, SL2, and SL3 (see, e.g., Pulicherla, et al., Mol. Ther. 19:6, 1070-78 (2011)). The resultant vectors are designated herein as “SL1.2L” (SEQ ID NO: 15), “SL2L” (SEQ ID NO: 17), and “SL3L” (SEQ ID NO: 19).

In some applications, specifically where the central nervous system (CNS) is not a target, it is useful to prevent virus crossing the blood-brain barrier and thus minimize delivery to the brain. In order to decrease the uptake of SL1.2, SL2, and SL3 by the brain, seven amino acid changes (Asparagine 263 to Serine; Glycine 264 to Alanine; Threonine 265 to Serine; Serine 266 to Threonine; deletion of Glycine 268; Threonine 270 to Serine; and Threonine 274 to Histidine) were introduced to SL1.2, and eight amino acid changes (Asparagine 263 to Serine; Serine 264 to Alanine; Threonine 265 to Serine; Serine 266 to Threonine; deletion of Glycine 268; Serine 269 to Alanine; Threonine 270 to Serine; and Threonine 274 to Histidine) were introduced into each of SL2 and SL3 (see, e.g., Albright, et al., Mol. Ther. 26:2, 510-23 (2018)). The resultant vectors are designated herein as “SL1.2B” (SEQ ID NO: 21), “SL2B” (SEQ ID NO: 23), and “SL3B” (SEQ ID NO: 25).

Finally, it may in some cases be useful to utilize AAV vectors that have decreased uptake in both the liver and in the brain, relative to naturally occurring AAV serotypes. Accordingly, SL1.2, SL2, and SL3 variants were designed that integrate both the single amino acid change and the eight amino acid changes relating the liver and brain, respectively, described above. The resultant vectors are designated herein as “SL1.2 LB” (SEQ ID NO: 27), “SL2LB” (SEQ ID NO: 29), and “SL3LB” (SEQ ID NO: 31).

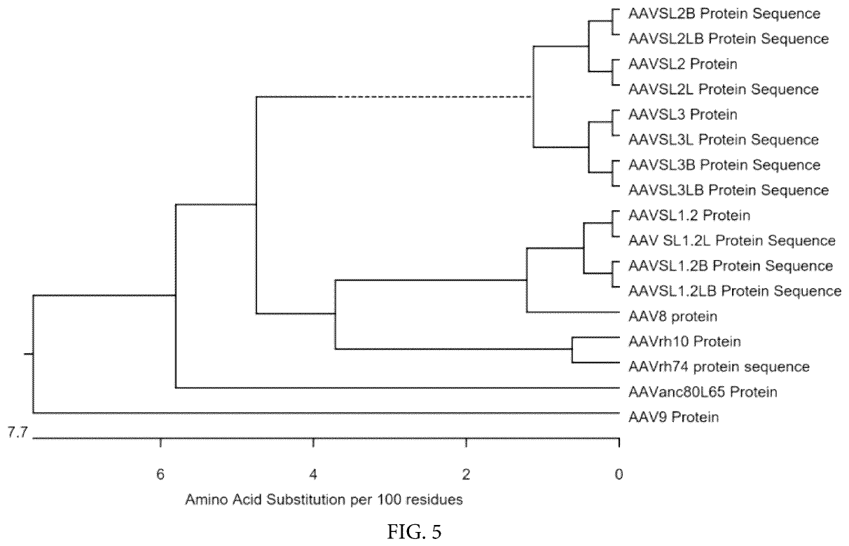

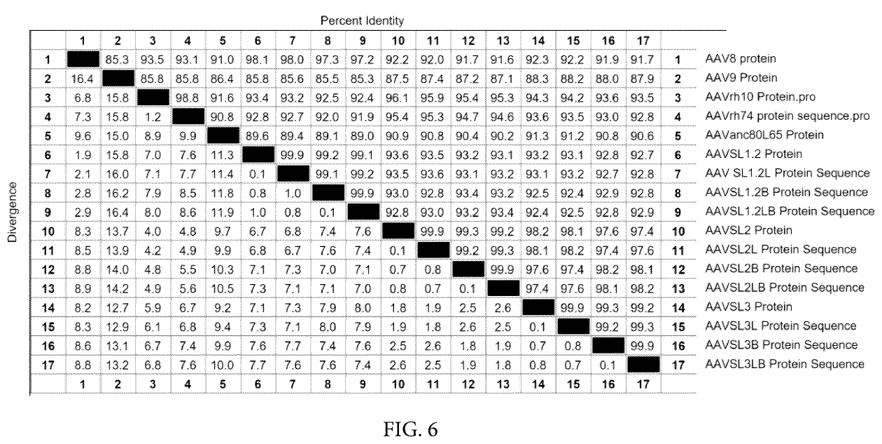

The phylogeny and percent divergences of SL1.2, SL1.2L, SL1.2B, SL1.2 LB, SL2, SL2L, SL2B, SL2LB, SL3, SL3L, SL3B, and SL3LB relative to the other AAV serotypes are shown in FIGS. 5 and 6, respectively.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

### REFERENCES

1. Kay C N, Ryals R C, Aslanidi G V, Min S H, Ruan Q, Sun J, Dyka F M, Kasuga D, Ayala A E, Van Vliet K, Agbandje-McKenna M, Hauswirth W W, Boye S L, Boye S E. Targeting photoreceptors via intravitreal delivery using novel, capsid-mutated AAV vectors. PLoS One. 2013 Apr. 26; 8(4):e62097.

1-28. (canceled)

29. A variant recombinant adeno-associated virus (rAAV) capsid protein comprising the following amino acid substitutions:
K163S, R169K, S180T, L189I, T417Q, Y447F, T462Q, G464L, T494V, D559K, T591A, and Y733F wherein the positions of the amino acid substitutions are based on the wild-type AAV8 capsid protein amino acid sequence set forth in SEQ ID NO: 7.

30. The variant rAAV capsid protein of claim 29, wherein the variant rAAV capsid protein further comprises any one or more of the following amino acid changes: N500I, N263S, G264A, T265S, S266T, deletion of G268, T270S, and T274H, wherein the positions of the changes are based on the wild-type AAV8 capsid protein amino acid sequence set forth in SEQ ID NO: 7.

31. The variant rAAV capsid protein of claim 29, wherein the variant rAAV capsid protein comprises the amino acid sequence set forth in SEQ ID NO: 15.

32. The variant rAAV capsid protein of claim 29, wherein the variant rAAV capsid protein comprises the amino acid sequence set forth in SEQ ID NO: 21.

33. The variant rAAV capsid protein of claim 29, wherein the variant rAAV capsid protein comprises the amino acid sequence set forth in SEQ ID NO: 27.

34. A variant rAAV particle comprising the variant rAAV capsid protein of claim 29.

35. The variant rAAV particle of claim 34, wherein the variant rAAV capsid protein comprises the amino acid sequence set forth in SEQ ID NO: 11.

36. The variant rAAV particle of claim 34, wherein the variant rAAV capsid protein comprises the amino acid sequence set forth in SEQ ID NO: 15.

37. The variant rAAV particle of claim 34, wherein the variant rAAV capsid protein comprises the amino acid sequence set forth in SEQ ID NO: 21 or SEQ ID NO: 27.

38. The variant rAAV particle of claim 34, further comprising a nucleic acid comprising a gene of interest.

39. The variant rAAV particle of claim 38, wherein the nucleic acid is single stranded.

40. The variant rAAV particle of claim 38, wherein the nucleic acid is double stranded.

41. A composition comprising a plurality of the variant rAAV particles of claim 34.

42. The composition of claim 41, further comprising a pharmaceutically acceptable carrier.

43. A method of transducing a cell with a gene of interest, the method comprising providing to the cell the composition of claim 41, wherein the rAAV particles in the composition comprise a nucleic acid comprising a gene of interest.

44. The method of claim 43, wherein the cell is a mammalian cell.

45. The method of claim 44, wherein the cell is a human cell.

46. The method of claim 43, wherein the gene of interest encodes a therapeutic protein.

47. The method of claim 46, wherein the therapeutic protein is an antibody or antibody fragment, a peptibody, a growth factor, a hormone, a membrane protein, a cytokine, a chemokine, an activating or inhibitory peptide acting on cell surface receptors or ion channels, a cell-permeant peptide targeting intracellular processes, an enzyme, or a nuclease or other protein used for gene editing.

48. The method of claim 43, wherein the gene of interest is a cardioprotective gene.


SEQUENCE LISTING (32 sequences)

SEQ ID NO: 1 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggctcacagcccgccaaaaaaagactcaattttggtcagactggcgacacagagtcagttccagaccctcaacctatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttaccagttcgaggacgtgcctttccacagcagctacgcccacagccagagcttggaccggctgatgaatcctctgattgaccagtacctgtacttcttgtctcggactcaaacaacaggaggcacggcaaatacgcagcagctgctattcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcaaagtcatgctcaccagcgaggaagaaatcaaaaccactaaccctgtggctacagaggaatacggtatcgtggcagataacttgcagcagcaaaacgccgctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 2 - protein, 738 aa, synthetic construct
  REGION 1..738: note = Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSAGIGKSGSQPAKKRLNFGQTGDTESVPDPQPIGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYQFEDVPFHSSYAHSQSLDRLMNPLIDQYLYFLSRTQTTGGTANTQQLLFSQGGPNTMANQAKNWLPGPCYRQQRVSTVTGQNNNSNFAWTAGTKYHLNGRNSLANPGIAMATHKDDEERFFPSNGILIFGKQNAARDNADYSKVMLTSEEEIKTTNPVATEEYGIVADNLQQQNAAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 3 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggacttgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgaaaaagagactcaactttgggcagactggcgactcagagtcagtgcccgaccctcaaccaatcggagaaccccccgcaggcccctctggtctgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctccaacagcacttcgggaggaagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacaacagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaacagtacggccaagtggccgataacctgcaacagcaaaacgccgctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 4 - protein, 738 aa, synthetic construct
  REGION 1..738: note = Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLKYNHADAEFQERLQEDTSFGGNLGRAVFQAKKRVLEPLGLVEEGAKTAPGKKRPVEPSPQRSPDSSAGIGKSGAQPAKKRLNFGQTGDSESVPDPQPIGEPPAGPSGLGSNTMAAGGGAPMADNNEGADGVGSASGNWHCDSTWLGDRVITTSTRTWALPTYNNHLYKQISNSTSGGSTNDNTYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNNSNFAWTGATKYHLNGRDSLVNPGIAMASHKEGEERFFPSSGILIFGKQGAGRDNVDYSKVMLTSEEEIKTTNPVATEQYGQVADNLQQQNAAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 5 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggctttgaaacctggagccccgcaacccaaagccaaccagcaaaagcaggacaacgctcggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgaaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgagaaagagactcaactttgggcagactggcgacacagagtcagtgcccgaccctcaaccactcggagaaccccccgcagccccctctggtgtgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctccaacagcacttcgggaggaagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacaacagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaagaatacggccaagtggccgataacctgcaaagtgccaacacggctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 6 - protein, 738 aa, synthetic construct
  REGION 1..738: note = Synthetic
MAADGYLPDWLEDNLSEGIREWWALKPGAPQPKANQQKQDNARGLVLPGYKYLGPFNGLDKGEPVNAADAAALEHDKAYDQQLKAGDNPYLKYNHADAEFQERLKEDTSFGGNLGRAVFQAKKRVLEPLGLVEEGAKTAPGKKRPVEPSPQRSPDSSAGIGKSGAQPARKRLNFGQTGDTESVPDPQPLGEPPAAPSGVGSNTMAAGGGAPMADNNEGADGVGSASGNWHCDSTWLGDRVITTSTRTWALPTYNNHLYKQISNSTSGGSTNDNTYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNNSNFAWTGATKYHLNGRDSLVNPGIAMASHKEGEERFFPSSGILIFGKQGAGRDNVDYSKVMLTSEEEIKTTNPVATEEYGQVADNLQSANTAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 7 - protein, 738 aa, synthetic construct
  REGION 1..738: note = Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - protein, 736 aa, synthetic construct
  REGION 1..736: note = Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 738 aa, synthetic construct
  REGION 1..738: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 10 - protein, 738 aa, synthetic construct
  REGION 1..738: note = Synthetic
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 11 - protein, 738 aa, synthetic construct; identical to SEQ ID NO: 2
  REGION 1..738: note = Synthetic

SEQ ID NO: 12 - protein, 738 aa, synthetic construct; identical to SEQ ID NO: 4
  REGION 1..738: note = Synthetic

SEQ ID NO: 13 - protein, 738 aa, synthetic construct; identical to SEQ ID NO: 6
  REGION 1..738: note = Synthetic

SEQ ID NO: 14 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggctcacagcccgccaaaaaaagactcaattttggtcagactggcgacacagagtcagttccagaccctcaacctatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttaccagttcgaggacgtgcctttccacagcagctacgcccacagccagagcttggaccggctgatgaatcctctgattgaccagtacctgtacttcttgtctcggactcaaacaacaggaggcacggcaaatacgcagcagctgctattcagccaaggtgggcctaatacaatggccaatcaggcaaagaactggctgccaggaccctgttaccgccaacaacgcgtctcaacggtaaccgggcaaaacaacatcagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcaaagtcatgctcaccagcgaggaagaaatcaaaaccactaaccctgtggctacagaggaatacggtatcgtggcagataacttgcagcagcaaaacgccgctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 15 - protein, 738 aa, synthetic construct
  REGION 1..738: note = Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSAGIGKSGSQPAKKRLNFGQTGDTESVPDPQPIGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYQFEDVPFHSSYAHSQSLDRLMNPLIDQYLYFLSRTQTTGGTANTQQLLFSQGGPNTMANQAKNWLPGPCYRQQRVSTVTGQNNISNFAWTAGTKYHLNGRNSLANPGIAMATHKDDEERFFPSNGILIFGKQNAARDNADYSKVMLTSEEEIKTTNPVATEEYGIVADNLQQQNAAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 16 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggacttgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgaaaaagagactcaactttgggcagactggcgactcagagtcagtgcccgaccctcaaccaatcggagaaccccccgcaggcccctctggtctgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctccaacagcacttcgggaggaagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacatcagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaacagtacggccaagtggccgataacctgcaacagcaaaacgccgctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 17 - protein, 738 aa, synthetic construct
  REGION 1..738: note = Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLKYNHADAEFQERLQEDTSFGGNLGRAVFQAKKRVLEPLGLVEEGAKTAPGKKRPVEPSPQRSPDSSAGIGKSGAQPAKKRLNFGQTGDSESVPDPQPIGEPPAGPSGLGSNTMAAGGGAPMADNNEGADGVGSASGNWHCDSTWLGDRVITTSTRTWALPTYNNHLYKQISNSTSGGSTNDNTYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNISNFAWTGATKYHLNGRDSLVNPGIAMASHKEGEERFFPSSGILIFGKQGAGRDNVDYSKVMLTSEEEIKTTNPVATEQYGQVADNLQQQNAAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 18 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggctttgaaacctggagccccgcaacccaaagccaaccagcaaaagcaggacaacgctcggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgaaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgagaaagagactcaactttgggcagactggcgacacagagtcagtgcccgaccctcaaccactcggagaaccccccgcagccccctctggtgtgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctccaacagcacttcgggaggaagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacatcagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaagaatacggccaagtggccgataacctgcaaagtgccaacacggctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 19 - protein, 738 aa, synthetic construct
  REGION 1..738: note = Synthetic
MAADGYLPDWLEDNLSEGIREWWALKPGAPQPKANQQKQDNARGLVLPGYKYLGPFNGLDKGEPVNAADAAALEHDKAYDQQLKAGDNPYLKYNHADAEFQERLKEDTSFGGNLGRAVFQAKKRVLEPLGLVEEGAKTAPGKKRPVEPSPQRSPDSSAGIGKSGAQPARKRLNFGQTGDTESVPDPQPLGEPPAAPSGVGSNTMAAGGGAPMADNNEGADGVGSASGNWHCDSTWLGDRVITTSTRTWALPTYNNHLYKQISNSTSGGSTNDNTYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNISNFAWTGATKYHLNGRDSLVNPGIAMASHKEGEERFFPSSGILIFGKQGAGRDNVDYSKVMLTSEEEIKTTNPVATEEYGQVADNLQSANTAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 20 - DNA, 2214 bp, synthetic construct
  misc_feature 1..2214: note = Synthetic
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggctcacagcccgccaaaaaaagactcaattttggtcagactggcgacacagagtcagttccagaccctcaacctatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctcctcagcatccacaggagccagtaacgacaaccattacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttaccagttcgaggacgtgcctttccacagcagctacgcccacagccagagcttggaccggctgatgaatcctctgattgaccagtacctgtacttcttgtctcggactcaaacaacaggaggcacggcaaatacgcagcagctgctattcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcaaagtcatgctcaccagcgaggaagaaatcaaaaccactaaccctgtggctacagaggaatacggtatcgtggcagataacttgcagcagcaaaacgccgctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 21 - protein, 737 aa, synthetic construct
  REGION 1..737: note = Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSAGIGKSGSQPAKKRLNFGQTGDTESVPDPQPIGEPPAAPSGVGPNTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYQFEDVPFHSSYAHSQSLDRLMNPLIDQYLYFLSRTQTTGGTANTQQLLFSQGGPNTMANQAKNWLPGPCYRQQRVSTVTGQNNNSNFAWTAGTKYHLNGRNSLANPGIAMATHKDDEERFFPSNGILIFGKQNAARDNADYSKVMLTSEEEIKTTNPVATEEYGIVADNLQQQNAAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 22 - DNA, 2214 bp, synthetic construct
  misc_feature 1..2214: note = Synthetic
  source 1..2214: mol_type = other DNA
atggctgccgatggttatcttccagattggctcgaggacaacctctctgagggcattcgcgagtggtgggacttgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgaaaaagagactcaactttgggcagactggcgactcagagtcagtgcccgaccctcaaccaatcggagaaccccccgcaggcccctctggtctgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctcctcagcatccacaggagccagtaacgacaaccattacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacaatagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaacagtacggccaagtggccgataacctgcaacagcaaaacgccgctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 23 - protein, 737 aa, synthetic construct
  REGION 1..737: note = Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLKYNHADAEFQERLQEDTSFGGNLGRAVFQAKKRVLEPLGLVEEGAKTAPGKKRPVEPSPQRSPDSSAGIGKSGAQPAKKRLNFGQTGDSESVPDPQPIGEPPAGPSGLGSNTMAAGGGAPMADNNEGADGVGSASGNWHCDSTWLGDRVITTSTRTWALPTYNNHLYKQISSASTGASNDNHYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNNSNFAWTGATKYHLNGRDSLVNPGIAMASHKEGEERFFPSSGILIFGKQGAGRDNVDYSKVMLTSEEEIKTTNPVATEQYGQVADNLQQQNAAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 24 - DNA, 2214 bp, synthetic construct
  misc_feature 1..2214: note = Synthetic
  source 1..2214: mol_type = other DNA
atggctgccgatggttatcttccagattggctcgaggacaacctctctgagggcattcgcgagtggtgggctttgaaacctggagccccgcaacccaaagccaaccagcaaaagcaggacaacgctcggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgaaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgagaaagagactcaactttgggcagactggcgacacagagtcagtgcccgaccctcaaccactcggagaaccccccgcagccccctctggtgtgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctcctcagcatccacaggagccagtaacgacaaccattacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacaatagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaagaatacggccaagtggccgataacctgcaaagtgccaacacggctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 25 - protein, 737 aa, synthetic construct
  REGION 1..737: note = Synthetic
MAADGYLPDWLEDNLSEGIREWWALKPGAPQPKANQQKQDNARGLVLPGYKYLGPFNGLDKGEPVNAADAAALEHDKAYDQQLKAGDNPYLKYNHADAEFQERLKEDTSFGGNLGRAVFQAKKRVLEPLGLVEEGAKTAPGKKRPVEPSPQRSPDSSAGIGKSGAQPARKRLNFGQTGDTESVPDPQPLGEPPAAPSGVGSNTMAAGGGAPMADNNEGADGVGSASGNWHCDSTWLGDRVITTSTRTWALPTYNNHLYKQISSASTGASNDNHYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNNSNFAWTGATKYHLNGRDSLVNPGIAMASHKEGEERFFPSSGILIFGKQGAGRDNVDYSKVMLTSEEEIKTTNPVATEEYGQVADNLQSANTAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 26 - DNA, 2214 bp, synthetic construct
  misc_feature 1..2214: note = Synthetic
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggctcacagcccgccaaaaaaagactcaattttggtcagactggcgacacagagtcagttccagaccctcaacctatcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctcctcagcatccacaggagccagtaacgacaaccattacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttaccagttcgaggacgtgcctttccacagcagctacgcccacagccagagcttggaccggctgatgaatcctctgattgaccagtacctgtacttcttgtctcggactcaaacaacaggaggcacggcaaatacgcagcagctgctattcagccaaggtgggcctaatacaatggccaatcaggcaaagaactggctgccaggaccctgttaccgccaacaacgcgtctcaacggtaaccgggcaaaacaacatcagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcaaagtcatgctcaccagcgaggaagaaatcaaaaccactaaccctgtggctacagaggaatacggtatcgtggcagataacttgcagcagcaaaacgccgctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 27 - protein, 737 aa, synthetic construct
  REGION 1..737: note = Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSAGIGKSGSQPAKKRLNFGQTGDTESVPDPQPIGEPPAAPSGVGPNTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYQFEDVPFHSSYAHSQSLDRLMNPLIDQYLYFLSRTQTTGGTANTQQLLFSQGGPNTMANQAKNWLPGPCYRQQRVSTVTGQNNISNFAWTAGTKYHLNGRNSLANPGIAMATHKDDEERFFPSNGILIFGKQNAARDNADYSKVMLTSEEEIKTTNPVATEEYGIVADNLQQQNAAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 28 - DNA, 2214 bp, synthetic construct
  misc_feature 1..2214: note = Synthetic
  source 1..2214: mol_type = other DNA
atggctgccgatggttatcttccagattggctcgaggacaacctctctgagggcattcgcgagtggtgggacttgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgaaaaagagactcaactttgggcagactggcgactcagagtcagtgcccgaccctcaaccaatcggagaaccccccgcaggcccctctggtctgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctcctcagcatccacaggagccagtaacgacaaccattacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacatcagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaacagtacggccaagtggccgataacctgcaacagcaaaacgccgctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 29 - protein, 737 aa, synthetic construct
  REGION 1..737: note = Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLKYNHADAEFQERLQEDTSFGGNLGRAVFQAKKRVLEPLGLVEEGAKTAPGKKRPVEPSPQRSPDSSAGIGKSGAQPAKKRLNFGQTGDSESVPDPQPIGEPPAGPSGLGSNTMAAGGGAPMADNNEGADGVGSASGNWHCDSTWLGDRVITTSTRTWALPTYNNHLYKQISSASTGASNDNHYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNISNFAWTGATKYHLNGRDSLVNPGIAMASHKEGEERFFPSSGILIFGKQGAGRDNVDYSKVMLTSEEEIKTTNPVATEQYGQVADNLQQQNAAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 30 - DNA, 2214 bp, synthetic construct
  misc_feature 1..2214: note = Synthetic
  source 1..2214: mol_type = other DNA
atggctgccgatggttatcttccagattggctcgaggacaacctctctgagggcattcgcgagtggtgggctttgaaacctggagccccgcaacccaaagccaaccagcaaaagcaggacaacgctcggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgaagtataaccacgccgacgccgagtttcaggagcgtctgaaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctgcgggcatcggcaagtcgggcgcacagcccgcgagaaagagactcaactttgggcagactggcgacacagagtcagtgcccgaccctcaaccactcggagaaccccccgcagccccctctggtgtgggatctaatacaatggctgcaggcggtggcgctccaatggcagacaataacgaaggcgccgacggagtgggtagtgcctcaggaaattggcattgcgattccacatggctgggcgacagagtcatcaccaccagcacccgaacctgggccctccccacctacaacaaccacctctacaagcaaatctcctcagcatccacaggagccagtaacgacaaccattacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgcagaatgaaggcaccaagaccatcgccaataaccttaccagcacgattcaggtctttacggactcggactaccagctcccgtacgtcctcggctctgcgcacgagggctgcctgcctccgttcccggcggacgtcttcatgattcctcagtacgggtacctgactctgaacgatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtacttcctgtctcggactcagtccacgggaggtaccgcaggaactcagcagttgctattttctcaggccgggcctagcaacatgtcggctcaggccaaaaactggctacccgggccctgctaccggcagcaacgcgtctccacggtaaccaaccaaaataacatcagcaactttgcctggaccggtgccaccaagtatcatctgaatggcagagactctctggtaaatcccggtatcgctatggcaagccacaaggaaggagaagagcgattttttccgtccagcggaatcttaatttttgggaaacagggagctggaagagacaacgtggactatagcaaagttatgctaaccagtgaggaagaaattaaaaccaccaacccagtggccacagaagaatacggccaagtggccgataacctgcaaagtgccaacacggctcctattgtagggactgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagaaggcacttattctgagcctcgccccatcggcacccgtttcctcacccgtaatctgtaa

SEQ ID NO: 31 - protein, 737 aa, synthetic construct
  REGION 1..737: note = Synthetic
MAADGYLPDWLEDNLSEGIREWWALKPGAPQPKANQQKQDNARGLVLPGYKYLGPFNGLDKGEPVNAADAAALEHDKAYDQQLKAGDNPYLKYNHADAEFQERLKEDTSFGGNLGRAVFQAKKRVLEPLGLVEEGAKTAPGKKRPVEPSPQRSPDSSAGIGKSGAQPARKRLNFGQTGDTESVPDPQPLGEPPAAPSGVGSNTMAAGGGAPMADNNEGADGVGSASGNWHCDSTWLGDRVITTSTRTWALPTYNNHLYKQISSASTGASNDNHYFGYSTPWGYFDFNRFHCHFSPRDWQRLINNNWGFRPKKLNFKLFNIQVKEVTQNEGTKTIANNLTSTIQVFTDSDYQLPYVLGSAHEGCLPPFPADVFMIPQYGYLTLNDGSQAVGRSSFYCLEYFPSQMLRTGNNFEFSYQFEDVPFHSSYAHSQSLDRLMNPLIDQYLYFLSRTQSTGGTAGTQQLLFSQAGPSNMSAQAKNWLPGPCYRQQRVSTVTNQNNISNFAWTGATKYHLNGRDSLVNPGIAMASHKEGEERFFPSSGILIFGKQGAGRDNVDYSKVMLTSEEEIKTTNPVATEEYGQVADNLQSANTAPIVGTVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRFLTRNL

SEQ ID NO: 32 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL